(19) United States
(12) Patent Application Publication
(10) Pub. No.: US 2023/0277670 A1
(43) Pub. Date: Sep. 7, 2023

(54) CHIMERIC MOLECULES PROVIDING TARGETED COSTIMULATION FOR ADOPTIVE CELL THERAPY

(71) Applicants: John BRIDGEMAN, Manchester (GB); Robert HAWKINS, Manchester (GB); Ruben RODRIGUEZ, Dallas, TX (US); Instil Bio (UK) Limited, Manchester (GB)

(72) Inventors: John Bridgeman, Manchester (GB); Robert Hawkins, Manchester (GB); Ruben Rodriguez, Dallas, TX (US)

(21) Appl. No.: 18/005,330

(22) PCT Filed: Jul. 16, 2021
(86) PCT No.: PCT/US2021/042079

§ 371 Date: Jan. 12, 2023

Related U.S. Application Data
(60) Provisional application No. 63/053,498, filed on Jul. 17, 2020.

(51) Int. Cl. A61K 39/00; C07K 14/715; C07K 14/705; C07K 14/725; C12N 5/0783

(52) U.S. Cl. CPC A61K 39/4632; C07K 14/7151; C07K 14/70521; C07K 14/7051; C12N 5/0636

(57) ABSTRACT

The present invention relates to a chimeric molecule useful in adoptive cell therapy (ACT), and cells comprising the same. The chimeric molecule can act as a modulator of cellular activity enhancing responses when an endogenous T-cell receptor (TCR) is engaged with its cognate antigen. The present invention also provides proteins, nucleic acids encoding the chimeric molecule and therapeutic uses thereof.

### RELATED APPLICATIONS AND INCORPORATION BY REFERENCE

This application claims the benefit of priority from U.S. Patent Application Ser. No. 63/053,498 filed Jul. 17, 2020, the contents of which are incorporated herein by reference in their entireties.

Reference is made to GB patent application Serial No. 1900858.0, filed 22 Jan. 2019, U.S. patent application Ser. No. 62/951,770, filed 20 Dec. 2019, International application PCT/GB2020/050120, filed 20 Jan. 2020, and U.S. provisional patent application 63/053,494, filed Jul. 17, 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a chimeric molecule useful in adoptive cell therapy (ACT), and cells comprising the same. The chimeric molecule can act as a modulator of cellular activity enhancing responses when an endogenous T-cell receptor (TCR) is engaged with its cognate antigen. The present invention also provides proteins, nucleic acids encoding the chimeric molecule and therapeutic uses thereof.

### BACKGROUND OF THE INVENTION

Adoptive cell therapy (ACT) using autologous T-cells to mediate cancer regression has shown much promise in early clinical trials. Several general approaches have been taken such as the use of naturally occurring tumor reactive or tumor infiltrating lymphocytes (TILs) expanded ex vivo. Additionally, T-cells may be genetically modified to retarget them towards defined tumor antigens. This can be done via the gene transfer of peptide (p)-major histocompatibility complex (MHC) specific T-cell Receptors (TCRs) or synthetic fusions between tumor specific single chain antibody fragment (scFv) and T-cell signaling domains (e.g. CD3ζ), the latter being termed chimeric antigen receptors (CARs).

TIL and TCR transfer has proven particularly good when targeting melanoma (Rosenberg et al. 2011; Morgan 2006), whereas CAR therapy has shown much promise in the treatment of certain B-cell malignancies (Grupp et al. 2013).

Costimulatory signals are useful to achieve robust CAR T cell expansion, function, persistence and antitumor activity. The success of CAR therapy in leukemia has been partly attributed to the incorporation of costimulatory domains (e.g. CD28 or CD137) into the CAR construct, signals from which synergize with the signal provided by CD3ζ to enhance anti-tumor activity. The basis of this observation relates to the classical signal 1/signal 2 paradigm of T-cell activation. Here signal 1, provided by the TCR complex, synergizes with signal 2 provided by costimulatory receptors such as CD28, CD137 or CD134 to permit the cells to undergo clonal expansion, IL2 production and long term survival without the activation induced cell death (AICD) associated with signal 1 alone. Furthermore the involvement of signal 2 enhances the signal generated through signal 1 allowing the cells to respond better to low avidity interactions such as those encountered during anti-tumor responses.

Targeted costimulation will have beneficial effects for non-CAR-based T-cell therapies. For example, incorporating costimulatory domains into a chimeric TCR has been shown to enhance responses of T-cells towards pMHC (Govers 2014). While tumor infiltrating lymphocytes (TILs) utilize their endogenous TCRs to mediate tumor recognition, it has not been possible to engineer the endogenous TCR. Thus TIL are subject to substantial limitations as tumor cells express very few costimulatory ligands. The ability to induce targeted costimulation of TIL, or indeed any other adoptive T-cell therapy product, would be beneficial.

Citation or identification of any document in this application is not an admission that such document is available as prior art to the present invention.

### SUMMARY OF THE INVENTION

The present invention relates to chimeric molecules, in particular chimeric proteins, designed to provide costimulation when the endogenous TCR is engaged with its cognate antigen. Mechanistically, the proposed constructs may be incorporated in the endogenous TCR complex. When the endogenous TCR complex machinery is engaged with their cognate antigen, the TCR receptor complex aggregates, forcing the clustering of these chimeric constructs. This clustering results in the activation of their signaling domains, causing an increase in costimulation. This costimulation manifests itself in a measurable improvement in the effector function of the recipient T cell: increased in activation markers, increase cytokine secretion (IL-2 in particular) and increased proliferation.

The present invention relates to a chimeric molecule, advantageously a chimeric protein, that provides costimulation to the T cell when the endogenous T cell receptor is engaged. This molecule may comprise a TCR clustering domain and a signaling domain that may contain a CD40 intracellular domain or signaling fragment thereof.

The TCR clustering domain may be one or more of the proteins typically found in the TCR complex, such as but not limited to, CD3D, CD3E, CD3G, CD3Z, CD3-eta and the constant chains of pre-TCR alpha (PTCRA) TCR alpha, TCR beta, TCR gamma or TCR delta.

The signaling domain may also comprise, an additional full length costimulatory domain, including but not limited to CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6.

While CD3D, CD3E, CD3G, CD3Z work alone; the constructs containing TCR constant chains (either alpha/beta or gamma/delta) are preferably co-expressed with their respective partner in bicistronic configuration: TCR alpha with TCR beta and TCR gamma with TCR delta. Therefore, TCR alpha containing constructs are advantageously co-expressed with TCR beta and vice versa; and TCR gamma containing constructs should be co-expressed with TCR delta and vice versa. In the context of TILs and any other alpha-beta T cells; the preferred configuration includes TCR gamma-delta; and in gamma-delta T cells, the preferred configuration includes TCR alpha-beta to minimize interference/disruption with the endogenous TCR machinery and the TCR pairing.

For CD3D, CD3E, CD3G and CD3Z, the transmembrane and extracellular portions are advantageously utilized. However, the present invention also contemplates portions or the totality of their intracellular components, which could potentially minimize the disruption of the endogenous TCR complex signaling or help to further amplify the endogenous TCR signaling.

In another aspect, the invention provides a chimeric protein comprising a clustering domain and a signaling domain that may contain a CD40 intracellular domain or signaling fragment thereof. In certain embodiments, the clustering domain is capable of oligomerization and/or self assembly. In certain embodiments, clustering comprises formation of a homodimer or homotrimer. In certain embodiments, clustering comprises oligomerization with a different protein to form a heterodimer or heterotrimer. In certain embodiments, the chimeric protein is constitutive as signaling, for example independent of receptor engagement by an extracellular ligand or independent of receptor engagement by an extracellular ligand attached to a different cell. In certain embodiments, the clustering domain comprises a transmembrane domain. In certain embodiments, the clustering domain comprises a transmembrane domain and further comprises activating mutations that promote dimerization or oligomerization. In certain embodiments, the clustering domain comprises an extracellular domain, such as but not limited to an extracellular domain of a receptor. In certain embodiments, the clustering domain comprises an extracellular domain of a receptor and further comprises activating mutations in the extracellular domain that promote dimerization or oligomerization. In certain embodiments, the clustering domain comprises a leucine zipper. In certain embodiments, the leucine zipper comprises or constitutes a transmembrane domain. In certain embodiments, the leucine zipper comprises or constitutes a soluble domain. Non-limiting examples of clustering domains include clustering domains of the thrombopoietin receptor (TpoR), erythropoietin receptor (EpoR), growth hormone receptor (GHR), glycophorin A (GPA) transmembrane domain, and activating mutants thereof. In certain embodiments, clustering may be modulated by a small molecule. In certain embodiments, clustering may be modulated by post-translational modifications.

In another aspect, the invention provides a chimeric protein which comprises an extracellular ligand binding domain linked to an intracellular signaling domain by a transmembrane domain. In certain embodiments, the extracellular ligand binding domain is selected or engineered to bind to an extracellular ligand that maintains two or more copies of the chimeric protein in proximity to one another such that the signaling domain is activated. The extracellular ligand binding domain is considered one part of a specific binding pair (sbp) and the extracellular ligand is the second part of the specific binding pair. In certain embodiments, one member of the sbp comprises a protein or receptor or extracellular portion thereof and the second sbp comprises a binding protein specific for the first member of the sbp. In certain embodiments, the extracellular sbp is bivalent. In certain embodiments, the extracellular sbp is trivalent. Nonlimiting examples of extracellular ligands include antibodies and bivalent antigen binding fragments thereof. Non-limiting examples of extracellular ligand binding domains of chimeric proteins of the invention (i.e., sbp members) include, without limitation, NKG2A, CD27, CD137, GITR, PD-1, PD-L1, FasL, OX40, CTLA4, ICOS, CD40, EGFR, HER2 and extracellular portions thereof. Complementary sbp members include, without limitation, pembrolizumab for PD1, trastuzumab for HER2, cetuximab for EGFR, tremelimumab for CTLA4, varlilumab for CD27, and urelumab for CD137. In certain embodiments, the intracellular signaling domain comprises a CD40 intracellular domain or signaling fragment thereof.

In certain embodiments of the invention, the CD40 signaling domain comprises SEQ ID NO:154, SEQ ID NO:155, or SEQ ID NO:156. In certain embodiments, the CD40 signaling fragment comprises, consists, or consists essentially of an SH3 motif (KPTNKAPH, PTNKAPHP or PTNKAPH), TRAF2 motif (PKQE, PKQET, PVQE, PVQET, SVQE, SVQET), TRAF6 motif (QEPQEINFP or QEPQEINFP), PKA motif (KKPTNKA, SRISVQE, or a combination thereof, or is a full length CD40 intracellular domain. In certain embodiments, one or more of the SH3, TRAF2, TRAF6, or PKA motifs of the CD40 signaling domain is mutated. In certain embodiments, one or more of the SH3, TRAF2, TRAF6, or PKA motifs of the CD40 signaling domain is present in multiple copies.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1— Schematic models for universal costimulatory proteins. (A) TCR incorporated antigen agnostic receptor (TIAAR) comprises modifying components of the TCR complex and associated signaling adaptors. (B) A constitutive costimulatory receptor comprising transmembrane domains (TMDs) and features that enable inducible or constitutive activation. (C) An inducible costimulatory receptor capable of induction and activation by extracellular ligand binding.

FIG. 2—Cytokine production by TCR incorporated antigen agnostic receptor (TIAAR) transduced cells. Cytokine production (Bcl-xL, IL2, IFNg and TNFa) from genetically modified and non-transduced T cells (NTD) of two donors was determined after overnight stimulation with either Ba/F3 OKT3 targets or left unstimulated (i.e., T cells only).

FIG. 3—Proliferation and activation marker expression by TIAAR transduced cells. Proliferation (T cell counts) and activation marker expression (41BB and CD69) was determined for genetically modified and non-transduced T cells (NTD) from donor 1 (3A) and donor 2 (3B) after 5-day co-culture with either Ba/F3 OKT3 targets or left unstimulated (i.e., T cells only).

FIG. 4—Cytokine production in leucine zipper based universal CoStAR (LZ) transduced cells. Cytokine production (Bcl-xL, IL2, IFNg and TNFa) from genetically modified and non-transduced T cells (NTD) of two donors was determined after overnight stimulation with either Ba/F3 OKT3 targets or left unstimulated (i.e., T cells only).

FIG. 5—Proliferation and activation marker expression by LZ-CoStAR transduced cells. Proliferation (T cell counts) and activation marker expression (41BB and CD69) was determined for genetically modified and non-transduced T cells (NTD) from donor 1 (5A) and donor 2 (5B) after 5-day co-culture with either Ba/F3 OKT3 targets or left unstimulated (i.e., T cells only).

FIG. 6—Cytokine production in inducible universal CoStAR transduced cells. Cytokine production (Bcl-xL, IL2, IFNg and TNFa) from genetically modified and non-transduced T cells (NTD) of two donors was determined after overnight stimulation with either Ba/F3 OKT3 targets or left unstimulated (i.e., T cells only). The universal CoStAR is inducible by pembrolizumab.

FIG. 7—Proliferation and activation marker expression by inducible universal CoStAR transduced cells. Proliferation (T cell counts) and activation marker expression (41BB and CD69) was determined for genetically modified and non-transduced T cells (NTD) from donor 1 (7A) and donor 2 (7B) after 5-day co-culture with either Ba/F3 OKT3 targets or left unstimulated (i.e., T cells only). The universal CoStAR is inducible by pembrolizumab.

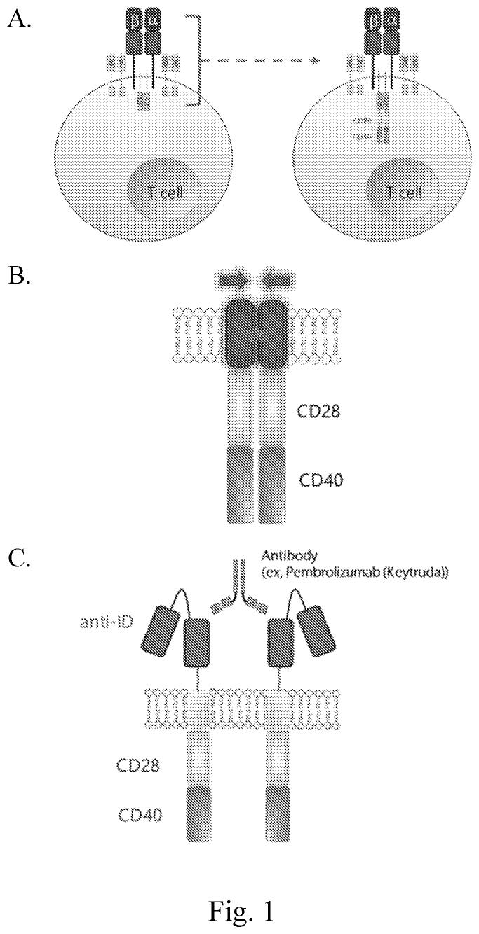

### DETAILED DESCRIPTION OF THE INVENTION

As used herein, “full length protein” or “full length receptor” refers to a receptor protein, such as, for example, a CD40 receptor protein. The term “full length” encompasses receptor proteins lacking up to about 5 or up to 10 amino acids, for example 1, 2, 3, 4, 5, 6, 7, 8, 9, or 10 amino acids, at the N-terminal of the mature receptor protein once its signal peptide has been cleaved. For instance, while a specific cleavage site of a receptors N-terminal signal peptide may be defined, variability in exact point of cleavage has been observed. The term “full length” does not imply presence or absence of amino acids of the receptors N-terminal signal peptide. In one embodiment, the term “full length” (e.g. a full length CD28 or a full length CD40 intracellular domain, according to certain aspects of the invention) encompasses mature receptor proteins (e.g. CD28 according to certain aspects of the invention) lacking the N terminal signal peptide lacking up to about 5, for example 1, 2, 3, 4, 5, or up to 10 amino acids at the N-terminal of the mature receptor protein once its signal peptide has been cleaved. As mentioned above, a “full length” CD28 receptor or other receptor or TCR clustering domain according to the various aspects of the invention does not include the signal peptide and may lack up to about 5, for example 1, 2, 3, 4, 5, or up to 10 amino acids at the N-terminal of the mature receptor protein (e.g. N terminal residues N, K, I, L and/or V). This is shown in the exemplary fusions, e.g. SEQ ID Nos. 4-12 (note that these may lack up to about 5, for example 1, 2, 3, 4, 5, or up to 10 amino acids at the N-terminal of the mature receptor protein as shown in the boxed region).

The chimeric protein of the present invention may comprise a TCR clustering domain as well as a signaling domain that advantageously may comprise a CD40 intracellular domain.

The TCR clustering domain may advantageously comprise one or more protein typically found in a TCR complex. Advantageously the TCR clustering domain may comprise CD3D, CD3E, CD3G or CD3Z or a portion thereof.

The CD3D antigen, delta polypeptide (TiT3 complex) encoded by the CD3D gene is part of the T-cell receptor/CD3 complex (TCR/CD3 complex) and is involved in T-cell development and signal transduction. The encoded membrane protein represents the delta subunit of the CD3 complex, and along with four other CD3 subunits, binds either TCR alpha/beta or TCR gamma/delta to form the TCR/CD3 complex on the surface of T-cells. Defects in this gene are a cause of severe combined immunodeficiency autosomal recessive T-cell-negative/B-cell-positive/NK-cell-positive (SCIDBNK). Two transcript variants encoding different isoforms have been found for this gene. Other variants may also exist, but the full-length natures of their transcripts has yet to be defined. See, e.g., annotation release 109.20200522 Assembly GRCh38.p13 (GRCh38.p13).

The CD3E antigen, epsilon polypeptide (TiT3 complex) encoded by the CD3E gene is the CD3-epsilon polypeptide, which together with CD3-gamma, -delta and -zeta, and the T-cell receptor alpha/beta and gamma/delta heterodimers, forms the T cell receptor-CD3 complex. This complex plays an important role in coupling antigen recognition to several intracellular signal-transduction pathways. The genes encoding the epsilon, gamma and delta polypeptides are located in the same cluster on chromosome 11. The epsilon polypeptide plays an essential role in T-cell development. See, e.g., accession numbers NM_000733 and NM_007648 for the mRNA sequences and accession numbers NP_000724 and NP_031674 for the protein sequences.

The CD3G antigen, gamma polypeptide (TiT3 complex) is encoded by the CD3G gene. CD3G (gamma chain) is one of the four peptides (gamma, delta, epsilon and zeta) that form CD3. Defects in CD3G are associated with T cell immunodeficiency. See, e.g., accession numbers NM_000073 and NM_009850 for the mRNA sequences and accession numbers NP_000064 and NP_033980 for the protein sequences.

The CD3Z antigen, zeta polypeptide (TiT3 complex) is encoded by the CD3Z. The glycoprotein CD3 zeta chain also known as T-cell receptor T3 zeta chain or CD247 (Cluster of Differentiation 247). T-cell receptor zeta (ζ), together with T-cell receptor alpha/beta and gamma/delta heterodimers and CD3-gamma, -delta, and -epsilon, forms the T-cell receptor-CD3 complex. The zeta chain plays an important role in coupling antigen recognition to several intracellular signal-transduction pathways. Low expression of the antigen results in impaired immune response. Two alternatively spliced transcript variants encoding distinct isoforms have been found for this gene. CD247 has been shown to interact with Janus kinase 3 and Protein unc-119 homolog. See, e.g., accession numbers NM_000734, NM_198053, NM_001378515, NM_001378516, NM_001113391, NM_001113392, NM_001113393, NM_001113394 and NM_031162 for the mRNA sequences and accession numbers NP_000725, NP_932170, NP_001365444, NP_001365445, NP_112439.1, NP_001106862.1, NP_001106862, NP_001106863 and NP_001106864 for the protein sequences.

CD3 eta (η) is an alternatively spliced product of the gene that encodes CD3 ζ (Clayton et al. 1991 Proceedings of the National Academy of Sciences of the USA 88, 5202-5206) and has an apparent molecular weight of 23 kDa (Orloff et al. 1989 Journal of Biological Chemistry 264, 14812-14817). CD3 ζ is predominantly found as a homodimer, but a fraction (approximately 10%) of CD3 ζ is found complexed as a heterodimer with CD3 η and in this form is thought to confer different signaling properties (Orloff et al. 1989 Journal of Biological Chemistry 264, 14812-14817).

The TCR clustering domain may comprise the constant chains of TCR alpha (TCRα), TCR beta (TCR β), TCR gamma (TCRγ) or TCR delta (TCR δ) or a portion thereof. Representative locus information is provided below:

T-cell receptor alpha locus
Identifiers

| Symbol | TRA |
|---|---|
| Alt. symbols | TCRA, TRA@ |
| NCBI gene | 6955 |
| HGNC | 12027 |
| OMIM | 186880 |

Other data

| | |
|---|---|
| Locus | Chr. 14 q11.2 |

T-cell receptor beta locus
Identifiers

| Symbol | TRB |
|---|---|
| Alt. symbols | TCRB, TRB@ |
| NCBI gene | 6957 |
| HGNC | 12155 |
| OMIM | 186930 |

Other data

| | |
|---|---|
| Locus | Chr. 7 q34 |

T-cell receptor delta locus
Identifiers

| Symbol | TRD |
|---|---|
| Alt. symbols | TCRD, TRD@, TCRDV1 |
| NCBI gene | 6964 |
| HGNC | 12252 |

Other data

| | |
|---|---|
| Locus | Chr. 14 q11.2 |

T-cell receptor gamma locus
Identifiers

| Symbol | TRG |
|---|---|
| Alt. symbols | TCRG, TRG@ |
| NCBI gene | 6965 |
| HGNC | 12271 |

Other data

| | |
|---|---|
| Locus | Chr. 7 p14 |

The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

Costimulatory receptor proteins useful in the chimeric proteins of the invention include, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6, which in their natural form comprise extracellular ligand binding domains and intracellular signal transducing domains. For example, CD2 is characterized as a cell adhesion molecule found on the surface of T cells and is capable of initiating intracellular signals necessary for T cell activation. CD27 is characterized as a type II transmembrane glycoprotein belonging to the TNFR superfamily (TNFRSF) whose expression on B cells is induced by antigen-receptor activation in B cells. CD28 is one of the proteins on T cells and is the receptor for CD80 (B7.1) and CD86 (B7.2) ligands on antigen-presenting cells. CD137 (4-1BB) ligand is found on most leukocytes and on some non-immune cells. OX40 ligand is expressed on many antigen-presenting cells such as DC2s (dendritic cells), macrophages, and B lymphocytes. In one embodiment, the costimulatory receptor protein is full length CD28 as defined herein.

CD40 is a member of the tumor necrosis factor receptor (TNFR) superfamily and several isoforms are generated by alternative splicing. Its ligand, CD154 (also called CD40L) is a protein that is primarily expressed on activated T cells. For reference, the human CD40 isoform 1 protein sequence is set forth in GenBank accession No. NP_001241.1, including signal peptide (amino acids 1-20), transmembrane domain (amino acids 194-215), and cytoplasmic domain (amino acids 216-277)(SEQ ID NO:22). CD40 receptor signaling involves adaptor proteins including but not limited to TNF receptor—associated factors (TRAF), and the cytoplasmic domain comprises signaling components, including but not limited to an SH3 motif (KPTNKAPH), TRAF2 motif (PKQE, PVQE, SVQE), TRAF6 motif (QEPQEINFP) and PKA motif (KKPTNKA, SRISVQE). Further motifs for binding to TRAF1, TRAF2, TRAF3, and TRAF5 comprise the major consensus sequence (P/S/A/T)X(Q/E)E or minor consensus sequence PXQXXD and can be identified in or obtained from, without limitation, TNFR family members such as CD30, Ox40, 4-1BB, and the EBV oncoprotein LMP1. (See, e.g., Ye, H et al., The Structural Basis for the Recognition of Diverse Receptor Sequences by TRAF2. Molecular Cell, 1999; 4(3):321-30. doi: 10.1016/S1097-2765(00)80334-2; Park H H, Structure of TRAF Family: Current Understanding of Receptor Recognition. Front. Immunol. 2018; 9:1999. doi: 10.3389/fimmu.2018. 01999).

Examples disclosed herein demonstrate operation of CD40 as a signaling domain and further that cytokine and chemokine expression profiles are altered by signaling domain selection. In this regard, the CD40 signaling domains of the invention provide distinct and overlapping responses induced by the different factor binding sites. (See, e.g., Ahonen, C L et al., The CD40-TRAF6 axis controls affinity maturation and the generation of long-lived plasma cells. Nat Immunol. 2002; 3: 451-456; Mackey M F et al., Distinct contributions of different CD40 TRAF binding sites to CD154-induced dendritic cell maturation and IL-12 secretion. Eur J Immunol. 2003; 33: 779-789; Mukundan Let al., TNF receptor-associated factor 6 is an essential mediator of CD40-activated proinflammatory pathways in monocytes and macrophages. J Immunol. 2005; 174: 1081-1090.

In certain embodiments, a chimeric protein of the invention comprises substantially all of a CD40 costimulatory domain. In certain embodiments, a chimeric protein of the invention comprises two or more CD40 costimulatory domains. In certain embodiments, a chimeric protein of the invention comprises a CD40 costimulatory domain signaling component or motif, including but not limited to an SH3 motif (KPTNKAPH), TRAF2 motif (PKQE, PVQE, SVQE), TRAF3 motif, TRAF6 motif (QEPQEINFP) or PKA motif (KKPTNKA, SRISVQE) as well as two or more, or three or more, or four or more such components of motifs, which can be in multiple copies and arranged in any order. In certain embodiments, a chimeric protein of the invention comprises a CD40 costimulatory domain and a CD40 costimulatory domain signaling component or motif.

In certain embodiments, selection of one or more costimulatory domain signaling component or motif is guided by the cell in which the chimeric protein is to be expressed and/or a desired costimulatory activity more closely identified with a signaling component or motif, or avoidance of a costimulatory activity more closely identified with a signaling component or motif.

In certain embodiments, a chimeric protein signaling domain comprises, in addition to a CD40 costimulatory domain or signaling component or motif thereof, or two or more such domains or components or motifs or combinations thereof, an additional full length costimulatory domain or signaling component thereof from, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6,

For reference, the human CD28 protein sequence is set forth in GenBank accession No. NP_006130.1, including signal peptide (amino acids 1-18), extracellular domain (amino acids 19-152), transmembrane domain (amino acids 153-179) and cytoplasmic domain (amino acids 180-200). The extracellular domain includes an immunoglobulin type domain (amino acids 21-136) which contains amino acids with compose the antigen binding site and amino acids that form the homodimer interface. The extracellular domain includes several asparagine residues which may be glycosylated, and the intracellular domain comprises serine and tyrosine residues, which may be phosphorylated.

For reference, the human CD8 alpha chain protein sequence is set forth by GenBank accession No. NP_001139345.1, including signal peptide (amino acids 1-21), extracellular domain (amino acids 22-182), transmembrane domain (amino acids 183-203), and cytoplasmic domain (amino acids 204-235). The extracellular domain includes an immunoglobulin type domain (amino acids 28-128) which contains amino acids with compose the antigen binding site and amino acids that form the homodimer interface. The extracellular domain includes several asparagine residues which may be glycosylated, and the intracellular domain comprises serine and tyrosine residues, which may be phosphorylated.

For reference, the human IgG4 constant region sequence is set forth in UniProtKB/Swiss-Prot: accession No. P01861.1, including CH1 (amino acids 1-98), hinge (amino acids 99-110), CH2 (amino acids 111-220), CH3 (amino acids 221-327). The CH2 region includes asparagine at amino acid 177, which is the glycosylated and associated with Fc receptor and antibody-dependent cell-mediated cytotoxicity (ADCC).

For reference, the protein sequence of human CD137 (41BB), another TNFR superfamily member, is set forth by GenBank accession No. NP_001552.2, including signal peptide (amino acids 1-23), extracellular domain (amino acids 24-186), transmembrane domain (amino acids 187-213), and cytoplasmic domain (amino acids 214-255).

For reference, the human CD134 (OX40) protein sequence is set forth by GenBank accession No. NP_003318.1, including signal peptide (amino acids 1-28), extracellular domain (amino acids 29-214), transmembrane domain (amino acids 215-235), and cytoplasmic domain (amino acids 236-277). This receptor has been shown to activate NF-kappaB through its interaction with adaptor proteins TRAF2 and TRAF5 and studies suggest that this receptor promotes expression of apoptosis inhibitors BCL2 and BCL21L1/BCL2-XL.

The human T-cell surface antigen CD2 has at least two isoforms. For reference, the human CD2 isoform1 protein sequence is set forth by NP_001315538.1, including signal peptide (amino acids 1-24), extracellular domain (amino acids 25-235), transmembrane domain (amino acids 236-261), and cytoplasmic domain (amino acids 262-377). The human CD2 isoform2 protein sequence is set forth by NP_001758.2

For reference, the human CD357 (GITR) isoform-1 protein sequence is set forth by GenBank accession No. NP_004186.1, including signal peptide (amino acids 1-25), extracellular domain (amino acids 26-162), transmembrane domain (amino acids 163-183), and cytoplasmic domain (amino acids 184-241).

For reference, the human CD29 (beta1 integrin) protein sequence is set forth by GenBank accession No. NP_596867, including signal peptide (amino acids 1-20), extracellular domain (amino acids 21-728), transmembrane domain (amino acids 729-751), and cytoplasmic domain (amino acids 752-798).

The human CD150 (SLAM) protein sequence has at several isoforms. In addition to the transmembrane form of CD150 (mCD150), cells of hematopoietic lineage express mRNA encoding the secreted form of CD150 (sCD150), which lacks the entire transmembrane region of 30 amino acids. For reference, human SLAM isoform b is set forth by GenBank accession No. NP_003028.1, including signal peptide (amino acids 1-20), extracellular domain (amino acids 21-237), transmembrane domain (amino acids 238-258), and cytoplasmic domain (amino acids 259-335). Human SLAM isoform a is set forth by GenBank accession No. NP_001317683.1.

In embodiments of the invention, a chimeric protein may be expressed alone under the control of a promoter in a therapeutic population of cells that have therapeutic activity, for example, Tumor Infiltrating Lymphocytes (TILs). Alternatively, the chimeric protein may be expressed along with a therapeutic transgene such as a chimeric antigen receptor (CAR) and/or T-cell Receptor (TCR). Suitable TCRs and CARs are well known in the literature, for example HLA-A*02-NYESO-1 specific TCRs (Rapoport et al. Nat Med 2015) or anti-CD19scFv.CD3ζ fusion CARs (Kochenderfer et al. J Clin Oncol 2015) which have been successfully used to treat Myeloma or B-cell malignancies respectively. The chimeric proteins described herein may be expressed with any known CAR or TCR thus providing the cell with a regulatable growth switch to allow cell expansion in-vitro or in-vivo, and a conventional activation mechanism in the form of the TCR or CAR for anti-cancer activity. Thus the invention provides a cell for use in adoptive cell therapy comprising a chimeric protein as described herein and a TCR and/or CAR that specifically binds to a tumor associated antigen. An exemplary chimeric protein comprising CD28 includes an extracellular antigen binding domain and an extracellular, transmembrane and intracellular signaling domain.

A chimeric protein of the invention optionally comprises a spacer region between the TCR clustering domain and the costimulatory receptor. As used herein, the term “spacer” refers to the extracellular structural region of a chimeric protein that separates the TCR clustering domain from the signaling domain of the chimeric protein. In certain embodiments long spacers are employed, for example to target membrane-proximal epitopes or glycosylated antigens (see Guest R. D. et al. The role of extracellular spacer regions in the optimal design of chimeric immune receptors: evaluation of four different scFvs and antigens. J. Immunother. 2005; 28:203-211; Wilkie S. et al., Retargeting of human T cells to tumor-associated MUC1: the evolution of a chimeric antigen receptor. J. Immunol. 2008; 180:4901-4909). In other embodiments, chimeric proteins bear short spacers, for example to target membrane distal epitopes (see Hudecek M. et al., Receptor affinity and extracellular domain modifications affect tumor recognition by ROR1-specific chimeric antigen receptor T cells. Clin. Cancer Res. 2013; 19:3153-3164; Hudecek M. et al., The nonsignalling extracellular spacer domain of chimeric antigen receptors is decisive for in vivo antitumor activity. Cancer Immunol. Res. 2015; 3:125-135). In certain embodiments, the spacer comprises all or part of or is derived from an IgG hinge, including but not limited to IgG1, IgG2, or IgG4. By “derived from an Ig hinge” is meant a spacer comprising insertions, deletions, or mutations in an IgG hinge. In certain embodiments, a spacer can comprise all or part of one or more antibody constant domains, such as but not limited to CH2 and/or CH3 domains. In certain embodiments, in a spacer comprising all or part of a CH2 domain, the CH2 domain is modified so as not to bind to an Fc receptor. For example, Fc receptor binding in myeloid cells has been found to impair CAR T cell functionality. In certain embodiments, the spacer comprises all or part of an Ig-like hinge from CD28, CD8, or other protein comprising a hinge region. In certain embodiments of the invention that comprise a spacer, the spacer is from 1 and 50 amino acids in length.

In certain embodiments, the chimeric protein extracellular domain comprises a linker. Linkers comprise short runs of amino acids used to connect domains, for example a binding domain with a spacer or transmembrane domain. In order for there to be flexibility to bind ligand, a ligand binding domain will usually be connected to a spacer or a transmembrane domain by flexible linker comprising from about 5 to 25 amino acids, such as, for example, AAAGSGGSG or GGGGSGGGGSGGGGS. In certain embodiments, a chimeric protein comprises a TCR clustering domain joined directly to a signaling domain by a linker, and without a spacer. In certain embodiments, a chimeric protein comprises a binding domain joined directly to a transmembrane by a spacer and without a linker.

As discussed above, in certain embodiments, a chimeric protein comprises a full length primary costimulatory receptor which can comprise an extracellular ligand binding and intracellular signaling portion of, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6. In other embodiments, the chimeric protein, for instance may comprise an extracellular ligand binding domain of one of the aforementioned proteins and an intracellular signaling domain of another of the aforementioned proteins. In certain embodiments, the signaling portion of the chimeric protein comprises a single signaling domain. In other embodiments, the signaling portion of the chimeric protein comprises a second intracellular signaling domain such as but not limited to: CD2, CD27, CD28, CD40, CD134 (OX40), CD137 (4-1BB), CD150 (SLAM). In certain embodiments, the first and second intracellular signaling domains are the same. In other embodiments, the first and second intracellular signaling domains are different. In certain embodiments, the costimulatory receptor is capable of dimerization. Without being bound by theory, it is thought that chimeric proteins dimerize or associate with other accessory molecules for signal initiation. In certain embodiments, chimeric proteins dimerize or associate with accessory molecules through transmembrane domain interactions. In certain embodiments, dimerization or association with accessory molecules is assisted by costimulatory receptor interactions in the intracellular portion, and/or the extracellular portion of the costimulatory receptor.

Although the main function of the transmembrane is to anchor the chimeric protein in the T cell membrane, in certain embodiments, the transmembrane domain influences chimeric protein function. In certain embodiments, the transmembrane domain is comprised by the full length primary costimulatory receptor domain. In embodiments of the invention wherein the chimeric protein construct comprises an extracellular domain of one receptor and an intracellular signaling domain of a second receptor, the transmembrane domain can be that of the extracellular domain or the intracellular domain. In certain embodiments, the transmembrane domain is from CD4, CD8a, CD28, or ICOS. Gueden et al. associated use of the ICOS transmembrane domain with increased CART cell persistence and overall anti-tumor efficacy (Guedan S. et al., Enhancing CART cell persistence through ICOS and 4-1BB costimulation. JCI Insight. 2018; 3:96976). In an embodiment, the transmembrane domain comprises a hydrophobic α helix that spans the cell membrane.

In some embodiments, amino acid sequence variants of the TCR clustering domain or other moieties provided herein are contemplated. For example, it may be desirable to improve the binding affinity and/or other biological properties of the moiety. Amino acid sequence variants of an antibody moiety may be prepared by introducing appropriate modifications into the nucleotide sequence encoding the clustering moiety, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics.

In some embodiments, TCR clustering domain moieties comprising one or more amino acid substitutions, deletions, or insertions are provided. Amino acid substitutions may be introduced into a binding domain of interest and the products screened for a desired activity, e.g., retained/improved clustering or decreased immunogenicity. In certain embodiments, amino acid substitutions may be introduced into one or more of the primary co-stimulatory receptor domain (extracellular or intracellular), secondary costimulatory receptor domain, or extracellular co-receptor domain. Accordingly, the invention encompasses chimeric proteins and component parts particularly disclosed herein as well as variants thereof, i.e. chimeric proteins and component parts having at least 75%, at least 80%, at least 85%, at least 87%, at least 90%, at least 91%, at least 92%, at least 93%, at least 94%, at least 95%, at least 96%, at least 97%, at least 98%, at least 99% sequence identity to the amino acid sequences particularly disclosed herein. The terms “percent similarity,” “percent identity,” and “percent homology” when referring to a particular sequence are used as set forth in the University of Wisconsin GCG software program BestFit. Other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448).

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, 5, 10, 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:

Conservative substitutions are shown in the Table below.

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucinne; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped into different classes according to common side-chain properties: a. hydrophobic: Norleucine, Met, Ala, Val, Leu, Ile; b. neutral hydrophilic: Cys, Ser, Thr, Asn, Gln; c. acidic: Asp, Glu; d. basic: His, Lys, Arg; e. residues that influence chain orientation: Gly, Pro; aromatic: Trp, Tyr, Phe. Non-conservative substitutions will entail exchanging a member of one of these classes for another class.

The cells used in the present invention may be any lymphocyte that is useful in adoptive cell therapy, such as a T-cell or a natural killer (NK) cell, an NKT cell, a gamma/delta T-cell or T regulatory cell. The cells may be allogeneic or autologous to the patient.

T cells or T lymphocytes are a type of lymphocyte that have a central role in cell-mediated immunity. They can be distinguished from other lymphocytes, such as B cells and natural killer cells (NK cells), by the presence of a T-cell receptor (TCR) on the cell surface. There are various types of T cell, as summarized below. Cytotoxic T cells (TC cells, or CTLs) destroy virally infected cells and tumor cells, and are also implicated in transplant rejection. CTLs express the CD8 molecule at their surface.

These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO. Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells. Naturally occurring Treg cells (also known as CD4⁺CD25⁺FoxP3⁺Treg cells) arise in the thymus and have been linked to interactions between developing T cells with both myeloid (CD11c⁺) and plasmacytoid (CD123⁺) dendritic cells that have been activated with TSLP. Naturally occurring Treg cells can be distinguished from other T cells by the presence of an intracellular molecule called FoxP3. Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MEW independent manner. NK cells (belonging to the group of innate lymphoid cells) are defined as large granular lymphocytes (LGL) and constitute the third kind of cells differentiated from the common lymphoid progenitor generating B and T lymphocytes.

In certain embodiments, therapeutic cells of the invention comprise autologous cells engineered to express a chimeric protein. In certain embodiments, therapeutic cells of the invention comprise allogeneic cells engineered to express a chimeric protein. Autologous cells expressing chimeric proteins may be advantageous in avoiding graft-versus-host disease (GVHD) due to TCR-mediated recognition of recipient alloantigens.

An aspect of the invention provides a nucleic acid sequence of the invention, encoding any of the chimeric proteins, polypeptides, or proteins described herein (including functional portions and functional variants thereof). As used herein, the terms “polynucleotide”, “nucleotide”, and “nucleic acid” are intended to be synonymous with each other. It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed, e.g. codon optimization. Nucleic acids according to the invention may comprise DNA or RNA. They may be single stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid sequence may encode the protein sequence shown as SEQ ID NO:2 or a variant thereof. The nucleotide sequence may comprise a codon optimized nucleic acid sequence shown engineered for expression in human cells.

The invention also provides a nucleic acid sequence which comprises a nucleic acid sequence encoding a chimeric protein and a further nucleic acid sequence encoding a T-cell receptor (TCR) and/or chimeric antigen receptor (CAR).

The nucleic acid sequences may be joined by a sequence allowing co-expression of the two or more nucleic acid sequences. For example, the construct may comprise an internal promoter, an internal ribosome entry sequence (IRES) sequence or a sequence encoding a cleavage site. The cleavage site may be self-cleaving, such that when the polypeptide is produced, it is immediately cleaved into the discrete proteins without the need for any external cleavage activity. Various self-cleaving sites are known, including the Foot- and Mouth disease virus (FMDV) and the 2A self-cleaving peptide. The co-expressing sequence may be an internal ribosome entry sequence (IRES). The co-expressing sequence may be an internal promoter.

In an aspect, the present invention provides a vector which comprises a nucleic acid sequence or nucleic acid construct of the invention.

Such a vector may be used to introduce the nucleic acid sequence(s) or nucleic acid construct(s) into a host cell so that it expresses one or more chimeric protein(s) according to the first aspect of the invention and, optionally, one or more other proteins of interest (POI), for example a TCR or a CAR. The vector may, for example, be a plasmid or a viral vector, such as a retroviral vector or a lentiviral vector, or a transposon-based vector or synthetic mRNA.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Vectors derived from retroviruses, such as the lentivirus, are suitable tools to achieve long-term gene transfer since they allow long-term, stable integration of a transgene or transgenes and its propagation in daughter cells. The vector may be capable of transfecting or transducing a lymphocyte including a T cell or an NK cell. The present invention also provides vectors in which a nucleic acid of the present invention is inserted. The expression of natural or synthetic nucleic acids encoding a chimeric protein, and optionally a TCR or CAR is typically achieved by operably linking a nucleic acid encoding the chimeric protein and TCR/CAR polypeptide or portions thereof to one or more promoters, and incorporating the construct into an expression vector.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, MSCV promoter, MND promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

The vectors can be suitable for replication and integration in eukaryotic cells. Typical cloning vectors contain transcription and translation terminators, initiation sequences, and promoters useful for regulation of the expression of the desired nucleic acid sequence. Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193). In some embodiments, the constructs expressed are as shown in SEQ ID NOS:32-65 and 67-79. In some embodiments the nucleic acids are multi-cistronic constructs that permit the expression of multiple transgenes (e.g., chimeric protein and a TCR and/or CAR etc.) under the control of a single promoter. In some embodiments, the transgenes (e.g., chimeric protein and a TCR and/or CAR etc.) are separated by a self-cleaving 2A peptide. Examples of 2A peptides useful in the nucleic acid constructs of the invention include F2A, P2A, T2A and E2A. In other embodiments of the invention, the nucleic acid construct of the invention is a multi-cistronic construct comprising two promoters; one promoter driving the expression of chimeric protein and the other promoter driving the expression of the TCR or CAR. In some embodiments, the dual promoter constructs of the invention are uni-directional. In other embodiments, the dual promoter constructs of the invention are bi-directional. In order to assess the expression of the chimeric protein polypeptide or portions thereof, the expression vector to be introduced into a cell can also contain either a selectable marker gene or a reporter gene or both to facilitate identification and selection of expressing cells from the population of cells sought to be transfected or transduced through viral vectors.

Prior to expansion and genetic modification, a source of cells (e.g., immune effector cells, e.g., T cells or NK cells) is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In one aspect, T cells are isolated from peripheral blood lymphocytes by lysing the red blood cells and depleting the monocytes, for example, by centrifugation through a PERCOLL™ gradient or by counterflow centrifugal elutriation.

In another aspect, Tumor infiltrating cells (TILs) are isolated and/or expanded from a tumor, for example by a fragmented, dissected, or enzyme digested tumor biopsy or mass.

A specific subpopulation of T cells, such as CD3+, CD28+, CD4+, CD8+, CD45RA+, and CD45RO+T cells, can be further isolated by positive or negative selection techniques. For example, in one aspect, T cells are isolated by incubation with anti-CD3/anti-CD28-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD16, HLA-DR, and CD8. In certain aspects, it may be desirable to enrich for or positively select for regulatory T cells which typically express CD4+, CD25+, CD62Lhi, GITR+, CD137, PD1, TIM3, LAG-3, CD150 and FoxP3+. Alternatively, in certain aspects, T regulatory cells are depleted by anti-CD25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

A specific subpopulation of chimeric protein effector cells that specifically bind to a target antigen can be enriched for by positive selection techniques. For example, in some embodiments, effector cells are enriched for by incubation with target antigen-conjugated beads for a time period sufficient for positive selection of the desired abTCR effector cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of effector cells present at low levels in the heterogeneous cell population, use of longer incubation times, such as 24 hours, can increase cell yield. Longer incubation times may be used to isolate effector cells in any situation where there are few effector cells as compared to other cell types. The skilled artisan would recognize that multiple rounds of selection can also be used in the context of this invention.

T cells for stimulation can also be frozen after a washing step. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In embodiments described herein, the immune effector cell can be an allogeneic immune effector cell, e.g., T cell or NK cell.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some aspects, downregulation of HLA may be accomplished by reducing or eliminating expression of beta-2 microglobulin (B2M).

In some embodiments, the T cell can lack a functional TCR and a functional HLA, e.g., HLA class I and/or HLA class II. Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. a cell engineered by any method described herein. For example, the cell can be a cell that does not express or expresses at low levels an inhibitory molecule, e.g., that can decrease the ability of a chimeric protein-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, Ga19, adenosine, and TGFR beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, expansion can be performed using flasks or containers, or gas-permeable containers known by those of skill in the art and can proceed for 7 days, 8 days, 9 days, 10 days, 11 days, 12 days, 13 days, or 14 days, about 7 days to about 14 days, about 8 days to about 14 days, about 9 days to about 14 days, about 10 days to about 14 days, about 11 days to about 14 days, about 12 days to about 14 days, or about 13 days to about 14 days. In some embodiments, the second TIL expansion can proceed for about 14 days.

In certain embodiments, the expansion can be performed using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UHCT-1 (commercially available from BioLegend, San Diego, Calif., USA). Chimeric protein cells can be expanded in vitro by including one or more antigens, including antigenic portions thereof, such as epitope(s), of a cancer, which can be optionally expressed from a vector, such as a human leukocyte antigen A2 (HLA-A2) binding peptide, e.g., 0.3.mu.M MART-1:26-35 (27L) or gp100:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. Chimeric protein cells may also be rapidly expanded by re-stimulation with the same antigen(s) of the cancer pulsed onto HLA-A2-expressing antigen-presenting cells. Alternatively, the chimeric protein cells can be further stimulated with, e.g., example, irradiated, autologous lymphocytes or with irradiated HLA-A2+ allogeneic lymphocytes and IL-2. In some embodiments, the stimulation occurs as part of the expansion. In some embodiments, the expansion occurs in the presence of irradiated, autologous lymphocytes or with irradiated HLA-A2+ allogeneic lymphocytes and IL-2.

In certain embodiments, the cell culture medium comprises IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL, or between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In certain embodiments, the cell culture medium comprises OKT3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, about 1 μg/mL or between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT3 antibody.

In certain embodiments, a combination of IL-2, IL-7, IL-15, and/or IL-21 are employed as a combination during the expansion. In some embodiments, IL-2, IL-7, IL-15, and/or IL-21 as well as any combinations thereof can be included during the expansion. In some embodiments, a combination of IL-2, IL-15, and IL-21 are employed as a combination during the expansion. In some embodiments, IL-2, IL-15, and IL-21 as well as any combinations thereof can be included.

In certain embodiments, the expansion can be conducted in a supplemented cell culture medium comprising IL-2, OKT-3, and antigen-presenting feeder cells.

In certain embodiments, the expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15, or about 500 IU/mL of IL-15 to about 100 IU/mL of IL-15, or about 400 IU/mL of IL-15 to about 100 IU/mL of IL-15 or about 300 IU/mL of IL-15 to about 100 IU/mL of IL-15 or about 200 IU/mL of IL-15, or about 180 IU/mL of IL-15.

In some embodiments, the expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21, or about 20 IU/mL of IL-21 to about 0.5 IU/mL of IL-21, or about 15 IU/mL of IL-21 to about 0.5 IU/mL of IL-21, or about 12 IU/mL of IL-21 to about 0.5 IU/mL of IL-21, or about 10 IU/mL of IL-21 to about 0.5 IU/mL of IL-21, or about 5 IU/mL of IL-21 to about 1 IU/mL of IL-21, or about 2 IU/mL of IL-21. In some embodiments, the cell culture medium comprises about 1 IU/mL of IL-21, or about 0.5 IU/mL of IL-21.

In some embodiments the antigen-presenting feeder cells (APCs) are PBMCs. In an embodiment, the ratio of chimeric protein cells to PBMCs and/or antigen-presenting cells in the expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500, or between 1 to 50 and 1 to 300, or between 1 to 100 and 1 to 200.

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle:cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

Viral- and non-viral-based genetic engineering tools can be used to generate chimeric protein cells, including without limitation T cells, NK cells resulting in permanent or transient expression of therapeutic genes. Retrovirus-based gene delivery is a mature, well-characterized technology, which has been used to permanently integrate CARs into the host cell genome (Scholler J., e.g. Decade-long safety and function of retroviral-modified chimeric antigen receptor T cells. Sci. Transl. Med. 2012; 4:132ra53; Rosenberg S. A. et al., Gene transfer into humans—immunotherapy of patients with advanced melanoma, using tumor-infiltrating lymphocytes modified by retroviral gene transduction. N. Engl. J. Med. 1990; 323:570-578)

Non-viral DNA transfection methods can also be used. For example, Singh et al describes use of the Sleeping Beauty (SB) transposon system developed to engineer CAR T cells (Singh H., et al., Redirecting specificity of T-cell populations for CD19 using the Sleeping Beauty system. Cancer Res. 2008; 68:2961-2971) and is being used in clinical trials (see e.g., ClinicalTrials.gov: NCT00968760 and NCT01653717). The same technology is applicable to engineer chimeric protein cells.

Multiple SB enzymes have been used to deliver transgenes. Mates describes a hyperactive transposase (SB100X) with approximately 100-fold enhancement in efficiency when compared to the first-generation transposase. SB100X supported 35-50% stable gene transfer in human CD34(+) cells enriched in hematopoietic stem or progenitor cells. (Mates L. et al., Molecular evolution of a novel hyperactive Sleeping Beauty transposase enables robust stable gene transfer in vertebrates. Nat. Genet. 2009; 41:753-761) and multiple transgenes can be delivered from multicistronic single plasmids (e.g., Thokala R. et al., Redirecting specificity of T cells using the Sleeping Beauty system to express chimeric antigen receptors by mix-and-matching of VL and VH domains targeting CD123+ tumors. PLoS ONE. 2016; 11:e0159477) or multiple plasmids (e.g., Hurton L. V. et al., Tethered IL-15 augments antitumor activity and promotes a stem-cell memory subset in tumor-specific T cells. Proc. Natl. Acad. Sci. USA. 2016; 113:E7788-E7797). Such systems are used with chimeric proteins of the invention.

Morita et al, describes the piggyBac transposon system to integrate larger transgenes (Morita D. et al., Enhanced expression of anti-CD19 chimeric antigen receptor in piggyBac transposon-engineered T cells. Mol. Ther. Methods Clin. Dev. 2017; 8:131-140) Nakazawa et al. describes use of the system to generate EBV-specific cytotoxic T-cells expressing HER2-specific chimeric antigen receptor (Nakazawa Y et al, PiggyBac-mediated cancer immunotherapy using EBV-specific cytotoxic T-cells expressing HER2-specific chimeric antigen receptor. Mol. Ther. 2011; 19:2133-2143). Manuri et al used the system to generate CD-19 specific T cells (Manuri P. V. R. et al., piggyBac transposon/transposase system to generate CD19-specific T cells for the treatment of B-lineage malignancies. Hum. Gene Ther. 2010; 21:427-437).

Transposon technology is easy and economical. One potential drawback is the longer expansion protocols currently employed may result in T cell differentiation, impaired activity and poor persistence of the infused cells. Monjezi et al describe development minicircle vectors that minimize these difficulties through higher efficiency integrations (Monjezi R. et al., Enhanced CAR T-cell engineering using non-viral Sleeping Beauty transposition from minicircle vectors. Leukemia. 2017; 31:186-194). These transposon technologies can be used for chimeric proteins of the invention.

The present invention also relates to a pharmaceutical composition containing a vector or a chimeric protein expressing cell of the invention together with a pharmaceutically acceptable carrier, diluent or excipient, and optionally one or more further pharmaceutically active polypeptides and/or compounds.

In some embodiments, a pharmaceutical composition is provided comprising a chimeric protein described above and a pharmaceutically acceptable carrier. In some embodiments, a pharmaceutical composition is provided comprising a nucleic acid encoding a chimeric protein according to any of the embodiments described above and a pharmaceutically acceptable carrier. In some embodiments, a pharmaceutical composition is provided comprising an effector cell expressing a chimeric protein described above and a pharmaceutically acceptable carrier. Such a formulation may, for example, be in a form suitable for intravenous infusion.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An aspect of the invention provides a population of modified T cells expressing a recombinant chimeric protein. A suitable population may be produced by a method described above.

The population of modified T cells may be for use as a medicament. For example, a population of modified T cells as described herein may be used in cancer immunotherapy therapy, for example adoptive T cell therapy.

Other aspects of the invention provide the use of a population of modified T cells as described herein for the manufacture of a medicament for the treatment of cancer, a population of modified T cells as described herein for the treatment of cancer, and a method of treatment of cancer may comprise administering a population of modified T cells as described herein to an individual in need thereof.

The population of modified T cells may be autologous i.e. the modified T cells were originally obtained from the same individual to whom they are subsequently administered (i.e. the donor and recipient individual are the same). A suitable population of modified T cells for administration to the individual may be produced by a method comprising providing an initial population of T cells obtained from the individual, modifying the T cells to express a cAMP PDE or fragment thereof and an antigen receptor which binds specifically to cancer cells in the individual, and culturing the modified T cells.

The population of modified T cells may be allogeneic i.e. the modified T cells were originally obtained from a different individual to the individual to whom they are subsequently administered (i.e. the donor and recipient individual are different). The donor and recipient individuals may be HLA matched to avoid GVHD and other undesirable immune effects. A suitable population of modified T cells for administration to a recipient individual may be produced by a method comprising providing an initial population of T cells obtained from a donor individual, modifying the T cells to express a chimeric protein which binds specifically to cancer cells in the recipient individual, and culturing the modified T cells.

Following administration of the modified T cells, the recipient individual may exhibit a T cell mediated immune response against cancer cells in the recipient individual. This may have a beneficial effect on the cancer condition in the individual.

Cancer conditions may be characterized by the abnormal proliferation of malignant cancer cells and may include leukaemias, such as AML, CIVIL, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumor may be immunogenic). For example, the cancer cells may be capable of eliciting a systemic immune response in the individual against one or more antigens expressed by the cancer cells. The tumor antigens that elicit the immune response may be specific to cancer cells or may be shared by one or more normal cells in the individual.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

The term “therapeutically effective amount” refers to an amount of a chimeric protein or composition comprising a chimeric protein as disclosed herein, effective to “treat” a disease or disorder in an individual. In the case of cancer, the therapeutically effective amount of a chimeric protein or composition comprising a chimeric protein as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent a chimeric protein or composition comprising a chimeric protein as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that improves progression free survival of a patient. In the case of infectious disease, such as viral infection, the therapeutically effective amount of a chimeric protein or composition comprising a chimeric protein as disclosed herein can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

Cells, including T and NK cells, expressing chimeric proteins for use in the methods of the present may either be created ex vivo either from a patient's own peripheral blood (autologous), or in the setting of a haematopoietic stem cell transplant from donor peripheral blood (allogenic), or peripheral blood from an unconnected donor (allogenic). Alternatively, T-cells or NK cells may be derived from ex-vivo differentiation of inducible progenitor cells or embryonic progenitor cells to T-cells or NK cells. In these instances, T-cells expressing a chimeric protein and, optionally, a CAR and/or TCR, are generated by introducing DNA or RNA coding for the chimeric protein and, optionally, a CAR and/or TCR, by one of many means including transduction with a viral vector, transfection with DNA or RNA.

T or NK cells expressing a chimeric protein of the present invention and, optionally, expressing a TCR and/or CAR may be used for the treatment of haematological cancers or solid tumors.

A method for the treatment of disease relates to the therapeutic use of a vector or cell, including a T or NK cell, of the invention. In this respect, the vector, or T or NK cell may be administered to a subject having an existing disease or condition in order to lessen, reduce or improve at least one symptom associated with the disease and/or to slow down, reduce or block the progression of the disease. The method of the invention may cause or promote T-cell mediated killing of cancer cells. The vector, or T or NK cell according to the present invention may be administered to a patient with one or more additional therapeutic agents. The one or more additional therapeutic agents can be co-administered to the patient. By “co-administering” is meant administering one or more additional therapeutic agents and the vector, or T or NK cell of the present invention sufficiently close in time such that the vector, or T or NK cell can enhance the effect of one or more additional therapeutic agents, or vice versa. In this regard, the vectors or cells can be administered first and the one or more additional therapeutic agents can be administered second, or vice versa. Alternatively, the vectors or cells and the one or more additional therapeutic agents can be administered simultaneously. One co-administered therapeutic agent that may be useful is IL-2, as this is currently used in existing cell therapies to boost the activity of administered cells. However, IL-2 treatment is associated with toxicity and tolerability issues.

As mentioned, for administration to a patient, the chimeric protein effector cells can be allogeneic or autologous to the patient. In certain embodiments, allogeneic cells are further genetically modified, for example by gene editing, so as to minimize or prevent GVHD and/or a patient's immune response against the chimeric protein cells.

The chimeric protein effector cells are used to treat cancers and neoplastic diseases associated with a target antigen. Cancers and neoplastic diseases that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the chimeric protein effector cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 104 to 10⁹ cells/kg body weight, in some instances 105 to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

A chimeric protein-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A chimeric protein-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The chimeric protein therapy and/or other therapeutic agents, procedures or modalities can be administered during periods of active disorder, or during a period of remission or less active disease. The chimeric protein therapy can be administered before the other treatment, concurrently with the treatment, post-treatment, or during remission of the disorder.

When administered in combination, the therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the chimeric protein therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the chimeric protein therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a chimeric protein-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971.

In certain instances, compounds of the present invention are combined with other therapeutic agents, such as other anti-cancer agents, anti-allergic agents, anti-nausea agents (or anti-emetics), pain relievers, cytoprotective agents, and combinations thereof.

In one embodiment, a chimeric protein-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®).

In embodiments, general chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

TCR incorporated antigen agnostic receptors (TIAARs)

Table 1 provides exemplary, non-limiting examples of components of TCR incorporated antigen agnostic receptors (TIAARs) of the invention. Table 2 shows exemplary arrangements of the components.

TABLE 1
TCR incorporated antigen agnostic receptor (TIAAR) components

| | Signal | | | | |
|---|---|---|---|---|---|
| Code | peptide | Tag | ECD_TMD | ICD | Costim |
| pIB1026 | CD3D | Myc | CD3D | N/A | CD28-CD40 |
| pIB1027 | CD3E | FLAG | CD3E | N/A | CD28-CD40 |
| pIB1028 | CD3G | Myc | CD3G | N/A | CD28-CD40 |
| pIB1029 | CD3Z | Myc IC | CD3Z | N/A | CD28-CD40 |
| pIB1030 | CD8A | Myc | hTRDC | N/A | CD28-CD40 |
| pIB1031 | CD8A | FLAG | hTRGC1 | N/A | CD28-CD40 |
| pIB1032 | CD8A | Myc | mTRAC | N/A | CD28-CD40 |
| pIB1033 | CD8A | FLAG | mTRBC1 | N/A | CD28-CD40 |
| pIB1046 | CD8A x2 | Myc and | hTRDC_hTRGC1 | N/A | CD28-CD40 |
| | | FLAG | | | |
| pIB1047 | CD8A x2 | Myc and | mTRAC_mTRBC1 | N/A | CD28-CD40 |
| | | FLAG | | | |
| pIB1048 | CD3D and | Myc and | CD3D_CD3E | N/A | CD28-CD40 |
| | CD3E | FLAG | | | |
| pIB1049 | CD3G and | Myc and | CD3G_CD3E | N/A | CD28-CD40 |
| | CD3E | FLAG | | | |
| pIB1050 | CD3D and | Myc and | CD3D_CD3E | CD3D_CD3E | CD28-CD40 |
| | CD3E | FLAG | | | |
| pIB1051 | CD3D and | Myc and | CD3D_CD3E | CD3D_CD3E | N/A |
| | CD3E | FLAG | | | |
| pIB1052 | CD3D and | Myc and | CD3D_CD3E | N/A | N/A |
| | CD3E | FLAG | | | |
| pIB1053 | CD3G and | Myc and | CD3G_CD3E | CD3G_CD3E | CD28-CD40 |
| | CD3E | FLAG | | | |
| pIB1054 | CD3G and | Myc and | CD3G_CD3E | CD3G_CD3E | N/A |
| | CD3E | FLAG | | | |
| pIB1055 | CD3G and | Myc and | CD3G_CD3E | N/A | N/A |
| | CD3E | FLAG | | | |
| pIB1056 | CD3Z | Myc IC | CD3Z | CD3Z | CD28-CD40 |
| pIB1057 | CD3Z | Myc IC | CD3Z | CD3Z | N/A |
| pIB1058 | CD3Z | Myc IC | CD3Z | N/A | N/A |
| pIB1059 | CD3Z | Myc IC | CD3Z | CD3Z (x2) | CD28-CD40 |
| pIB1060 | CD3Z | Myc IC | CD3Z | CD3Z (x2) | N/A |
| pIB1061 | CD3Z | Myc IC | CD3Z | CD3Z (x2) | CD28-CD40 |
| | | | | | (swaped) |
| pIB1062 | CD3Z | Myc IC | CD3Z | CD3Z | CD28-CD40 |
| | | | | | (swaped) |
| pIB1063 | CD80 | Myc | CD80 | CD80 | N/A |
| pIB1064 | no signal | Myc IC | N/A | Lck | N/A |
| | peptide | | | | |
| pIB1065 | no signal | Myc IC | N/A | Lck (Y505F) | N/A |
| | peptide | | | | |
| pIB1066 | CD80 | Myc | CD80 | Lck | N/A |
| pIB1067 | CD80 | Myc | CD80 | Lck | CD28-CD40 |
| pIB1068 | CD80 | Myc | CD80 | CD80_Lck | N/A |
| | | | | (Y505F) | |
| pIB1069 | CD80 | Myc | CD80 | CD80_Lck | CD28-CD40 |
| | | | | (Y505F) | |
| pIB1070 | CD8A | Myc | LAT | LAT | N/A |
| pIB1071 | CD8A | Myc | LAT | LAT | CD28-CD40 |
| pIB1072 | CD4 | Myc | CD4 | CD4 | CD28-CD40 |
| pIB1073 | CD4 | Myc | CD4 | CD4 | CD28-CD40 |
| pIB1074 | CD8A and | Myc and | CD8A and | CD8A and | N/A |
| | CD8B | FLAG | CD8B | CD8B | |
| pIB1075 | CD8A and | Myc and | CD8A and | CD8A and | CD28-CD40 |
| | CD8B | FLAG | CD8B | CD8B | |

TABLE 2
TCR incorporated antigen agnostic receptors (TIAARs)

| Code | Description |
|---|---|
| pIB1026 | CD3D_CD3D_CD28CD40 |
| pIB1027 | CD3E_CD3E_CD28CD40 |
| pIB1028 | CD3G_CD3G_CD28CD40 |
| pIB1029 | CD3Z_CD3Z_CD28CD40_Myc |
| pIB1030 | CD8A_hTRDC_CD28CD40 |
| pIB1031 | CD8A_hTRGC1_CD28CD40 |
| pIB1032 | CD8A_mTRAC_CD28CD40 |
| pIB1033 | CD8A_mTRBC1_CD28CD40 |
| pIB1046 | CD8A_hTRDC_CD28CD40-T2A-CD8a_hTRGC1_CD28CD40 |
| pIB1047 | CD8A_mTRAC_CD28CD40-T2A-CD8A_mTRBC1_CD28CD40 |
| pIB1048 | CD3D_CD3D_CD28CD40-T2A-CD3E_CD3E_CD28CD40 |
| pIB1049 | CD3G_CD3G_CD28CD40-T2A-CD3E_CD3E_CD28CD40 |
| pIB1050 | CD3D_CD3D_CD3D (ICD)_CD28CD40-T2A-CD3E_CD3E_CD3E |
| | (ICD)_CD28CD40 |
| pIB1051 | CD3D_CD3D_CD3D ICD -T2A-CD3E_CD3E_CD3E ICD |
| pIB1052 | CD3D_CD3D (control)-T2A-CD3E_CD3E (control) |
| pIB1053 | CD3G_CD3G_CD3G (ICD)_CD28CD40-T2A-CD3E_CD3E_CD3E |
| | (ICD)_CD28CD40 |
| pIB1054 | CD3G_CD3G_CD3G ICD -T2A-CD3E_CD3E_CD3E ICD |
| pIB1055 | CD3G_CD3G (control)-T2A-CD3E_CD3E (control) |
| pIB1056 | CD3z_CD3z_CD3z ICD_CD28CD40_Myc |
| pIB1057 | CD3z_CD3z_CD3z ICD_Myc |
| pIB1058 | CD3z_CD3z (control) |
| pIB1059 | CD3Z_CD3Z_CD3Z ICD (duplicating CD3z endodomain-6 |
| | ITAMs)_CD28CD40_Myc |
| pIB1060 | CD3Z_CD3Z_CD3Z ICD (duplicating CD3z endodomain-6 |
| | ITAMs)_Myc |
| pIB1061 | CD3Z_CD3Z_CD28CD40_CD3Z (6 ITAMs)_Myc |
| pIB1062 | CD3Z_CD3Z_CD28CD40_CD3Z ICD_Myc |
| pIB1063 | CD80 (control) |
| pIB1064 | Lck (control) |
| pIB1065 | Lck (Y505F) (control) |
| pIB1066 | CD80_Lck |
| pIB1067 | CD80_Lck_CD28CD40 |
| pIB1068 | CD80_Lck (Y505F) |
| pIB1069 | CD80_Lck (Y505F)_CD28CD40 |
| pIB1070 | LAT (control) |
| pIB1071 | LAT_C28CD40 |
| pIB1072 | CD4 control |
| pIB1073 | CD4_CD28_CD40 |
| pIB1074 | CD8 control |
| pIB1075 | CD8_CD28_CD40 |

Constitutive Costimulatory Proteins

Table 3 provides exemplary, non-limiting examples of components of constitutive costimulatory proteins of the invention. Table 4 shows the exemplary arrangements of the components.

| | Signal | | | |
|---|---|---|---|---|
| Code | peptide | Tag | ECD_TMD | Costim |
| pIB1076 | CD8A | Myc | LZ (cFos)_EGFR | CD28-CD40 |
| pIB1077 | CD8A | Myc | LZ (cFos)_CD28 | CD28-CD40 |
| pIB1078 | CD8A | Myc | LZ (cJun)_EGFR | CD28-CD40 |
| pIB1079 | CD8A | Myc | LZ (cJun)_CD28 | CD28-CD40 |
| pIB1080 | CD8A | Myc | LZ (c/EBP)_EGFR | CD28-CD40 |
| pIB1081 | CD8A | Myc | LZ (c/EBP)_CD28 | CD28-CD40 |
| pIB1103 | GpA | Myc | GpA ECD_TMD | CD28-CD40 |
| pIB1104 | GpA | Myc | GpA TMD | CD28-CD40 |
| pIB1105 | EPOR | Myc | EPOR ECD TMD | CD28-CD40 |
| pIB1106 | EPOR | Myc | EPOR TMD | CD28-CD40 |
| pIB1107 | TPOR | Myc | TPOR ECD_TMD | CD28-CD40 |
| pIB1108 | TPOR | Myc | TPOR TMD | CD28-CD40 |
| pIB1109 | TPOR | Myc | TPOR ECD_TMD (S505N) | CD28-CD40 |
| pIB1110 | TPOR | Myc | TPOR TMD (S505N) | CD28-CD40 |
| pIB1111 | TPOR | Myc | TPOR ECD_TMD (W515K) | CD28-CD40 |
| pIB1112 | TPOR | Myc | TPOR TMD (W515K) | CD28-CD40 |
| pIB1113 | TPOR | Myc | TPOR ECD_TMD (H499L) | CD28-CD40 |
| pIB1114 | TPOR | Myc | TPOR TMD (H499L) | CD28-CD40 |
| pIB1115 | TPOR | Myc | TPOR ECD_TMD (S505N- | CD28-CD40 |
| | | | W515K) | |
| pIB1116 | TPOR | Myc | TPOR TMD (S505N-W515K) | CD28-CD40 |
| pIB1117 | TPOR | Myc | TPOR ECD_TMD (H499Y- | CD28-CD40 |
| | | | S505N) | |
| pIB1118 | TPOR | Myc | TPOR TMD (H499Y-S505N) | CD28-CD40 |
| pIB1119 | TPOR | Myc | TPOR ECD_TMD (L498W- | CD28-CD40 |
| | | | H499C) | |
| pIB1120 | TPOR | Myc | TPOR TMD (L498W-H499C) | CD28-CD40 |
| pIB1025 | CD8a | Myc | CD28 | CD28-CD40 |
| pIB1179 | CD8a | N/A | IgG1 + CD28TM | CD28-CD40 |
| pIB1180 | CD8a | N/A | IgG1mut + CD28TM | CD28-CD40 |
| pIB1181 | CD8a | N/A | IgG2 + CD28TM | CD28-CD40 |
| pIB1182 | CD8a | N/A | IgG3 + CD28TM | CD28-CD40 |
| pIB1183 | CD8a | N/A | IgG4 + CD28TM | CD28-CD40 |
| pIB1184 | CD8a | N/A | IgG4mut + CD28TM | CD28-CD40 |
| pIB1185 | CD8a | N/A | IgG1 + CD28 stalk/TM | CD28-CD40 |
| pIB1186 | CD8a | N/A | IgG1mut + CD28 stalk/TM | CD28-CD40 |
| pIB1187 | CD8a | N/A | IgG2 + CD28 stalk/TM | CD28-CD40 |
| pIB1188 | CD8a | N/A | IgG3 + CD28 stalk/TM | CD28-CD40 |
| pIB1189 | CD8a | N/A | IgG4 + CD28 stalk/TM | CD28-CD40 |
| pIB1190 | CD8a | N/A | IgG4mut + CD28 stalk/TM | CD28-CD40 |

TABLE 4
C-SAAR Proteins

| Code | Description |
|---|---|
| pIB1076 | LZ (cFos)- EGFRTM/JMD- CD28-CD40 |
| pIB1077 | LZ (cFos)- CD28TM- CD28-CD40 |
| pIB1078 | LZ (cJun)- EGFRTM/JMD- CD28-CD40 |
| pIB1079 | LZ (cJun)- CD28TM- CD28-CD40 |
| pIB1080 | LZ (c/EBP)- EGFRTM/JMD- CD28-CD40 |
| pIB1081 | LZ (c/EBP)- CD28TM- CD28-CD40 |
| pIB1103 | GpA ECD-TMD-CD28-CD40 |
| pIB1104 | GpA TMD-CD28-CD40 |
| pIB1105 | EpoR ECD-TMD-CD28-CD40 |
| pIB1106 | EpoR TMD-CD28-CD40 |
| pIB1107 | TPO ECD- TPO (WT) TMD -CD28-CD40 |
| pIB1108 | TPO (WT) TMD -CD28-CD40 |
| pIB1109 | TPO ECD- TPO (S505N) TMD -CD28-CD40 |
| pIB1110 | TPO (S505N) TMD -CD28-CD40 |
| pIB1111 | TPO ECD- TPO (W515K) TMD -CD28-CD40 |
| pIB1112 | TPO (W515K) TMD -CD28-CD40 |
| pIB1113 | TPO ECD- TPO (H499L) TMD -CD28-CD40 |
| pIB1114 | TPO (H499L) TMD -CD28-CD40 |
| pIB1115 | TPO ECD- TPO (S505N-W515K) TMD -CD28-CD40 |
| pIB1116 | TPO (S505N-W515K) TMD -CD28-CD40 |
| pIB1117 | TPO ECD- TPO (H499Y-S505N) TMD -CD28-CD40 |
| pIB1118 | TPO (H499Y-S505N) TMD -CD28-CD40 |
| pIB1119 | TPO ECD- TPO (L498W-H499C) TMD -CD28-CD40 |
| pIB1120 | TPO (L498W-H499C) TMD -CD28-CD40 |
| pIB1025 | CD28 TM_CD28_CD40 |
| pIB1179 | IgG1(CH2CH3)-CD28(TM)-CD28(CoStim)-CD40(CoStim) |
| pIB1180 | IgG1(CH2CH3, mutant)-CD28(TM)-CD28(CoStim)-CD40(CoStim) |
| pIB1181 | IgG2(CH2CH3)-CD28(TM)-CD28(CoStim)-CD40(CoStim) |
| pIB1182 | IgG3(CH2CH3)-CD28(TM)-CD28(CoStim)-CD40(CoStim) |
| pIB1183 | IgG4(CH2CH3)-CD28(TM)-CD28(CoStim)-CD40(CoStim) |
| pIB1184 | IgG4(CH2CH3, mutant)-CD28(TM)-CD28(CoStim)-CD40(CoStim) |
| pIB1185 | IgG1(CH2CH3)-CD28(Stalk + TM)-CD28(CoStim)-CD40(CoStim) |
| pIB1186 | IgG1(CH2CH3, mutant)-CD28(Stalk + TM)-CD28(CoStim)-CD40(CoStim) |
| pIB1187 | IgG2(CH2CH3)-CD28(Stalk + TM)-CD28(CoStim)-CD40(CoStim) |
| pIB1188 | IgG3(CH2CH3)-CD28(Stalk + TM)-CD28(CoStim)-CD40(CoStim) |
| pIB1189 | IgG4(CH2CH3)-CD28(Stalk + TM)-CD28(CoStim)-CD40(CoStim) |
| pIB1190 | IgG4(CH2CH3, mutant)-CD28(Stalk + TM)-CD28(CoStim)-CD40(CoStim) |

Inducible Costimulatory Receptors

Table 5 provides exemplary, non-limiting examples of inducible costimulatory receptors of the invention. Table 6 shows exemplary arrangements of the components.

TABLE 5
Inducible costimulatory protein components

| | Signal | | | | |
|---|---|---|---|---|---|
| Code | peptide | Tag | ECD_TMD | ICD | Costim |
| pIB1082 | EGFR | Myc | EGFR | N/A | CD28-CD40 |
| pIB1083 | EGFR | Myc | EGFR (domain IV) | N/A | CD28-CD40 |
| pIB1084 | EGFR | Myc | EGFR (623-668) | N/A | CD28-CD40 |
| pIB1085 | Her2 | Myc | Her2 | N/A | CD28-CD40 |
| pIB1086 | Her2 | Myc | Her2 (V659E) | N/A | CD28-CD40 |
| pIB1087 | Her2 | Myc | Her2 (V660D) | N/A | CD28-CD40 |
| pIB1088 | Her2 | Myc | Her2 (V660R) | N/A | CD28-CD40 |
| pIB1089 | Her2 | Myc | Her2 domain IV_TMD | N/A | CD28-CD40 |
| pIB1090 | Her2 | Myc | Her2 domain IV_TMD (V659E) | N/A | CD28-CD40 |
| pIB1091 | Her2 | Myc | Her2 domain IV_TMD (G660D) | N/A | CD28-CD40 |
| pIB1092 | Her2 | Myc | Her2 domain IV_TMD (G660R) | N/A | CD28-CD40 |
| pIB1093 | Her2 | Myc | Her2 TMD | N/A | CD28-CD40 |
| pIB1094 | Her2 | Myc | Her2 TMD (V659E) | N/A | CD28-CD40 |
| pIB1095 | Her2 | Myc | Her2 TMD (G660D) | N/A | CD28-CD40 |
| pIB1096 | Her2 | Myc | Her2 TMD (G660R) | N/A | CD28-CD40 |
| pIB1097 | CD8A | Myc | A30514 VH_VL | N/A | CD28-CD40 |
| pIB1098 | CD8A | Myc | A30514 VL_VH | N/A | CD28-CD40 |
| pIB1099 | CD8A | Myc | A30523 VH_VL | N/A | CD28-CD40 |
| pIB1100 | CD8A | Myc | A30523 VL_VH | N/A | CD28-CD40 |
| pIB1101 | CD8A | Myc | A30633 VH_VL | N/A | CD28-CD40 |
| pIB1102 | CD8A | Myc | A30633 VL_VH | N/A | CD28-CD40 |

TABLE 6
Inducible costimulatory proteins

| Code | GOI description |
|---|---|
| pIB1082 | WT EGFR ECD- EGFRTM/JMD- CD28-CD40 |
| pIB1083 | domain IV - EGFRTM/JMD- CD28-CD40 |
| pIB1084 | EGFRTM/JMD- CD28-CD40 (control) |
| pIB1085 | Her 2 (Domain 1 to IV)-TMD/JMD-CD28-CD40 |
| pIB1086 | Her 2 (Domain 1 to IV)-TMD (V659E)/JMD-CD28-CD40 |
| pIB1087 | Her 2 (Domain 1 to IV)-TMD (G660D)/JMD-CD28-CD40 |
| pIB1088 | Her 2 (Domain 1 to IV)-TMD (G660R)/JMD-CD28-CD40 |
| pIB1089 | Her 2 (Domain IV)-TMD/JMD-CD28-CD40 |
| pIB1090 | Her 2 (Domain IV)-TMD (V659E)/JMD-CD28-CD40 |
| pIB1091 | Her 2 (Domain IV)-TMD (G660D)/JMD-CD28-CD40 |
| pIB1092 | Her 2 (Domain IV)-TMD (G660R)/JMD-CD28-CD40 |
| pIB1093 | Her 2 TMD/JMD-CD28-CD40 |
| pIB1094 | Her 2 TMD (V659E)/JMD-CD28-CD40 |
| pIB1095 | Her 2 TMD (G660D)/JMD-CD28-CD40 |
| pIB1096 | Her 2 TMD (G660R)/JMD-CD28-CD40 |
| pIB1097 | Anti-ID1 VH-VL (A30514-pembro)- CD28TMD CD28-CD40 |
| pIB1098 | Anti-ID1 VL-VH (A30514-pembro)- CD28TMD CD28-CD40 |
| pIB1099 | Anti-ID2 Vh-VL (A30523-pembro)- CD28TMD CD28-CD40 |
| pIB1100 | Anti-ID2 VL-Vh (A30523-pembro)- CD28TMD CD28-CD40 |
| pIB1101 | Anti-ID3 Vh-VL (A30633-pembro)- CD28TMD CD28-CD40 |
| pIB1102 | Anti-ID3 VL-VH (A30633-pembro)- CD28TMD CD28-CD40 |

The following sequences in the below table include complete components and are non-limiting. Components may include a signal peptide (SP), a TCR clustering domain (CD) and/or a signaling domain (SD). It will be understood that whereas certain proteins may comprise N-terminal signal peptides when expressed, those signal peptides are cleaved and may be imprecisely cleaved when the proteins are expressed, and that the resulting proteins from which signal peptides are removed comprise binding domains having variation of up to about five amino acids in the location of the N-terminal amino acid.

TABLE
TCR costimulation construct examples

| | |
|---|---|
| SEQ ID NO: 1 | |
| Component: | SP CD3D_ |
| Sequence: | MEHSTFLSGL VLATLLSQVS P |
| SEQ ID NO: 2 | |
| Component: | CD CD3D_ |
| Sequence: | FKIPIEELED RVFVNCNTSI TWVEGTVGTL LSDITRLDLG KRILDPRGIY |
| | RCNGTDIYKD KESTVQVHYR MCQSCVELDP ATVAGIIVTD VIATLLLALG |
| | VFCFA |
| SEQ ID NO: 3 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 4 | |
| Full length: | CD3D_CD3D_CD28CD40 |
| Sequence: | MEHSTFLSGL VLATLLSQVS PFKIPIEELE DRVFVNCNTS ITWVEGTVGT |
| | LLSDITRLDL GKRILDPRGI YRCNGTDIYK DKESTVQVHY RMCQSCVELD |
| | PATVAGIIVT DVIATLLLAL GVFCFARSKR SRLLHSDYMN MTPRRPGPTR |
| | KHYQPYAPPR DFAAYRSKKV AKKPTNKAPH PKQEPQEINF PDDLPGSNTA |
| | APVQETLHGC QPVTQEDGKE SRISVQERQ |
| SEQ ID NO: 5 | |
| Component: | SP CD3E_ |
| Sequence: | MQSGTHWRVL GLCLLSVGVW GQ |
| SEQ ID NO: 6 | |
| Component: | CD CD3E_ |
| Sequence: | DGNEEMGGIT QTPYKVSISG TTVILTCPQY PGSEILWOHN DKNIGGDEDD |
| | KNIGSDEDHL SLKEFSELEQ SGYYVCYPRG SKPEDANFYL YLRARVCENC |
| | MEMDVMSVAT IVIVDICITG GLLLLVYYWS |
| SEQ ID NO: 7 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 8 | |
| Full length: | CD3E_CD3E_CD28CD40 |
| Sequence: | MQSGTHWRVL GLCLLSVGVW GQDGNEEMGG ITQTPYKVSI |
| | SGTTVILTCPQ |
| | YPGSEILWQH NDKNIGGDED DKNIGSDEDH LSLKEFSELE |
| | QSGYYVCYPRG |
| | SKPEDANFYL YLRARVCENC MEMDVMSVAT IVIVDICITG |
| | GLLLLVYYWSR |
| | SKRSRLLHSD YMNMTPRRPG PTRKHYQPYA PPRDFAAYRS |
| | KKVAKKPTNKA |
| | PHPKQEPQEI NFPDDLPGSN TAAPVQETLH GCQPVTQEDG |
| | KESRISVQERQ |
| SEQ ID NO: 9 | |
| Component: | SP CD3G_ |
| Sequence: | MEQGKGLAVL ILAIILLQGT LA |
| SEQ ID NO: 10 | |
| Component: | CD CD3G_ |
| Sequence: | QSIKGNHLVK VYDYQEDGSV LLTCDAEAKN ITWFKDGKMI GFLTEDKKKW |
| | NLGSNAKDPR GMYQCKGSQN KSKPLQVYYR MCQNCIELNA ATISGFLFAE |
| | IVSIFVLAVG VYFIA |
| SEQ ID NO: 11 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 12 | |
| Full length: | CD3G_CD3G_CD28CD40 |
| Sequence: | MEQGKGLAVL ILAIILLQGT LAQSIKGNHL VKVYDYQEDG SVLLTCDAEA |
| | KNITWFKDGK MIGFLTEDKK KWNLGSNAKD PRGMYQCKGS QNKSKPLQVY |
| | YRMCQNCIEL NAATISGFLF AEIVSIFVLA VGVYFIARSK RSRLLHSDYM |
| | NMTPRRPGPT RKHYQPYAPP RDFAAYRSKK VAKKPTNKAP HPKQEPQEIN |
| | FPDDLPGSNT AAPVQETLHG CQPVTQEDGK ESRISVQERQ |
| SEQ ID NO: 13 | |
| Component: | SP CD3Z_ |
| Sequence: | MKWKALFTAA ILQAQLPITE A |
| SEQ ID NO: 14 | |
| Component: | CD CD3Z_ |
| Sequence: | QSFGLLDPKL CYLLDGILFI YGVILTALFL |
| SEQ ID NO: 15 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 16 | |
| Full length: | CD3Z_CD3Z_CD28CD40 |
| Sequence: | MKWKALFTAA ILQAQLPITE AQSFGLLDPK LCYLLDGILF IYGVILTALF |
| | LRSKRSRLLH SDYMNMTPRR PGPTRKHYQP YAPPRDFAAY RSKKVAKKPT |
| | NKAPHPKQEP QEINFPDDLP GSNTAAPVQE TLHGCQPVTQ EDGKESRISV |
| | QERQ |
| SEQ ID NO: 17 | |
| Component: | SP CD8A_ |
| Sequence: | MALPVTALLL PLALLLHAAR P |
| SEQ ID NO: 18 | |
| Component: | CD TRDC_ |
| Sequence: | SQPHTKPSVF VMKNGTNVAC LVKEFYPKDI RINLVSSKKI TEFDPAIVIS |
| | PSGKYNAVKL GKYEDSNSVT CSVQHDNKTV HSTDFEVKTD STDHVKPKET |
| | ENTKQPSKSC HKPKAIVHTE KVNMMSLTVL GLRMLFAKTV AVNFLLTAKL |
| | FFL |
| SEQ ID NO: 19 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 20 | |
| Full length: | CD8A_TRDC_CD28CD40 |
| Sequence: | MALPVTALLL PLALLLHAAR PSQPHTKPSV FVMKNGTNVA CLVKEFYPKD |
| | IRINLVSSKK ITEFDPAIVI SPSGKYNAVK LGKYEDSNSV TCSVQHDNKT |
| | VHSTDFEVKT DSTDHVKPKE TENTKQPSKS CHKPKAIVHT EKVNMMSLTV |
| | LGLRMLFAKT VAVNFLLTAK LFFLRSKRSR LLHSDYMNMT PRRPGPTRKH |
| | YQPYAPPRDF AAYRSKKVAK KPTNKAPHPK QEPQEINFPD DLPGSNTAAP |
| | VQETLHGCQP VTQEDGKESR ISVQERQ |
| SEQ ID NO: 21 | |
| Component: | SP CD8A_ |
| Sequence: | MALPVTALLL PLALLLHAAR P |
| SEQ ID NO: 22 | |
| Component: | CD TRGC1_ |
| Sequence: | DKQLDADVSP KPTIFLPSIA ETKLQKAGTY LCLLEKFFPD VIKIHWQEKK |
| | SNTILGSQEG NTMKTNDTYM KESWLTVPEK SLDKEHRCIV RHENNKNGVD |
| | QEIIFPPIKT DVITMDPKDN CSKDANDTLL LQLTNTSAYY MYLLLLLKSV |
| | VYFAIITCCL L |
| SEQ ID NO: 23 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 24 | |
| Full length: | CD8A_TRGC1_CD28CD40 |
| Sequence: | MALPVTALLL PLALLLHAAR PDKQLDADVS PKPTIFLPSI AETKLQKAGT |
| | YLCLLEKFFP DVIKIHWQEK KSNTILGSQE GNTMKTNDTY MKFSWLTVPE |
| | KSLDKEHRCI VRHENNKNGV DQEIIFPPIK TDVITMDPKD NCSKDANDTL |
| | LLQLTNTSAY YMYLLLLLKS VVYFAIITCC LLRSKRSRLL HSDYMNMTPR |
| | RPGPTRKHYQ PYAPPRDFAA YRSKKVAKKP TNKAPHPKQE PQEINFPDDL |
| | PGSNTAAPVQ ETLHGCQPVT QEDGKESRIS VQERQ |
| SEQ ID NO: 25 | |
| Component: | SP CD8A_ |
| Sequence: | MALPVTALLL PLALLLHAAR P |
| SEQ ID NO: 26 | |
| Component: | CD TRAC_ |
| Sequence: | IQNPEPAVYQ LKDPRSQDST LCLFTDFDSQ INVPKTMESG TFITDKCVLD |
| | MKAMDSKSNG AIAWSNQTSF TCQDIFKETN ATYPSSDVPC DATLTEKSFE |
| | TDMNLNFQNL LVIVLRILLL KVAGFNLLMT LRLWSS |
| SEQ ID NO: 27 | |
| Component: | SD CD28CD40 |
| Sequence: | RSKRSRLLHS DYMNMTPRRP GPTRKHYQPY APPRDFAAYR SKKVAKKPTN |
| | KAPH PKQEP QEINFPDDLP GSNTAAPVQE TLHGCQPVTQ EDGKESRISV |
| | QERQ |
| SEQ ID NO: 28 | |
| Full length: | CD8A_TRAC_CD28CD40 |
| Sequence: | MALPVTALLL PLALLLHAAR PIQNPEPAVY QLKDPRSQDS TLCLFTDFDS |
| | QINVPKTMES GTFITDKCVL DMKAMDSKSN GAIAWSNQTS FTCQDIFKET |
| | NATYPSSDVP CDATLTEKSF ETDMNLNFQN LLVIVLRILL LKVAGENLLM |
| | TLRLWSSRSK RSRLLHSDYM NMTPRRPGPT RKHYQPYAPP RDFAAYRSKK |
| | VAKKPTNKAP HPKQEPQEIN FPDDLPGSNT AAPVQETLHG CQPVTQEDGK |
| | ESRISVQERQ |
| SEQ ID NO: 29 | |
| Component: | SP CD8A_ |
| Sequence: | MALPVTALLL PLALLLHAAR P |
| SEQ ID NO: 30 | |
| Component: | CD TRBC1_ |
| Sequence: | VLTPPKVSLF EPSKAEIANK QKATLVCLAR GFFPDHVELS WWVNGKEVHS |
| | GVCTDPQAYK ESNYSYCLSS RLRVSATFWH NPRNHFRCQV QFHGLSEEDK |
| | WPEGSPKPVT QNISAEAWGR ADCGITSASY QQGVLSATIL YEILLGKATL |
| | YAVLVSTLVV MAMVKRKNS |
| SEQ ID NO: 31 | |
| Component: | SD CD28CD40 |
| Sequence: | RSKRSRLLHS DYMNMTPRRP GPTRKHYQPY APPRDFAAYR SKKVAKKPTN |
| | KAPHPKQEPQ EINFPDDLPG SNTAAPVQET LHGCQPVTQE DGKESRISVQ |
| | ERQ |
| SEQ ID NO: 32 | |
| Full length: | CD8A_TRBC1_CD28CD40 |
| Sequence: | MALPVTALLL PLALLLHAAR PVLTPPKVSL FEPSKAEIAN KQKATLVCLA |
| | RGFFPDHVEL SWWVNGKEVH SGVCTDPQAY KESNYSYCLS SRLRVSATFW |
| | HNPRNHFRCQ VQFHGLSEED KWPEGSPKPV TQNISAEAWG RADCGITSAS |
| | YQQGVLSATI LYEILLGKAT LYAVLVSTLV VMAMVKRKNS RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 33 | |
| vector clone: | pIB1001 |
| Sequence | MALPVTALLL PLALLLHAAR PEQKLISEED LQVQLVQSGA EVKKPGASVK |
| | VSCKASGYTF TSYWMNWVRQ APGQGLEWMG RIDPYDSETH YAQKLQGRVT |
| | MTTDTSTSTA YMELRSLRSD DTAVYYCARG GYDFDVGTLY WFFDVWGQGT |
| | TVTVSSGGGG SGGGGSGGGG SDIQMTQSPS SLSASVGDRV TITCRASENI |
| | YSYLAWYQQK PGKAPKLLIY NAKTLAEGVP SRESGSGSGT DFTLTISSLQ |
| | PEDFATYYCQ HHYGTPRTFG GGTKVEIKAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 34 | |
| vector clone: | pIB1002 |
| Sequence: | MALPVTALLL PLALLLHAAR PEQKLISEED LDIQMTQSPS SLSASVGDRV |
| | TITCRASENI YSYLAWYQQK PGKAPKLLIY NAKTLAEGVP SRFSGSGSGT |
| | DFTLTISSLQ PEDFATYYCQ HHYGTPRTFG GGTKVEIKGG GGSGGGGSGG |
| | GGSQVQLVQS GAEVKKPGAS VKVSCKASGY TFTSYWMNWV RQAPGQGLEW |
| | RGGYDFDVGT LYWFFDVWGQ VTMTTDTSTS TAYMELRSLR SDDTAVYYCA |
| | MGRIDPYDSE THYAQKLQGR GTTVTVSSAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 35 | |
| vector clone: | pIB1003 |
| Sequence: | MALPVTALLL PLALLLHAAR PEQKLISEED LQVQLVESGG GVVQPGRSLR |
| | LSCAASGFTF SSYDMHWVRQ APGKGLEWVA VIWYDGSNKY YADSVKGRFT |
| | ISRDNSKNTL YLQMNSLRAE DTAVYYCARG SGNWGFFDYW GQGTLVTVSS |
| | GGGGSGGGGS GGGGSDIQMT QSPSSLSASV GDRVTITCRA SQGISRWLAW |
| | YQQKPEKAPK SLIYAASSLQ SGVPSRFSGS GSGTDFTLTI SSLQPEDFAT |
| | YYCQQYNTYP RTFGQGTKVE IKAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 36 | |
| vector clone: | pIB1004 |
| Sequence: | MALPVTALLL PLALLLHAAR PEQKLISEED LDIQMTQSPS SLSASVGDRV |
| | TITCRASQGI SRWLAWYQQK PEKAPKSLIY AASSLQSGVP SRFSGSGSGT |
| | DFTLTISSLQ PEDFATYYCQ QYNTYPRTFG QGTKVEIKGG GGSGGGGSGG |
| | GGSQVQLVES GGGVVQPGRS LRLSCAASGF TFSSYDMHWV RQAPGKGLEW |
| | VAVIWYDGSN KYYADSVKGR FTISRDNSKN TLYLQMNSLR AEDTAVYYCA |
| | RGSGNWGFFD YWGQGTLVTV SSAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 37 | |
| vector clone: | pIB1005 |
| Sequence: | MALPVTALLL PLALLLHAAR PEQKLISEED LQVQLQQWGA GLLKPSETLS |
| | LTCAVYGGSF SGYYWSWIRQ SPEGLEWIGE INHGGYVTYN PSLESRVTIS |
| | VDTSKNQFSL KLSSVTAADT AVYYCARDYG PGNYDWYFDL WGRGTLVTVS |
| | SGGGGSGGGG SGGGGSEIVL TQSPATLSLS PGERATLSCR ASQSVSSYLA |
| | WYQQKPGQAP RLLIYDASNR ATGIPARFSG SGSGTDFTLT ISSLEPEDFA |
| | VYYCQQRSNW PPALTFGGGT KVEIKRAAAG SGGSGILVKQ SPMLVAYDNA |
| | VNLSCKYSYN LESREFRASL HKGLDSAVEV CVVYGNYSQQ LQVYSKTGFN |
| | CDGKLGNESV TFYLQNLYVN QTDIYFCKIE VMYPPPYLDN EKSNGTIIHV |
| | KGKHLCPSPL FPGPSKPFWV LVVVGGVLAC YSLLVTVAFI IFWVRSKRSR |
| | LLHSDYMNMT PRRPGPTRKH YQPYAPPRDF AAYRSKKVAK KPTNKAPHPK |
| | QEPQEINFPD DLPGSNTAAP VQETLHGCQP VTQEDGKESR ISVQERQ |
| SEQ ID NO: 38 | |
| vector clone : | pIB1006 |
| Sequence: | MALPVTALLL PLALLLHAAR PEQKLISEED LEIVLTQSPA TLSLSPGERA |
| | TLSCRASQSV SSYLAWYQQK PGQAPRLLIY DASNRATGIP ARFSGSGSGT |
| | DFTLTISSLE PEDFAVYYCQ QRSNWPPALT FGGGTKVEIK RGGGGSGGGG |
| | SGGGGSQVQL QQWGAGLLKP SETLSLTCAV YGGSFSGYYW SWIRQSPEGL |
| | EWIGEINHGG YVTYNPSLES RVTISVDTSK NQFSLKLSSV TAADTAVYYC |
| | ARDYGPGNYD WYFDLWGRGT LVTVSSAAAG SGGSGILVKQ SPMLVAYDNA |
| | VNLSCKYSYN LFSREFRASL HKGLDSAVEV CVVYGNYSQQ LQVYSKTGFN |
| | CDGKLGNESV TFYLQNLYVN QTDIYFCKIE VMYPPPYLDN EKSNGTIIHV |
| | KGKHLCPSPL FPGPSKPFWV LVVVGGVLAC YSLLVTVAFI IFWVRSKRSR |
| | LLHSDYMNMT PRRPGPTRKH YQPYAPPRDF AAYRSKKVAK KPTNKAPHPK |
| | QEPQEINFPD DLPGSNTAAP VQETLHGCQP VTQEDGKESR ISVQERQ |
| SEQ ID NO: 39 | |
| vector clone: | pIB1007 |
| Sequence: | MALPVTALLL PLALLLHAAR PEQKLISEED LQVTLRESGP ALVKPTQTLT |
| | LTCTFSGFSL STSGMGVGWI RQPPGKALEW LAHIWWDDDK YYNPSLKSRL |
| | TISKDTSKNQ VVLTMTNMDP VDTATYYCAR TRRYFPFAYW GQGTLVTVSS |
| | GGGGSGGGGS GGGGSEIVMT QSPATLSVSP GERATLSCKA SQNVGTNVAW |
| | YQQKPGQAPR LLIYSASYRY SGIPARFSGS GSGTEFTLTI SSLQSEDFAV |
| | YYCQQYNTDP LTFGGGTKVE IKAAAGSGGS GILVKQSPML VAYDNAVNLS |
| | CKYSYNLESR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 40 | |
| vector clone: | pIB1008 |
| Sequence: | MALPVTALLL PLALLLHAAR PEQKLISEED LEIVMTQSPA TLSVSPGERA |
| | TLSCKASQNV GTNVAWYQQK PGQAPRLLIY SASYRYSGIP ARFSGSGSGT |
| | EFTLTISSLQ SEDFAVYYCQ QYNTDPLTFG GGTKVEIKGG GGSGGGGSGG |
| | GGSQVTLRES GPALVKPTQT LTLTCTFSGF SLSTSGMGVG WIRQPPGKAL |
| | EWLAHIWWDD DKYYNPSLKS RLTISKDTSK NQVVLTMTNM DPVDTATYYC |
| | ARTRRYFPFA YWGQGTLVTV SSAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 41 | |
| vector clone: | pIB1009 |
| Sequence: | MALPVTALLL PLALLLHAAR PEQKLISEED LQVQLVQSGV EVKKPGASVK |
| | VSCKASGYTF TNYYMYWVRQ APGQGLEWMG GINPSNGGTN FNEKFKNRVT |
| | LTTDSSTTTA YMELKSLQFD DTAVYYCARR DYRFDMGFDY WGQGTTVTVS |
| | SGGGGSGGGG SGGGGSEIVL TQSPATLSLS PGERATLSCR ASKGVSTSGY |
| | SYLHWYQQKP GQAPRLLIYL ASYLESGVPA RFSGSGSGTD FTLTISSLEP |
| | EDFAVYYCQH SRDLPLTFGG GTKVEIKRAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 42 | |
| vector clone: | pIB1010 |
| Sequence: | MALPVTALLL PLALLLHAAR PEQKLISEED LEIVLTQSPA TLSLSPGERA |
| | TLSCRASKGV STSGYSYLHW YQQKPGQAPR LLIYLASYLE SGVPARFSGS |
| | GSGTDFTLTI SSLEPEDFAV YYCQHSRDLP LTFGGGTKVE IKRGGGGSGG |
| | GGSGGGGSQV QLVQSGVEVK KPGASVKVSC KASGYTFTNY YMYWVRQAPG |
| | QGLEWMGGIN PSNGGTNFNE KFKNRVTLTT DSSTTTAYME LKSLQFDDTA |
| | VYYCARRDYR FDMGFDYWGQ GTTVTVSSAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 43 | |
| vector clone: | pIB1011 |
| Sequence: | MALPVTALLL PLALLLHAAR PEQKLISEED LEVQLVESGG GLVQPGGSLR |
| | LSCAASGFTF SDSWIHWVRQ APGKGLEWVA WISPYGGSTY YADSVKGRFT |
| | ISADTSKNTA YLQMNSLRAE DTAVYYCARR HWPGGFDYWG QGTLVTVSSG |
| | GGGSGGGGSG GGGSDIQMTQ SPSSLSASVG DRVTITCRAS QDVSTAVAWY |
| | QQKPGKAPKL LIYSASFLYS GVPSRFSGSG SGTDFTLTIS SLQPEDFATY |
| | YCQQYLYHPA TFGQGTKVEI KRAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 44 | |
| vector clone: | pIB1012 |
| Sequence: | MALPVTALLL PLALLLHAAR PEQKLISEED LDIQMTQSPS SLSASVGDRV |
| | TITCRASQDV STAVAWYQQK PGKAPKLLIY SASFLYSGVP SRFSGSGSGT |
| | DFTLTISSLQ PEDFATYYCQ QYLYHPATFG QGTKVEIKRG GGGSGGGGSG |
| | GGGSEVQLVE SGGGLVQPGG SLRLSCAASG FTFSDSWIHW VRQAPGKGLE |
| | WVAWISPYGG STYYADSVKG RFTISADTSK NTAYLQMNSL RAEDTAVYYC |
| | ARRHWPGGFD YWGQGTLVTV SSAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 45 | |
| vector clone: | pIB1013 |
| Sequence: | MALPVTALLL PLALLLHAAR PEQKLISEED LQVQLVQSGA EVKKPGASVK |
| | VSCKASGYTF TNYWIGWVKQ APGQGLEWIG YLYPGGLYTN YNEKFKGKAT |
| | MTADTSTNTA YMELSSLRSE DTAVYYCARY RDYDYAMDYW GQGTLVTVSS |
| | GGGGSGGGGS GGGGSDVVMT QTPLSLPVTL GQPASISCKS TKSLLNSDGF |
| | TYLGWCLQKP GQSPQLLIYL VSNRFSGVPD RFSGSGSGTD FTLKISRVEA |
| | EDVGVYYCFQ SNYLPLTFGQ GTKLEIKRAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 46 | |
| vector clone: | pIB1014 |
| Sequence: | MALPVTALLL PLALLLHAAR PEQKLISEED LDVVMTQTPL SLPVTLGQPA |
| | SISCKSTKSL LNSDGFTYLG WCLQKPGQSP QLLIYLVSNR FSGVPDRFSG |
| | SGSGTDFTLK ISRVEAEDVG VYYCFQSNYL PLTFGQGTKL EIKRGGGGSG |
| | GGGSGGGGSQ VQLVQSGAEV KKPGASVKVS CKASGYTFTN YWIGWVKQAP |
| | GQGLEWIGYL YPGGLYTNYN EKFKGKATMT ADTSTNTAYM ELSSLRSEDT |
| | AVYYCARYRD YDYAMDYWGQ GTLVTVSSAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 47 | |
| vector clone: | pIB1015 |
| Sequence: | MALPVTALLL PLALLLHAAR PEQKLISEED LQVQLQESGP GLVKPSQTLS |
| | LTCAVYGGSF SSGYWNWIRK HPGKGLEYIG YISYNGITYH NPSLKSRITI |
| | NRDTSKNQYS LQLNSVTPED TAVYYCARYK YDYDGGHAMD YWGQGTLVTV |
| | SSGGGGSGGG GSGGGGSDIQ MTQSPSSLSA SVGDRVTITC RASQDISNYL |
| | NWYQQKPGKA PKLLIYYTSK LHSGVPSRFS GSGSGTDYTL TISSLQPEDF |
| | ATYYCQQGSA LPWTFGQGTK VEIKAAAGSG GSGILVKQSP MLVAYDNAVN |
| | LSCKYSYNLF SREFRASLHK GLDSAVEVCV VYGNYSQQLQ VYSKTGFNCD |
| | GKLGNESVTF YLQNLYVNQT DIYFCKIEVM YPPPYLDNEK SNGTIIHVKG |
| | KHLCPSPLFP GPSKPFWVLV VVGGVLACYS LLVTVAFIIF WVRSKRSRLL |
| | HSDYMNMTPR RPGPTRKHYQ PYAPPRDFAA YRSKKVAKKP TNKAPHPKQE |
| | PQEINFPDDL PGSNTAAPVQ ETLHGCQPVT QEDGKESRIS VQERQ |
| SEQ ID NO: 48 | |
| vector clone: | pIB1016 |
| Sequence: | MALPVTALLL PLALLLHAAR PEQKLISEED LDIQMTQSPS SLSASVGDRV |
| | TITCRASQDI SNYLNWYQQK PGKAPKLLIY YTSKLHSGVP SRFSGSGSGT |
| | DYTLTISSLQ PEDFATYYCQ QGSALPWTFG QGTKVEIKGG GGSGGGGSGG |
| | GGSQVQLQES GPGLVKPSQT LSLTCAVYGG SFSSGYWNWI RKHPGKGLEY |
| | IGYISYNGIT YHNPSLKSRI TINRDTSKNQ YSLQLNSVTP EDTAVYYCAR |
| | YKYDYDGGHA MDYWGQGTLV TVSSAAAGSG GSGILVKQSP MLVAYDNAVN |
| | LSCKYSYNLF SREFRASLHK GLDSAVEVCV VYGNYSQQLQ VYSKTGFNCD |
| | GKLGNESVTF YLQNLYVNQT DIYFCKIEVM YPPPYLDNEK SNGTIIHVKG |
| | KHLCPSPLFP GPSKPFWVLV VVGGVLACYS LLVTVAFIIF WVRSKRSRLL |
| | HSDYMNMTPR RPGPTRKHYQ PYAPPRDFAA YRSKKVAKKP TNKAPHPKQE |
| | PQEINFPDDL PGSNTAAPVQ ETLHGCQPVT QEDGKESRIS VQERQ |
| SEQ ID NO: 49 | |
| vector clone: | pIB1017 |
| Sequence: | MALPVTALLL PLALLLHAAR PEQKLISEED LQVQLVESGG GVVQPGRSLR |
| | LSCAASGFTF SSYTMHWVRQ APGKGLEWVT FISYDGNNKY YADSVKGRFT |
| | ISRDNSKNTL YLQMNSLRAE DTAIYYCART GWLGPFDYWG QGTLVTVSSG |
| | GGGSGGGGSG GGGSEIVLTQ SPGTLSLSPG ERATLSCRAS QSVGSSYLAW |
| | YQQKPGQAPR LLIYGAFSRA TGIPDRFSGS GSGTDFTLTI SRLEPEDFAV |
| | YYCQQYGSSP WTFGQGTKVE IKAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 50 | |
| vector clone: | pIB1018 |
| Sequence: | MALPVTALLL PLALLLHAAR PEQKLISEED LEIVLTQSPG TLSLSPGERA |
| | TLSCRASQSV GSSYLAWYQQ KPGQAPRLLI YGAFSRATGI PDRFSGSGSG |
| | TDFTLTISRL EPEDFAVYYC QQYGSSPWTF GQGTKVEIKG GGGSGGGGSG |
| | GGGSQVQLVE SGGGVVQPGR SLRLSCAASG FTFSSYTMHW VRQAPGKGLE |
| | WVTFISYDGN NKYYADSVKG RFTISRDNSK NTLYLQMNSL RAEDTAIYYC |
| | ARTGWLGPFD YWGQGTLVTV SSAAAGSGGS GILVKQSPML VAYDNAVNLS |
| | CKYSYNLESR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 51 | |
| vector clone: | pIB1019 |
| Sequence: | MALPVTALLL PLALLLHAAR PEQKLISEED LEVQLVESGG GVVRPGGSLR |
| | LSCVASGVTF DDYGMSWVRQ APGKGLEWVS GINWNGGDTD YSDSVKGRFT |
| | ISRDNAKNSL YLQMNSLRAE DTALYYCARD FYGSGSYYHV PFDYWGQGIL |
| | VTVSSGGGGS GGGGSGGGGS EIVLTQSPGT LSLSPGERAT LSCRASQSVS |
| | RSYLAWYQQK RGQAPRLLIY GASSRATGIP DRFSGDGSGT DFTLSISRLE |
| | PEDFAVYYCH QYDMSPFTFG PGTKVDIKAA AGSGGSGILV KQSPMLVAYD |
| | NAVNLSCKYS YNLESREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 52 | |
| vector clone: | pIB1020 |
| Sequence: | MALPVTALLL PLALLLHAAR PEQKLISEED LEIVLTQSPG TLSLSPGERA |
| | TLSCRASQSV SRSYLAWYQQ KRGQAPRLLI YGASSRATGI PDRFSGDGSG |
| | TDFTLSISRL EPEDFAVYYC HQYDMSPFTF GPGTKVDIKG GGGSGGGGSG |
| | GGGSEVQLVE SGGGVVRPGG SLRLSCVASG VTFDDYGMSW VRQAPGKGLE |
| | WVSGINWNGG DTDYSDSVKG RFTISRDNAK NSLYLQMNSL RAEDTALYYC |
| | ARDFYGSGSY YHVPFDYWGQ GILVTVSSAA AGSGGSGILV KQSPMLVAYD |
| | NAVNLSCKYS YNLFSREFRA SLHKGLDSAV EVCVVYGNYS QQLQVYSKTG |
| | FNCDGKLGNE SVTFYLQNLY VNQTDIYFCK IEVMYPPPYL DNEKSNGTII |
| | HVKGKHLCPS PLFPGPSKPF WVLVVVGGVL ACYSLLVTVA FIIFWVRSKR |
| | SRLLHSDYMN MTPRRPGPTR KHYQPYAPPR DFAAYRSKKV AKKPTNKAPH |
| | PKQEPQEINF PDDLPGSNTA APVQETLHGC QPVTQEDGKE SRISVQERQ |
| SEQ ID NO: 53 | |
| vector clone: | pIB1021 |
| Sequence: | MALPVTALLL PLALLLHAAR PEQKLISEED LQVQLVESGG GVVQPGRSLR |
| | LSCAASGFSF SSTYVCWVRQ APGKGLEWIA CIYTGDGTNY SASWAKGRET |
| | ISKDSSKNTV YLQMNSLRAE DTAVYFCARP DITYGFAINF WGPGTLVTVS |
| | SGGGGSGGGG SGGGGSDIQM TQSPSSLSAS VGDRVTIKCQ ASQSISSRLA |
| | WYQQKPGKPP KLLIYRASTL ASGVPSRFSG SGSGTDFTLT ISSLQPEDVA |
| | TYYCQCTGYG ISWPIGGGTK VEIKAAAGSG GSGILVKQSP MLVAYDNAVN |
| | LSCKYSYNLF SREFRASLHK GLDSAVEVCV VYGNYSQQLQ VYSKTGFNCD |
| | GKLGNESVTF YLQNLYVNQT DIYFCKIEVM YPPPYLDNEK SNGTIIHVKG |
| | KHLCPSPLFP GPSKPFWVLV VVGGVLACYS LLVTVAFIIF WVRSKRSRLL |
| | HSDYMNMTPR RPGPTRKHYQ PYAPPRDFAA YRSKKVAKKP TNKAPHPKQE |
| | PQEINFPDDL PGSNTAAPVQ ETLHGCQPVT QEDGKESRIS VQERQ |
| SEQ ID NO: 54 | |
| vector clone: | pIB1022 |
| Sequence: | MALPVTALLL PLALLLHAAR PEQKLISEED LDIQMTQSPS SLSASVGDRV |
| | TIKCQASQSI SSRLAWYQQK PGKPPKLLIY RASTLASGVP SRFSGSGSGT |
| | DFTLTISSLQ PEDVATYYCQ CTGYGISWPI GGGTKVEIKG GGGSGGGGSG |
| | GGGSQVQLVE SGGGVVQPGR SLRLSCAASG FSFSSTYVCW VRQAPGKGLE |
| | WIACIYTGDG TNYSASWAKG RFTISKDSSK NTVYLQMNSL RAEDTAVYFC |
| | ARPDITYGFA INFWGPGTLV TVSSAAAGSG GSGILVKQSP MLVAYDNAVN |
| | LSCKYSYNLF SREFRASLHK GLDSAVEVCV VYGNYSQQLQ VYSKTGFNCD |
| | GKLGNESVTF YLQNLYVNQT DIYFCKIEVM YPPPYLDNEK SNGTIIHVKG |
| | KHLCPSPLFP GPSKPFWVLV VVGGVLACYS LLVTVAFIIF WVRSKRSRLL |
| | HSDYMNMTPR RPGPTRKHYQ PYAPPRDFAA YRSKKVAKKP TNKAPHPKQE |
| | PQEINFPDDL PGSNTAAPVQ ETLHGCQPVT QEDGKESRIS VQERQ |
| SEQ ID NO: 55 | |
| vector clone: | pIB1023 |
| Sequence: | MALPVTALLL PLALLLHAAR PEQKLISEED LQVQLVQSGA EVKKPGASVK |
| | VSCKASGYTF TGYYMHWVRQ APGQGLEWMG WINPDSGGTN YAQKFQGRVT |
| | MTRDTSISTA YMELNRLRSD DTAVYYCARD QPLGYCTNGV CSYFDYWGQG |
| | TLVTVSSGGG GSGGGGSGGG GSDIQMTQSP SSVSASVGDR VTITCRASQG |
| | IYSWLAWYQQ KPGKAPNLLI YTASTLQSGV PSRFSGSGSG TDFTLTISSL |
| | QPEDFATYYC QQANIFPLTF GGGTKVEIKA AAGSGGSGIL VKQSPMLVAY |
| | DNAVNLSCKY SYNLFSREFR ASLHKGLDSA VEVCVVYGNY SQQLQVYSKT |
| | GFNCDGKLGN ESVTFYLQNL YVNQTDIYFC KIEVMYPPPY LDNEKSNGTI |
| | IHVKGKHLCP SPLFPGPSKP FWVLVVVGGV LACYSLLVTV AFIIFWVRSK |
| | RSRLLHSDYM NMTPRRPGPT RKHYQPYAPP RDFAAYRSKK VAKKPTNKAP |
| | HPKQEPQEIN FPDDLPGSNT AAPVQETLHG CQPVTQEDGK ESRISVQERQ |
| SEQ ID NO: 56 | |
| vector clone: | pIB1024 |
| Sequence: | MALPVTALLL PLALLLHAAR PEQKLISEED LDIQMTQSPS SVSASVGDRV |
| | TITCRASQGI YSWLAWYQQK PGKAPNLLIY TASTLQSGVP SRFSGSGSGT |
| | DFTLTISSLQ PEDFATYYCQ QANIFPLTFG GGTKVEIKGG GGSGGGGSGG |
| | GGSQVQLVQS GAEVKKPGAS VKVSCKASGY TFTGYYMHWV RQAPGQGLEW |
| | MGWINPDSGG TNYAQKFQGR VTMTRDTSIS TAYMELNRLR SDDTAVYYCA |
| | RDQPLGYCTN GVCSYFDYWG QGTLVTVSSA AAGSGGSGIL VKQSPMLVAY |
| | DNAVNLSCKY SYNLFSREFR ASLHKGLDSA VEVCVVYGNY SQQLQVYSKT |
| | GFNCDGKLGN ESVTFYLQNL YVNQTDIYFC KIEVMYPPPY LDNEKSNGTI |
| | IHVKGKHLCP SPLFPGPSKP FWVLVVVGGV LACYSLLVTV AFIIFWVRSK |
| | RSRLLHSDYM NMTPRRPGPT RKHYQPYAPP RDFAAYRSKK VAKKPTNKAP |
| | HPKQEPQEIN FPDDLPGSNT AAPVQETLHG CQPVTQEDGK ESRISVQERQ |
| SEQ ID NO: 57 | |
| vector clone: | pIB1025 |
| Sequence: | MALPVTALLL PLALLLHAAR PEQKLISEED LILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 58 | |
| vector clone: | pIB1026 |
| Sequence: | MEHSTFLSGL VLATLLSQVS PEQKLISEED LFKIPIEELE DRVFVNCNTS |
| | ITWVEGTVGT LLSDITRLDL GKRILDPRGI YRCNGTDIYK DKESTVQVHY |
| | RMCQSCVELD PATVAGIIVT DVIATLLLAL GVFCFARSKR SRLLHSDYMN |
| | MTPRRPGPTR KHYQPYAPPR DFAAYRSKKV AKKPTNKAPH PKQEPQEINF |
| | PDDLPGSNTA APVQETLHGC QPVTQEDGKE SRISVQERQ |
| SEQ ID NO: 59 | |
| vector clone: | pIB1027 |
| Sequence: | MQSGTHWRVL GLCLLSVGVW GQDYKDDDDK DGNEEMGGIT QTPYKVSISG |
| | TTVILTCPQY PGSEILWQHN DKNIGGDEDD KNIGSDEDHL SLKEFSELEQ |
| | SGYYVCYPRG SKPEDANFYL YLRARVCENC MEMDVMSVAT IVIVDICITG |
| | GLLLLVYYWS RSKRSRLLHS DYMNMTPRRP GPTRKHYQPY APPRDFAAYR |
| | SKKVAKKPTN KAPHPKQEPQ EINFPDDLPG SNTAAPVQET LHGCQPVTQE |
| | DGKESRISVQ ERQ |
| SEQ ID NO: 60 | |
| vector clone: | pIB1028 |
| Sequence: | MEQGKGLAVL ILAIILLQGT LAEQKLISEE DLQSIKGNHL VKVYDYQEDG |
| | SVLLTCDAEA KNITWFKDGK MIGFLTEDKK KWNLGSNAKD PRGMYQCKGS |
| | QNKSKPLQVY YRMCQNCIEL NAATISGFLF AEIVSIFVLA VGVYFIARSK |
| | RSRLLHSDYM NMTPRRPGPT RKHYQPYAPP RDFAAYRSKK VAKKPTNKAP |
| | HPKQEPQEIN FPDDLPGSNT AAPVQETLHG CQPVTQEDGK ESRISVQERQ |
| SEQ ID NO: 61 | |
| vector clone: | pIB1029 |
| Sequence: | MKWKALFTAA ILQAQLPITE AQSFGLLDPK LCYLLDGILF IYGVILTALF |
| | LRSKRSRLLH SDYMNMTPRR PGPTRKHYQP YAPPRDFAAY RSKKVAKKPT |
| | NKAPHPKQEP QEINFPDDLP GSNTAAPVQE TLHGCQPVTQ EDGKESRISV |
| | QERQEQKLIS EEDL |
| SEQ ID NO: 62 | |
| vector clone: | pIB1030 |
| Sequence: | MALPVTALLL PLALLLHAAR PEQKLISEED LSQPHTKPSV FVMKNGTNVA |
| | CLVKEFYPKD IRINLVSSKK ITEFDPAIVI SPSGKYNAVK LGKYEDSNSV |
| | TCSVQHDNKT VHSTDFEVKT DSTDHVKPKE TENTKQPSKS CHKPKAIVHT |
| | EKVNMMSLTV LGLRMLFAKT VAVNFLLTAK LFFLRSKRSR LLHSDYMNMT |
| | PRRPGPTRKH YQPYAPPRDF AAYRSKKVAK KPTNKAPHPK QEPQEINFPD |
| | DLPGSNTAAP VQETLHGCQP VTQEDGKESR ISVQERQ |
| SEQ ID NO: 63 | |
| vector clone: | pIB1031 |
| Sequence: | MALPVTALLL PLALLLHAAR PDYKDDDDKD KQLDADVSPK PTIFLPSIAE |
| | TKLQKAGTYL CLLEKFFPDV IKIHWQEKKS NTILGSQEGN TMKTNDTYMK |
| | FSWLTVPEKS LDKEHRCIVR HENNKNGVDQ EIIFPPIKTD VITMDPKDNC |
| | SKDANDTLLL QLTNTSAYYM YLLLLLKSVV YFAIITCCLL RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID NO: 64 | |
| vector clone: | pIB1032 |
| Sequence: | MALPVTALLL PLALLLHAAR PEQKLISEED LIQNPEPAVY QLKDPRSQDS |
| | TLCLFTDFDS QINVPKTMES GTFITDKCVL DMKAMDSKSN GAIAWSNQTS |
| | FTCQDIFKET NATYPSSDVP CDATLTEKSF ETDMNLNFQN LLVIVLRILL |
| | LKVAGFNLLM TLRLWSSRSK RSRLLHSDYM NMTPRRPGPT RKHYQPYAPP |
| | RDFAAYRSKK VAKKPTNKAP HPKQEPQEIN FPDDLPGSNT AAPVQETLHG |
| | CQPVTQEDGK ESRISVQERQ |
| SEQ ID NO: 65 | |
| vector clone: | pIB1033 |
| Sequence: | MALPVTALLL PLALLLHAAR PDYKDDDDKV LTPPKVSLFE PSKAEIANKQ |
| | KATLVCLARG FFPDHVELSW WVNGKEVHSG VCTDPQAYKE SNYSYCLSSR |
| | LRVSATFWHN PRNHFRCQVQ FHGLSEEDKW PEGSPKPVTQ NISAEAWGRA |
| | DCGITSASYQ QGVLSATILY EILLGKATLY AVLVSTLVVM AMVKRKNSRS |
| | KRSRLLHSDY MNMTPRRPGP TRKHYQPYAP PRDFAAYRSK KVAKKPTNKA |
| | PHPKQEPQEI NFPDDLPGSN TAAPVQETLH GCQPVTQEDG KESRISVQER |
| | Q |
| SEQ ID NO: 66 | |
| vector clone: | pIB1046 |
| Sequence: | MALPVTALLL PLALLLHAAR PEQKLISEED LSQPHTKPSV FVMKNGTNVA |
| | CLVKEFYPKD IRINLVSSKK ITEFDPAIVI SPSGKYNAVK LGKYEDSNSV |
| | TCSVQHDNKT VHSTDFEVKT DSTDHVKPKE TENTKQPSKS CHKPKAIVHT |
| | EKVNMMSLTV LGLRMLFAKT VAVNFLLTAK LFFLRSKRSR LLHSDYMNMT |
| | PRRPGPTRKH YQPYAPPRDF AAYRSKKVAK KPTNKAPHPK QEPQEINFPD |
| | DLPGSNTAAP VQETLHGCQP VTQEDGKESR ISVQERQRAK RGSGEGRGSL |
| | LTCGDVEENP GPMALPVTAL LLPLALLLHA ARPDYKDDDD KDKQLDADVS |
| | PKPTIFLPSI AETKLQKAGT YLCLLEKFFP DVIKIHWQEK KSNTILGSQE |
| | GNTMKTNDTY MKFSWLTVPE KSLDKEHRCI VRHENNKNGV DQEIIFPPIK |
| | TDVITMDPKD NCSKDANDTL LLQLTNTSAY YMYLLLLLKS VVYFAIITCC |
| | LLRSKRSRLL HSDYMNMTPR RPGPTRKHYQ PYAPPRDFAA YRSKKVAKKP |
| | TNKAPHPKQE PQEINFPDDL PGSNTAAPVQ ETLHGCQPVT QEDGKESRIS |
| | VQERQ |
| SEQ ID NO: 67 | |
| vector clone: | pIB1047 |
| Sequence: | MALPVTALLL PLALLLHAAR PEQKLISEED LIQNPEPAVY QLKDPRSQDS |
| | TLCLFTDFDS QINVPKTMES GTFITDKCVL DMKAMDSKSN GAIAWSNQTS |
| | FTCQDIFKET NATYPSSDVP CDATLTEKSF ETDMNLNFQN LLVIVLRILL |
| | LKVAGENLLM TLRLWSSRSK RSRLLHSDYM NMTPRRPGPT RKHYQPYAPP |
| | RDFAAYRSKK VAKKPTNKAP HPKQEPQEIN FPDDLPGSNT AAPVQETLHG |
| | CQPVTQEDGK ESRISVQERQ RAKRGSGEGR GSLLTCGDVE ENPGPMALPV |
| | TALLLPLALL LHAARPDYKD DDDKVLTPPK VSLFEPSKAE IANKQKATLV |
| | CLARGFFPDH VELSWWVNGK EVHSGVCTDP QAYKESNYSY CLSSRLRVSA |
| | TFWHNPRNHF RCQVQFHGLS EEDKWPEGSP KPVTQNISAE AWGRADCGIT |
| | SASYQQGVLS ATILYEILLG KATLYAVLVS TLVVMAMVKR KNSRSKRSRL |
| | LHSDYMNMTP RRPGPTRKHY QPYAPPRDFA AYRSKKVAKK PTNKAPHPKQ |
| | EPQEINFPDD LPGSNTAAPV QETLHGCQPV TQEDGKESRI SVQERQ |
| SEQ ID NO: 68 | |
| vector clone: | pIB1048 |
| Sequence: | MEHSTELSGL VLATLLSQVS PEQKLISEED LFKIPIEELE DRVFVNCNTS |
| | ITWVEGTVGT LLSDITRLDL GKRILDPRGI YRCNGTDIYK DKESTVQVHY |
| | RMCQSCVELD PATVAGIIVT DVIATLLLAL GVFCFARSKR SRLLHSDYMN |
| | MTPRRPGPTR KHYQPYAPPR DFAAYRSKKV AKKPTNKAPH PKQEPQEINF |
| | PDDLPGSNTA APVQETLHGC QPVTQEDGKE SRISVQERQR AKRGSGEGRG |
| | SLLTCGDVEE NPGPMQSGTH WRVLGLCLLS VGVWGQDYKD DDDKDGNEEM |
| | GGITQTPYKV SISGTTVILT CPQYPGSEIL WQHNDKNIGG DEDDKNIGSD |
| | EDHLSLKEFS ELEQSGYYVC YPRGSKPEDA NFYLYLRARV CENCMEMDVM |
| | SVATIVIVDI CITGGLLLLV YYWSRSKRSR LLHSDYMNMT PRRPGPTRKH |
| | YQPYAPPRDF AAYRSKKVAK KPTNKAPHPK QEPQEINFPD DLPGSNTAAP |
| | VQETLHGCQP VTQEDGKESR ISVQERQ |
| SEQ ID NO: 69 | |
| vector clone: | pIB1049 |
| Sequence: | MEQGKGLAVL ILAIILLQGT LAEQKLISEE DLQSIKGNHL VKVYDYQEDG |
| | SVLLTCDAEA KNITWFKDGK MIGFLTEDKK KWNLGSNAKD PRGMYQCKGS |
| | QNKSKPLQVY YRMCQNCIEL NAATISGFLF AEIVSIFVLA VGVYFIARSK |
| | RSRLLHSDYM NMTPRRPGPT RKHYQPYAPP RDFAAYRSKK VAKKPTNKAP |
| | HPKQEPQEIN FPDDLPGSNT AAPVQETLHG CQPVTQEDGK ESRISVQERQ |
| | RAKRGSGMQS GTHWRVLGLC LLSVGVWGQD YKDDDDKDGN EEMGGITQTP |
| | YKVSISGTTV ILTCPQYPGS EILWQHNDKN IGGDEDDKNI GSDEDHLSLK |
| | EFSELEQSGY YVCYPRGSKP EDANFYLYLR ARVCENCMEM DVMSVATIVI |
| | VDICITGGLL LLVYYWSRSK RSRLLHSDYM NMTPRRPGPT RKHYQPYAPP |
| | RDFAAYRSKK VAKKPTNKAP HPKQEPQEIN FPDDLPGSNT AAPVQETLHG |
| | CQPVTQEDGK ESRISVQERQ |
| SEQ ID NO: 70 | |
| vector clone: | pIB1050 |
| Sequence: | MEHSTFLSGL VLATLLSQVS PEQKLISEED LFKIPIEELE DRVFVNCNTS |
| | ITWVEGTVGT LLSDITRLDL GKRILDPRGI YRCNGTDIYK DKESTVQVHY |
| | RMCQSCVELD PATVAGIIVT DVIATLLLAL GVFCFAGHET GRLSGAADTQ |
| | ALLRNDQVYQ PLRDRDDAQY SHLGGNWARN KRSKRSRLLH SDYMNMTPRR |
| | PGPTRKHYQP YAPPRDFAAY RSKKVAKKPT NKAPHPKQEP QEINFPDDLP |
| | GSNTAAPVQE TLHGCQPVTQ EDGKESRISV QERQRAKRGS GEGRGSLLTC |
| | GDVEENPGPM QSGTHWRVLG LCLLSVGVWG QDYKDDDDKD GNEEMGGITQ |
| | TPYKVSISGT TVILTCPQYP GSEILWQHND KNIGGDEDDK NIGSDEDHLS |
| | LKEFSELEQS GYYVCYPRGS KPEDANFYLY LRARVCENCM EMDVMSVATI |
| | VIVDICITGG LLLLVYYWSK NRKAKAKPVT RGAGAGGRQR GQNKERPPPV |
| | PNPDYEPIRK GQRDLYSGLN QRRIRSKRSR LLHSDYMNMT PRRPGPTRKH |
| | YQPYAPPRDF AAYRSKKVAK KPTNKAPHPK QEPQEINFPD DLPGSNTAAP |
| | VQETLHGCQP VTQEDGKESR ISVQERQ |
| SEQ ID NO: 71 | |
| vector clone: | pIB1051 |
| Sequence: | MEHSTFLSGL VLATLLSQVS PEQKLISEED LFKIPIEELE DRVFVNCNTS |
| | ITWVEGTVGT LLSDITRLDL GKRILDPRGI YRCNGTDIYK DKESTVQVHY |
| | RMCQSCVELD PATVAGIIVT DVIATLLLAL GVFCFAGHET GRLSGAADTQ |
| | ALLRNDQVYQ PLRDRDDAQY SHLGGNWARN KRAKRGSGEG RGSLLTCGDV |
| | EENPGPMQSG THWRVLGLCL LSVGVWGQDY KDDDDKDGNE EMGGITQTPY |
| | KVSISGTTVI LTCPQYPGSE ILWQHNDKNI GGDEDDKNIG SDEDHLSLKE |
| | FSELEQSGYY VCYPRGSKPE DANFYLYLRA RVCENCMEMD VMSVATIVIV |
| | DICITGGLLL LVYYWSKNRK AKAKPVTRGA GAGGRQRGQN KERPPPVPNP |
| | DYEPIRKGQR DLYSGLNQRR I |
| SEQ ID NO: 72 | |
| vector clone: | pIB1052 |
| Sequence: | MEHSTFLSGL VLATLLSQVS PEQKLISEED LFKIPIEELE DRVFVNCNTS |
| | ITWVEGTVGT LLSDITRLDL GKRILDPRGI YRCNGTDIYK DKESTVQVHY |
| | RMCQSCVELD PATVAGIIVT DVIATLLLAL GVFCFARAKR GSGEGRGSLL |
| | TCGDVEENPG PMQSGTHWRV LGLCLLSVGV WGQDYKDDDD KDGNEEMGGI |
| | TQTPYKVSIS GTTVILTCPQ YPGSEILWQH NDKNIGGDED DKNIGSDEDH |
| | LSLKEFSELE QSGYYVCYPR GSKPEDANFY LYLRARVCEN CMEMDVMSVA |
| | TIVIVDICIT GGLLLLVYYW S |
| SEQ ID NO: 73 | |
| vector clone: | pIB1053 |
| Sequence: | MEQGKGLAVL ILAIILLQGT LAEQKLISEE DLQSIKGNHL VKVYDYQEDG |
| | SVLLTCDAEA KNITWFKDGK MIGFLTEDKK KWNLGSNAKD PRGMYQCKGS |
| | QNKSKPLQVY YRMCQNCIEL NAATISGFLF AEIVSIFVLA VGVYFIAGQD |
| | GVRQSRASDK QTLLPNDQLY QPLKDREDDQ YSHLQGNQLR RNRSKRSRLL |
| | HSDYMNMTPR RPGPTRKHYQ PYAPPRDFAA YRSKKVAKKP TNKAPHPKQE |
| | PQEINFPDDL PGSNTAAPVQ ETLHGCQPVT QEDGKESRIS VQERQRAKRG |
| | SGEGRGSLLT CGDVEENPGP MQSGTHWRVL GLCLLSVGVW GQDYKDDDDK |
| | DGNEEMGGIT QTPYKVSISG TTVILTCPQY PGSEILWQHN DKNIGGDEDD |
| | KNIGSDEDHL SLKEFSELEQ SGYYVCYPRG SKPEDANFYL YLRARVCENC |
| | MEMDVMSVAT IVIVDICITG GLLLLVYYWS KNRKAKAKPV TRGAGAGGRQ |
| | RGQNKERPPP VPNPDYEPIR KGQRDLYSGL NQRRIRSKRS RLLHSDYMNM |
| | TPRRPGPTRK HYQPYAPPRD FAAYRSKKVA KKPTNKAPHP KQEPQEINFP |
| | DDLPGSNTAA PVQETLHGCQ PVTQEDGKES RISVQERQ |
| SEQ ID NO: 74 | |
| vector clone: | pIB1054 |
| Sequence: | MEQGKGLAVL ILAIILLQGT LAEQKLISEE DLQSIKGNHL VKVYDYQEDG |
| | SVLLTCDAEA KNITWFKDGK MIGFLTEDKK KWNLGSNAKD PRGMYQCKGS |
| | QNKSKPLQVY YRMCQNCIEL NAATISGFLF AEIVSIFVLA VGVYFIAGQD |
| | GVRQSRASDK QTLLPNDQLY QPLKDREDDQ YSHLQGNQLR RNRAKRGSGE |
| | GRGSLLTCGD VEENPGPMQS GTHWRVLGLC LLSVGVWGQD YKDDDDKDGN |
| | EEMGGITQTP YKVSISGTTV ILTCPQYPGS EILWQHNDKN IGGDEDDKNI |
| | GSDEDHLSLK EFSELEQSGY YVCYPRGSKP EDANFYLYLR ARVCENCMEM |
| | DVMSVATIVI VDICITGGLL LLVYYWSKNR KAKAKPVTRG AGAGGRQRGQ |
| | NKERPPPVPN PDYEPIRKGQ RDLYSGLNQR RI |
| SEQ ID NO: 75 | |
| vector clone: | pIB1055 |
| Sequence: | MEQGKGLAVL ILAIILLQGT LAEQKLISEE DLQSIKGNHL VKVYDYQEDG |
| | SVLLTCDAEA KNITWFKDGK MIGFLTEDKK KWNLGSNAKD PRGMYQCKGS |
| | QNKSKPLQVY YRMCQNCIEL NAATISGFLF AEIVSIFVLA VGVYFIARAK |
| | RGSGEGRGSL LTCGDVEENP GPMQSGTHWR VLGLCLLSVG VWGQDYKDDD |
| | DKDGNEEMGG ITQTPYKVSI SGTTVILTCP QYPGSEILWQ HNDKNIGGDE |
| | DDKNIGSDED HLSLKEFSEL EQSGYYVCYP RGSKPEDANF YLYLRARVCE |
| | NCMEMDVMSV ATIVIVDICI TGGLLLLVYY WS |
| SEQ ID NO: 76 | |
| vector clone: 77 | pIB1056 |
| Sequence: | MKWKALFTAA ILQAQLPITE AQSFGLLDPK LCYLLDGILF IYGVILTALF |
| | LRVKFSRSAD APAYQQGQNQ LYNELNLGRR EEYDVLDKRR GRDPEMGGKP |
| | QRRKNPQEGL YNELQKDKMA EAYSEIGMKG ERRRGKGHDG LYQGLSTATK |
| | DTYDALHMQA LPPRRSKRSR LLHSDYMNMT PRRPGPTRKH YQPYAPPRDF |
| | AAYRSKKVAK KPTNKAPHPK QEPQEINFPD DLPGSNTAAP VQETLHGCQP |
| | VTQEDGKESR ISVQERQEQK LISEEDL |
| SEQ ID NO: 78 | |
| vector clone: | pIB1057 |
| Sequence: | MKWKALFTAA ILQAQLPITE AQSFGLLDPK LCYLLDGILF IYGVILTALF |
| | LRVKFSRSAD APAYQQGQNQ LYNELNLGRR EEYDVLDKRR GRDPEMGGKP |
| | QRRKNPQEGL YNELQKDKMA EAYSEIGMKG ERRRGKGHDG LYQGLSTATK |
| | DTYDALHMQA LPPREQKLIS EEDL |
| SEQ ID NO: 79 | |
| vector clone: | pIB1058 |
| Sequence: | MKWKALFTAA ILQAQLPITE AQSFGLLDPK LCYLLDGILF IYGVILTALF |
| | LEQKLISEED L |
| SEQ ID NO: 80 | |
| vector clone: | pIB1059 |
| Sequence: | MKWKALFTAA ILQAQLPITE AQSFGLLDPK LCYLLDGILF IYGVILTALF |
| | LRVKFSRSAD APAYQQGQNQ LYNELNLGRR EEYDVLDKRR GRDPEMGGKP |
| | QRRKNPQEGL YNELQKDKMA EAYSEIGMKG ERRRGKGHDG LYQGLSTATK |
| | DTYDALHMQA LPPRRVKFSR SADAPAYQQG QNQLYNELNL GRREEYDVLD |
| | KRRGRDPEMG GKPQRRKNPQ EGLYNELQKD KMAEAYSEIG MKGERRRGKG |
| | HDGLYQGLST ATKDTYDALH MQALPPRRSK RSRLLHSDYM NMTPRRPGPT |
| | RKHYQPYAPP RDFAAYRSKK VAKKPTNKAP HPKQEPQEIN FPDDLPGSNT |
| | AAPVQETLHG CQPVTQEDGK ESRISVQERQ EQKLISEEDL |
| SEQ ID NO: 81 | |
| vector clone: | pIB1060 |
| Sequence: | MKWKALFTAA ILQAQLPITE AQSFGLLDPK LCYLLDGILF IYGVILTALF |
| | LRVKFSRSAD APAYQQGQNQ LYNELNLGRR EEYDVLDKRR GRDPEMGGKP |
| | QRRKNPQEGL YNELQKDKMA EAYSEIGMKG ERRRGKGHDG LYQGLSTATK |
| | DTYDALHMQA LPPRRVKFSR SADAPAYQQG QNQLYNELNL GRREEYDVLD |
| | KRRGRDPEMG GKPQRRKNPQ EGLYNELQKD KMAEAYSEIG MKGERRRGKG |
| | HDGLYQGLST ATKDTYDALH MQALPPREQK LISEEDL |
| SEQ ID NO: 82 | |
| vector clone: | pIB1061 |
| Sequence: | MKWKALFTAA ILQAQLPITE AQSFGLLDPK LCYLLDGILF IYGVILTALF |
| | LRSKRSRLLH SDYMNMTPRR PGPTRKHYQP YAPPRDFAAY RDKKVAKKPT |
| | NKAPHPKQEP QEINFPDDLP GSNTAAPVQE TLHGCQPVTQ EDGKESRISV |
| | QERQRVKFSR SADAPAYQQG QNQLYNELNL GRREEYDVLD KRRGRDPEMG |
| | GKPQRRKNPQ EGLYNELQKD KMAEAYSEIG MKGERRRGKG HDGLYQGLST |
| | ATKDTYDALH MQALPPRRVK FSRSADAPAY QQGQNQLYNE LNLGRREEYD |
| | VLDKRRGRDP EMGGKPQRRK NPQEGLYNEL QKDKMAEAYS EIGMKGERRR |
| | GKGHDGLYQG LSTATKDTYD ALHMQALPPR EQKLISEEDL |
| SEQ ID NO: 83 | |
| vector clone: | pIB1062 |
| Sequence: | MKWKALFTAA ILQAQLPITE AQSFGLLDPK LCYLLDGILF IYGVILTALF |
| | LRSKRSRLLH SDYMNMTPRR PGPTRKHYQP YAPPRDFAAY RSKKVAKKPT |
| | NKAPHPKQEP QEINFPDDLP GSNTAAPVQE TLHGCQPVTQ EDGKESRISV |
| | QERQRVKFSR SADAPAYQQG QNQLYNELNL GRREEYDVLD KRRGRDPEMG |
| | GKPQRRKNPQ EGLYNELQKD KMAEAYSEIG MKGERRRGKG HDGLYQGLST |
| | ATKDTYDALH MQALPPREQK LISEEDL |
| SEQ ID NO: 84 | |
| vector clone: | pIB1063 |
| Sequence: | MGHTRRQGTS PSKCPYLNFF QLLVLAGLSH FCSGEQKLIS EEDLVIHVTK |
| | EVKEVATLSC GHNVSVEELA QTRIYWQKEK KMVLTMMSGD MNIWPEYKNR |
| | TIFDITNNLS IVILALRPSD EGTYECVVLK YEKDAFKREH LAEVTLSVKA |
| | DFPTPSISDF EIPTSNIRRI ICSTSGGFPE PHLSWLENGE ELNAINTTVS |
| | QDPETELYAV SSKLDFNMTT NHSFMCLIKY GHLRVNQTFN WNTTKQEHFP |
| | DNLLPSWAIT LISVNGIFVI CCL |
| SEQ ID NO: 85 | |
| vector clone: | pIB1064 |
| Sequence: | MGCGCSSHPE DDWMENIDVC ENCHYPIVPL DGKGTLLIRN GSEVRDPLVT |
| | YEGSNPPASP LQDNLVIALH SYEPSHDGDL GFEKGEQLRI LEQSGEWWKA |
| | QSLTTGQEGF IPFNFVAKAN SLEPEPWFFK NLSRKDAERQ LLAPGNTHGS |
| | FLIRESESTA GSFSLSVRDF DQNQGEVVKH YKIRNLDNGG FYISPRITFP |
| | GLHELVRHYT NASDGLCTRL SRPCQTQKPQ KPWWEDEWEV PRETLKLVER |
| | LGAGQFGEVW MGYYNGHTKV AVKSLKQGSM SPDAFLAEAN LMKQLQHQRL |
| | VRLYAVVTQE PIYIITEYME NGSLVDFLKT PSGIKLTINK LLDMAAQIAE |
| | GMAFIEERNY IHRDLRAANI LVSDTLSCKI ADFGLARLIE DNEYTAREGA |
| | KFPIKWTAPE AINYGTFTIK SDVWSFGILL TEIVTHGRIP YPGMTNPEVI |
| | QNLERGYRMV RPDNCPEELY QLMRLCWKER PEDRPTFDYL RSVLEDFFTA |
| | TEGQYQPQPE QKLISEEDL |
| SEQ ID NO: 86 | |
| vector clone: | pIB1065 |
| Sequence: | MGCGCSSHPE DDWMENIDVC ENCHYPIVPL DGKGTLLIRN GSEVRDPLVT |
| | YEGSNPPASP LQDNLVIALH SYEPSHDGDL GFEKGEQLRI LEQSGEWWKA |
| | QSLTTGQEGF IPFNFVAKAN SLEPEPWFFK NLSRKDAERQ LLAPGNTHGS |
| | FLIRESESTA GSFSLSVRDF DQNQGEVVKH YKIRNLDNGG FYISPRITFP |
| | GLHELVRHYT NASDGLCTRL SRPCQTQKPQ KPWWEDEWEV PRETLKLVER |
| | LGAGQFGEVW MGYYNGHTKV AVKSLKQGSM SPDAFLAEAN LMKQLQHQRL |
| | VRLYAVVTQE PIYIITEYME NGSLVDFLKT PSGIKLTINK LLDMAAQIAE |
| | GMAFIEERNY IHRDLRAANI LVSDTLSCKI ADFGLARLIE DNEYTAREGA |
| | KFPIKWTAPE AINYGTFTIK SDVWSFGILL TEIVTHGRIP YPGMTNPEVI |
| | QNLERGYRMV RPDNCPEELY QLMRLCWKER PEDRPTFDYL RSVLEDFFTA |
| | TEGQFQPQPE QKLISEEDL |
| SEQ ID NO: 87 | |
| vector clone: | pIB1066 |
| Sequence: | MGHTRRQGTS PSKCPYLNFF QLLVLAGLSH FCSGEQKLIS EEDLVIHVTK |
| | EVKEVATLSC GHNVSVEELA QTRIYWQKEK KMVLTMMSGD MNIWPEYKNR |
| | TIFDITNNLS IVILALRPSD EGTYECVVLK YEKDAFKREH LAEVTLSVKA |
| | DFPTPSISDF EIPTSNIRRI ICSTSGGFPE PHLSWLENGE ELNAINTTVS |
| | QDPETELYAV SSKLDFNMTT NHSFMCLIKY GHLRVNQTFN WNTTKQEHFP |
| | DNLLPSWAIT LISVNGIFVI CCLGCGCSSH PEDDWMENID VCENCHYPIV |
| | PLDGKGTLLI RNGSEVRDPL VTYEGSNPPA SPLQDNLVIA LHSYEPSHDG |
| | DLGFEKGEQL RILEQSGEWW KAQSLTTGQE GFIPFNFVAK ANSLEPEPWF |
| | FKNLSRKDAE RQLLAPGNTH GSFLIRESES TAGSFSLSVR DFDQNQGEVV |
| | KHYKIRNLDN GGFYISPRIT FPGLHELVRH YTNASDGLCT RLSRPCQTQK |
| | PQKPWWEDEW EVPRETLKLV ERLGAGQFGE VWMGYYNGHT KVAVKSLKQG |
| | SMSPDAFLAE ANLMKQLQHQ RLVRLYAVVT QEPIYIITEY MENGSLVDFL |
| | KTPSGIKLTI NKLLDMAAQI AEGMAFIEER NYIHRDLRAA NILVSDTLSC |
| | KIADFGLARL IEDNEYTARE GAKFPIKWTA PEAINYGTFT IKSDVWSFGI |
| | LLTEIVTHGR IPYPGMTNPE VIQNLERGYR MVRPDNCPEE LYQLMRLCWK |
| | ERPEDRPTFD YLRSVLEDFF TATEGQYQPQ P |
| SEQ ID NO: 88 | |
| vector clone: | pIB1067 |
| Sequence: | MGHTRRQGTS PSKCPYLNFF QLLVLAGLSH FCSGEQKLIS EEDLVIHVTK |
| | EVKEVATLSC GHNVSVEELA QTRIYWQKEK KMVLTMMSGD MNIWPEYKNR |
| | TIFDITNNLS IVILALRPSD EGTYECVVLK YEKDAFKREH LAEVTLSVKA |
| | DFPTPSISDF EIPTSNIRRI ICSTSGGFPE PHLSWLENGE ELNAINTTVS |
| | QDPETELYAV SSKLDFNMTT NHSFMCLIKY GHLRVNQTFN WNTTKQEHFP |
| | DNLLPSWAIT LISVNGIFVI CCLGCGCSSH PEDDWMENID VCENCHYPIV |
| | PLDGKGTLLI RNGSEVRDPL VTYEGSNPPA SPLQDNLVIA LHSYEPSHDG |
| | DLGFEKGEQL RILEQSGEWW KAQSLTTGQE GFIPFNFVAK ANSLEPEPWF |
| | FKNLSRKDAE RQLLAPGNTH GSFLIRESES TAGSFSLSVR DFDQNQGEVV |
| | KHYKIRNLDN GGFYISPRIT FPGLHELVRH YTNASDGLCT RLSRPCQTQK |
| | PQKPWWEDEW EVPRETLKLV ERLGAGQFGE VWMGYYNGHT KVAVKSLKQG |
| | SMSPDAFLAE ANLMKQLQHQ RLVRLYAVVT QEPIYIITEY MENGSLVDFL |
| | KTPSGIKLTI NKLLDMAAQI AEGMAFIEER NYIHRDLRAA NILVSDTLSC |
| | KIADFGLARL IEDNEYTARE GAKFPIKWTA PEAINYGTFT IKSDVWSFGI |
| | LLTEIVTHGR IPYPGMTNPE VIQNLERGYR MVRPDNCPEE LYQLMRLCWK |
| | ERPEDRPTFD YLRSVLEDFF TATEGQYQPQ PRSKRSRLLH SDYMNMTPRR |
| | PGPTRKHYQP YAPPRDFAAY RSKKVAKKPT NKAPHPKQEP QEINFPDDLP |
| | GSNTAAPVQE TLHGCQPVTQ EDGKESRISV QERQ |
| SEQ ID NO: 89 | |
| vector clone: | pIB1068 |
| Sequence: | MGHTRRQGTS PSKCPYLNFF QLLVLAGLSH FCSGEQKLIS EEDLVIHVTK |
| | EVKEVATLSC GHNVSVEELA QTRIYWQKEK KMVLTMMSGD MNIWPEYKNR |
| | TIFDITNNLS IVILALRPSD EGTYECVVLK YEKDAFKREH LAEVTLSVKA |
| | DFPTPSISDF EIPTSNIRRI ICSTSGGFPE PHLSWLENGE ELNAINTTVS |
| | QDPETELYAV SSKLDFNMTT NHSFMCLIKY GHLRVNQTFN WNTTKQEHFP |
| | DNLLPSWAIT LISVNGIFVI CCLGCGCSSH PEDDWMENID VCENCHYPIV |
| | PLDGKGTLLI RNGSEVRDPL VTYEGSNPPA SPLQDNLVIA LHSYEPSHDG |
| | DLGFEKGEQL RILEQSGEWW KAQSLTTGQE GFIPFNFVAK ANSLEPEPWF |
| | FKNLSRKDAE RQLLAPGNTH GSFLIRESES TAGSFSLSVR DFDQNQGEVV |
| | KHYKIRNLDN GGFYISPRIT FPGLHELVRH YTNASDGLCT RLSRPCQTQK |
| | PQKPWWEDEW EVPRETLKLV ERLGAGQFGE VWMGYYNGHT KVAVKSLKQG |
| | SMSPDAFLAE ANLMKQLQHQ RLVRLYAVVT QEPIYIITEY MENGSLVDFL |
| | KTPSGIKLTI NKLLDMAAQI AEGMAFIEER NYIHRDLRAA NILVSDTLSC |
| | KIADFGLARL IEDNEYTARE GAKFPIKWTA PEAINYGTFT IKSDVWSFGI |
| | LLTEIVTHGR IPYPGMTNPE VIQNLERGYR MVRPDNCPEE LYQLMRLCWK |
| | ERPEDRPTFD YLRSVLEDFF TATEGQFQPQ P |
| SEQ ID NO: 90 | |
| vector clone: | pIB1069 |
| Sequence: | MGHTRRQGTS PSKCPYLNFF QLLVLAGLSH FCSGEQKLIS EEDLVIHVTK |
| | EVKEVATLSC GHNVSVEELA QTRIYWQKEK KMVLTMMSGD MNIWPEYKNR |
| | TIFDITNNLS IVILALRPSD EGTYECVVLK YEKDAFKREH LAEVTLSVKA |
| | DFPTPSISDF EIPTSNIRRI ICSTSGGFPE PHLSWLENGE ELNAINTTVS |
| | QDPETELYAV SSKLDFNMTT NHSFMCLIKY GHLRVNQTFN WNTTKQEHFP |
| | DNLLPSWAIT LISVNGIFVI CCLGCGCSSH PEDDWMENID VCENCHYPIV |
| | PLDGKGTLLI RNGSEVRDPL VTYEGSNPPA SPLQDNLVIA LHSYEPSHDG |
| | DLGFEKGEQL RILEQSGEWW KAQSLTTGQE GFIPFNFVAK ANSLEPEPWF |
| | FKNLSRKDAE RQLLAPGNTH GSFLIRESES TAGSFSLSVR DFDQNQGEVV |
| | KHYKIRNLDN GGFYISPRIT FPGLHELVRH YTNASDGLCT RLSRPCQTQK |
| | PQKPWWEDEW EVPRETLKLV ERLGAGQFGE VWMGYYNGHT KVAVKSLKQG |
| | SMSPDAFLAE ANLMKQLQHQ RLVRLYAVVT QEPIYIITEY MENGSLVDFL |
| | KTPSGIKLTI NKLLDMAAQI AEGMAFIEER NYIHRDLRAA NILVSDTLSC |
| | KIADFGLARL IEDNEYTARE GAKFPIKWTA PEAINYGTFT IKSDVWSFGI |
| | LLTEIVTHGR IPYPGMTNPE VIQNLERGYR MVRPDNCPEE LYQLMRLCWK |
| | ERPEDRPTFD YLRSVLEDFF TATEGQFQPQ PRSKRSRLLH SDYMNMTPRR |
| | PGPTRKHYQP YAPPRDFAAY RSKKVAKKPT NKAPHPKQEP QEINFPDDLP |
| | GSNTAAPVQE TLHGCQPVTQ EDGKESRISV QERQ |
| SEQ ID NO: 91 | |
| vector clone: | pIB1070 |
| Sequence: | MALPVTALLL PLALLLHAAR PEQKLISEED LMEEAILVPC VLGLLLLPIL |
| | AMLMALCVHC HRLPGSYDST SSDSLYPRGI QFKRPHTVAP WPPAYPPVTS |
| | YPPLSQPDLL PIPRSPQPLG GSHRTPSSRR DSDGANSVAS YENEGASGIR |
| | GAQAGWGVWG PSWTRLTPVS LPPEPACEDA DEDEDDYHNP GYLVVLPDST |
| | PATSTAAPSA PALSTPGIRD SAFSMESIDD YVNVPESGES AEASLDGSRE |
| | YVNVSQELHP GAAKTEPAAL SSQEAEEVEE EGAPDYENLQ ELN |
| SEQ ID NO: 92 | |
| vector clone: | pIB1071 |
| Sequence: | MALPVTALLL PLALLLHAAR PEQKLISEED LMEEAILVPC VLGLLLLPIL |
| | AMLMALCVHC HRLPGSYDST SSDSLYPRGI QFKRPHTVAP WPPAYPPVTS |
| | YPPLSQPDLL PIPRSPQPLG GSHRTPSSRR DSDGANSVAS YENEGASGIR |
| | GAQAGWGVWG PSWTRLTPVS LPPEPACEDA DEDEDDYHNP GYLVVLPDST |
| | PATSTAAPSA PALSTPGIRD SAFSMESIDD YVNVPESGES AEASLDGSRE |
| | YVNVSQELHP GAAKTEPAAL SSQEAEEVEE EGAPDYENLQ ELNRSKRSRL |
| | LHSDYMNMTP RRPGPTRKHY QPYAPPRDFA AYRSKKVAKK PTNKAPHPKQ |
| | EPQEINFPDD LPGSNTAAPV QETLHGCQPV TQEDGKESRI SVQERQ |
| SEQ ID NO: 93 | |
| vector clone: | pIB1072 |
| Sequence: | MNRGVPFRHL LLVLQLALLP AATQGEQKLI SEEDLKKVVL GKKGDTVELT |
| | CTASQKKSIQ FHWKNSNQIK ILGNQGSFLT KGPSKLNDRA DSRRSLWDQG |
| | NFPLIIKNLK IEDSDTYICE VEDQKEEVQL LVFGLTANSD THLLQGQSLT |
| | LTLESPPGSS PSVQCRSPRG KNIQGGKTLS VSQLELQDSG TWTCTVLQNQ |
| | KKVEFKIDIV VLAFQKASSI VYKKEGEQVE FSFPLAFTVE KLTGSGELWW |
| | QAERASSSKS WITFDLKNKE VSVKRVTQDP KLQMGKKLPL HLTLPQALPQ |
| | YAGSGNLTLA LEAKTGKLHQ EVNLVVMRAT QLQKNLTCEV WGPTSPKLML |
| | SLKLENKEAK VSKREKAVWV LNPEAGMWQC LLSDSGQVLL ESNIKVLPTW |
| | STPVQPMALI VLGGVAGLLL FIGLGIFFCV RCRHRRRQAE RMSQIKRLLS |
| | EKKTCQCPHR FQKTCSPI |
| SEQ ID NO: 94 | |
| vector clone: | pIB1073 |
| Sequence: | MNRGVPFRHL LLVLQLALLP AATQGEQKLI SEEDLKKVVL GKKGDTVELT |
| | CTASQKKSIQ FHWKNSNQIK ILGNQGSFLT KGPSKLNDRA DSRRSLWDQG |
| | NFPLIIKNLK IEDSDTYICE VEDQKEEVQL LVFGLTANSD THLLQGQSLT |
| | LTLESPPGSS PSVQCRSPRG KNIQGGKTLS VSQLELQDSG TWTCTVLQNQ |
| | KKVEFKIDIV VLAFQKASSI VYKKEGEQVE FSFPLAFTVE KLTGSGELWW |
| | QAERASSSKS WITFDLKNKE VSVKRVTQDP KLQMGKKLPL HLTLPQALPQ |
| | YAGSGNLTLA LEAKTGKLHQ EVNLVVMRAT QLQKNLTCEV WGPTSPKLML |
| | SLKLENKEAK VSKREKAVWV LNPEAGMWQC LLSDSGQVLL ESNIKVLPTW |
| | STPVQPMALI VLGGVAGLLL FIGLGIFFCV RCRHRRRQAE RMSQIKRLLS |
| | EKKTCQCPHR FQKTCSPIRS KRSRLLHSDY MNMTPRRPGP TRKHYQPYAP |
| | PRDFAAYRSK KVAKKPTNKA PHPKQEPQEI NFPDDLPGSN TAAPVQETLH |
| | GCQPVTQEDG KESRISVQER Q |
| SEQ ID NO: 95 | |
| vector clone: | pIB1074 |
| Sequence: | MALPVTALLL PLALLLHAAR PEQKLISEED LSQFRVSPLD RTWNLGETVE |
| | LKCQVLLSNP TSGCSWLFQP RGAAASPTFL LYLSQNKPKA AEGLDTQRFS |
| | GKRLGDTFVL TLSDFRRENE GYYFCSALSN SIMYFSHFVP VFLPAKPTTT |
| | PAPRPPTPAP TIASQPLSLR PEACRPAAGG AVHTRGLDFA CDIYIWAPLA |
| | GTCGVLLLSL VITLYCNHRN RRRVCKCPRP VVKSGDKPSL SARYVRAKRG |
| | SGEGRGSLLT CGDVEENPGP MRPRLWLLLA AQLTVLHGNS VDYKDDDDKL |
| | QQTPAYIKVQ TNKMVMLSCE AKISLSNMRI YWLRQRQAPS SDSHHEFLAL |
| | WDSAKGTIHG EEVEQEKIAV FRDASRFILN LTSVKPEDSG IYFCMIVGSP |
| | ELTFGKGTQL SVVDFLPTTA QPTKKSTLKK RVCRLPRPET QKGPLCSPIT |
| | LGLLVAGVLV LLVSLGVAIH LCCRRRRARL RFMKQFYK |
| SEQ ID NO: 96 | |
| vector clone: | pIB1075 |
| Sequence: | MALPVTALLL PLALLLHAAR PEQKLISEED LSQFRVSPLD RTWNLGETVE |
| | LKCQVLLSNP TSGCSWLFQP RGAAASPTFL LYLSQNKPKA AEGLDTQRFS |
| | GKRLGDTFVL TLSDFRRENE GYYFCSALSN SIMYFSHFVP VFLPAKPTTT |
| | PAPRPPTPAP TIASQPLSLR PEACRPAAGG AVHTRGLDFA CDIYIWAPLA |
| | GTCGVLLLSL VITLYCNHRN RRRVCKCPRP VVKSGDKPSL SARYVRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQRA |
| | KRGSGEGRGS LLTCGDVEEN PGPMRPRLWL LLAAQLTVLH GNSVDYKDDD |
| | DKLQQTPAYI KVQTNKMVML SCEAKISLSN MRIYWLRQRQ APSSDSHHEF |
| | LALWDSAKGT IHGEEVEQEK IAVFRDASRF ILNLTSVKPE DSGIYFCMIV |
| | GSPELTFGKG TQLSVVDFLP TTAQPTKKST LKKRVCRLPR PETQKGPLCS |
| | PITLGLLVAG VLVLLVSLGV AIHLCCRRRR ARLRFMKQFY KRSKRSRLLH |
| | SDYMNMTPRR PGPTRKHYQP YAPPRDFAAY RSKKVAKKPT NKAPHPKQEP |
| | QEINFPDDLP GSNTAAPVQE TLHGCQPVTQ EDGKESRISV QERQ |
| SEQ ID NO: 97 | |
| vector clone: | pIB1076 |
| Sequence: | MALPVTALLL PLALLLHAAR PEQKLISEED LELTDTLQAE TDQLEDEKSA |
| | LQTEIANLLK EKEKLEFILA AHNCTYGCTG PGLEGCPTNG PKIPSIATGM |
| | VGALLLLLVV ALGIGLFMRS KRSRLLHSDY MNMTPRRPGP TRKHYQPYAP |
| | PRDFAAYRSK KVAKKPTNKA PHPKQEPQEI NFPDDLPGSN TAAPVQETLH |
| | GCQPVTQEDG KESRISVQER Q |
| SEQ ID NO: 98 | |
| vector clone: | pIB1077 |
| Sequence: | MALPVTALLL PLALLLHAAR PEQKLISEED LELTDTLQAE TDQLEDEKSA |
| | LQTEIANLLK EKEKLEFILA AHFWVLVVVG GVLACYSLLV TVAFIIFWVR |
| | SKRSRLLHSD YMNMTPRRPG PTRKHYQPYA PPRDFAAYRS KKVAKKPTNK |
| | APHPKQEPQE INFPDDLPGS NTAAPVQETL HGCQPVTQED GKESRISVQE |
| | RQ |
| SEQ ID NO: 99 | |
| vector clone: | pIB1078 |
| Sequence: | MALPVTALLL PLALLLHAAR PEQKLISEED LLEEKVKTLK AQNSELASTA |
| | NMLREQVAQL NCTYGCTGPG LEGCPTNGPK IPSIATGMVG ALLLLLVVAL |
| | GIGLFMRSKR SRLLHSDYMN MTPRRPGPTR KHYQPYAPPR DFAAYRSKKV |
| | AKKPTNKAPH PKQEPQEINF PDDLPGSNTA APVQETLHGC QPVTQEDGKE |
| | SRISVQERQ |
| SEQ ID | |
| NO: 100 | |
| vector clone: | pIB1079 |
| Sequence: | MALPVTALLL PLALLLHAAR PEQKLISEED LLEEKVKTLK AQNSELASTA |
| | NMLREQVAQL FWVLVVVGGV LACYSLLVTV AFIIFWVRSK RSRLLHSDYM |
| | NMTPRRPGPT RKHYQPYAPP RDFAAYRSKK VAKKPTNKAP HPKQEPQEIN |
| | FPDDLPGSNT AAPVQETLHG CQPVTQEDGK ESRISVQERQ |
| SEQ ID | |
| NO: 101 | |
| vector clone: | pIB1080 |
| Sequence: | MALPVTALLL PLALLLHAAR PEQKLISEED LETQHKVLEL TAENERLQKK |
| | VEQLSRELST NCTYGCTGPG LEGCPTNGPK IPSIATGMVG ALLLLLVVAL |
| | GIGLFMRSKR SRLLHSDYMN MTPRRPGPTR KHYQPYAPPR DFAAYRSKKV |
| | AKKPTNKAPH PKQEPQEINF PDDLPGSNTA APVQETLHGC QPVTQEDGKE |
| | SRISVQERQ |
| SEQ ID | |
| NO: 102 | |
| vector clone: | pIB1081 |
| Sequence: | MALPVTALLL PLALLLHAAR PEQKLISEED LETQHKVLEL TAENERLQKK |
| | VEQLSRELST FWVLVVVGGV LACYSLLVTV AFIIFWVRSK RSRLLHSDYM |
| | NMTPRRPGPT RKHYQPYAPP RDFAAYRSKK VAKKPTNKAP HPKQEPQEIN |
| | FPDDLPGSNT AAPVQETLHG CQPVTQEDGK ESRISVQERQ |
| SEQ ID | |
| NO: 103 | |
| vector clone: | pIB1082 |
| Sequence: | MRPSGTAGAA LLALLAALCP ASRAEQKLIS EEDLLEEKKV CQGTSNKLTQ |
| | LGTFEDHFLS LQRMENNCEV VLGNLEITYV QRNYDLSFLK TIQEVAGYVL |
| | IALNTVERIP LENLQIIRGN MYYENSYALA VLSNYDANKT GLKELPMRNL |
| | QEILHGAVRF SNNPALCNVE SIQWRDIVSS DFLSNMSMDF QNHLGSCQKC |
| | DPSCPNGSCW GAGEENCQKL TKIICAQQCS GRCRGKSPSD CCHNQCAAGC |
| | TGPRESDCLV CRKFRDEATC KDTCPPLMLY NPTTYQMDVN PEGKYSFGAT |
| | CVKKCPRNYV VTDHGSCVRA CGADSYEMEE DGVRKCKKCE GPCRKVCNGI |
| | GIGEFKDSLS INATNIKHFK NCTSISGDLH ILPVAFRGDS FTHTPPLDPQ |
| | ELDILKTVKE ITGFLLIQAW PENRTDLHAF ENLEIIRGRT KQHGQFSLAV |
| | VSLNITSLGL RSLKEISDGD VIISGNKNLC YANTINWKKL FGTSGQKTKI |
| | ISNRGENSCK ATGQVCHALC SPEGCWGPEP RDCVSCRNVS RGRECVDKCN |
| | LLEGEPREFV ENSECIQCHP ECLPQAMNIT CTGRGPDNCI QCAHYIDGPH |
| | CVKTCPAGVM GENNTLVWKY ADAGHVCHLC HPNCTYGCTG PGLEGCPTNG |
| | PKIPSIATGM VGALLLLLVV ALGIGLFMRS KRSRLLHSDY MNMTPRRPGP |
| | TRKHYQPYAP PRDFAAYRSK KVAKKPTNKA PHPKQEPQEI NFPDDLPGSN |
| | TAAPVQETLH GCQPVTQEDG KESRISVQER Q |
| SEQ ID | |
| NO: 104 | |
| vector clone: | pIB1083 |
| Sequence: | MRPSGTAGAA LLALLAALCP ASRAEQKLIS EEDLGTSGQK TKIISNRGEN |
| | SCKATGQVCH ALCSPEGCWG PEPRDCVSCR NVSRGRECVD KCNLLEGEPR |
| | EFVENSECIQ CHPECLPQAM NITCTGRGPD NCIQCAHYID GPHCVKTCPA |
| | GVMGENNTLV WKYADAGHVC HLCHPNCTYG CTGPGLEGCP TNGPKIPSIA |
| | TGMVGALLLL LVVALGIGLF MRSKRSRLLH SDYMNMTPRR PGPTRKHYQP |
| | YAPPRDFAAY RSKKVAKKPT NKAPHPKQEP QEINFPDDLP GSNTAAPVQE |
| | TLHGCQPVTQ EDGKESRISV QERQ |
| SEQ ID | |
| NO: 105 | |
| vector clone: | pIB1084 |
| Sequence: | MRPSGTAGAA LLALLAALCP ASRAEQKLIS EEDLNCTYGC TGPGLEGCPT |
| | NGPKIPSIAT GMVGALLLLL VVALGIGLFM RSKRSRLLHS DYMNMTPRRP |
| | GPTRKHYQPY APPRDFAAYR SKKVAKKPTN KAPHPKQEPQ EINFPDDLPG |
| | SNTAAPVQET LHGCQPVTQE DGKESRISVQ ERQ |
| SEQ ID | |
| NO: 106 | |
| vector clone: | pIB1085 |
| Sequence: | MELAALCRWG LLLALLPPGA ASEQKLISEE DLTQVCTGTD MKLRLPASPE |
| | THLDMLRHLY QGCQVVQGNL ELTYLPTNAS LSFLQDIQEV QGYVLIAHNQ |
| | VRQVPLQRLR IVRGTQLFED NYALAVLDNG DPLNNTTPVT GASPGGLREL |
| | QLRSLTEILK GGVLIQRNPQ LCYQDTILWK DIFHKNNQLA LTLIDTNRSR |
| | ACHPCSPMCK GSRCWGESSE DCQSLTRTVC AGGCARCKGP LPTDCCHEQC |
| | AAGCTGPKHS DCLACLHFNH SGICELHCPA LVTYNTDTFE SMPNPEGRYT |
| | FGASCVTACP YNYLSTDVGS CTLVCPLHNQ EVTAEDGTQR CEKCSKPCAR |
| | VCYGLGMEHL REVRAVTSAN IQEFAGCKKI FGSLAFLPES FDGDPASNTA |
| | PLQPEQLQVF ETLEEITGYL YISAWPDSLP DLSVFQNLQV IRGRILHNGA |
| | YSLTLQGLGI SWLGLRSLRE LGSGLALIHH NTHLCFVHTV PWDQLFRNPH |
| | QALLHTANRP EDECVGEGLA CHQLCARGHC WGPGPTQCVN CSQFLRGQEC |
| | VEECRVLQGL PREYVNARHC LPCHPECQPQ NGSVTCFGPE ADQCVACAHY |
| | KDPPFCVARC PSGVKPDLSY MPIWKFPDEE GACQPCPINC THSCVDLDDK |
| | GCPAEQRASP LTSIISAVVG ILLVVVLGVV FGILIRSKRS RLLHSDYMNM |
| | TPRRPGPTRK HYQPYAPPRD FAAYRSKKVA KKPTNKAPHP KQEPQEINFP |
| | DDLPGSNTAA PVQETLHGCQ PVTQEDGKES RISVQERQ |
| SEQ ID | |
| NO: 107 | |
| vector clone: | pIB1086 |
| Sequence: | MELAALCRWG LLLALLPPGA ASEQKLISEE DLTQVCTGTD MKLRLPASPE |
| | THLDMLRHLY QGCQVVQGNL ELTYLPTNAS LSFLQDIQEV QGYVLIAHNQ |
| | VRQVPLQRLR IVRGTQLFED NYALAVLDNG DPLNNTTPVT GASPGGLREL |
| | QLRSLTEILK GGVLIQRNPQ LCYQDTILWK DIFHKNNQLA LTLIDTNRSR |
| | ACHPCSPMCK GSRCWGESSE DCQSLTRTVC AGGCARCKGP LPTDCCHEQC |
| | AAGCTGPKHS DCLACLHFNH SGICELHCPA LVTYNTDTFE SMPNPEGRYT |
| | FGASCVTACP YNYLSTDVGS CTLVCPLHNQ EVTAEDGTQR CEKCSKPCAR |
| | VCYGLGMEHL REVRAVTSAN IQEFAGCKKI FGSLAFLPES FDGDPASNTA |
| | PLQPEQLQVF ETLEEITGYL YISAWPDSLP DLSVFQNLQV IRGRILHNGA |
| | YSLTLQGLGI SWLGLRSLRE LGSGLALIHH NTHLCFVHTV PWDQLFRNPH |
| | QALLHTANRP EDECVGEGLA CHQLCARGHC WGPGPTQCVN CSQFLRGQEC |
| | VEECRVLQGL PREYVNARHC LPCHPECQPQ NGSVTCFGPE ADQCVACAHY |
| | KDPPFCVARC PSGVKPDLSY MPIWKFPDEE GACQPCPINC THSCVDLDDK |
| | GCPAEQRASP LTSIISAVEG ILLVVVLGVV FGILIRSKRS RLLHSDYMNM |
| | TPRRPGPTRK HYQPYAPPRD FAAYRSKKVA KKPTNKAPHP KQEPQEINFP |
| | DDLPGSNTAA PVQETLHGCQ PVTQEDGKES RISVQERQ |
| SEQ ID | |
| NO: 108 | |
| vector clone: | pIB1087 |
| Sequence: | MELAALCRWG LLLALLPPGA ASEQKLISEE DLTQVCTGTD MKLRLPASPE |
| | THLDMLRHLY QGCQVVQGNL ELTYLPTNAS LSFLQDIQEV QGYVLIAHNQ |
| | VRQVPLQRLR IVRGTQLFED NYALAVLDNG DPLNNTTPVT GASPGGLREL |
| | QLRSLTEILK GGVLIQRNPQ LCYQDTILWK DIFHKNNQLA LTLIDTNRSR |
| | ACHPCSPMCK GSRCWGESSE DCQSLTRTVC AGGCARCKGP LPTDCCHEQC |
| | AAGCTGPKHS DCLACLHFNH SGICELHCPA LVTYNTDTFE SMPNPEGRYT |
| | FGASCVTACP YNYLSTDVGS CTLVCPLHNQ EVTAEDGTQR CEKCSKPCAR |
| | VCYGLGMEHL REVRAVTSAN IQEFAGCKKI FGSLAFLPES FDGDPASNTA |
| | PLQPEQLQVF ETLEEITGYL YISAWPDSLP DLSVFQNLQV IRGRILHNGA |
| | YSLTLQGLGI SWLGLRSLRE LGSGLALIHH NTHLCFVHTV PWDQLFRNPH |
| | QALLHTANRP EDECVGEGLA CHQLCARGHC WGPGPTQCVN CSQFLRGQEC |
| | VEECRVLQGL PREYVNARHC LPCHPECQPQ NGSVTCFGPE ADQCVACAHY |
| | KDPPFCVARC PSGVKPDLSY MPIWKFPDEE GACQPCPINC THSCVDLDDK |
| | GCPAEQRASP LTSIISAVVD ILLVVVLGVV FGILIRSKRS RLLHSDYMNM |
| | TPRRPGPTRK HYQPYAPPRD FAAYRSKKVA KKPTNKAPHP KQEPQEINFP |
| | DDLPGSNTAA PVQETLHGCQ PVTQEDGKES RISVQERQ |
| SEQ ID | |
| NO: 109 | |
| vector clone: | pIB1088 |
| Sequence: | MELAALCRWG LLLALLPPGA ASEQKLISEE DLTQVCTGTD MKLRLPASPE |
| | THLDMLRHLY QGCQVVQGNL ELTYLPTNAS LSFLQDIQEV QGYVLIAHNQ |
| | VRQVPLQRLR IVRGTQLFED NYALAVLDNG DPLNNTTPVT GASPGGLREL |
| | QLRSLTEILK GGVLIQRNPQ LCYQDTILWK DIFHKNNQLA LTLIDTNRSR |
| | ACHPCSPMCK GSRCWGESSE DCQSLTRTVC AGGCARCKGP LPTDCCHEQC |
| | AAGCTGPKHS DCLACLHFNH SGICELHCPA LVTYNTDTFE SMPNPEGRYT |
| | FGASCVTACP YNYLSTDVGS CTLVCPLHNQ EVTAEDGTQR CEKCSKPCAR |
| | VCYGLGMEHL REVRAVTSAN IQEFAGCKKI FGSLAFLPES FDGDPASNTA |
| | PLQPEQLQVF ETLEEITGYL YISAWPDSLP DLSVFQNLQV IRGRILHNGA |
| | YSLTLQGLGI SWLGLRSLRE LGSGLALIHH NTHLCFVHTV PWDQLFRNPH |
| | QALLHTANRP EDECVGEGLA CHQLCARGHC WGPGPTQCVN CSQFLRGQEC |
| | VEECRVLQGL PREYVNARHC LPCHPECQPQ NGSVTCFGPE ADQCVACAHY |
| | KDPPFCVARC PSGVKPDLSY MPIWKFPDEE GACQPCPINC THSCVDLDDK |
| | GCPAEQRASP LTSIISAVVR ILLVVVLGVV FGILIRSKRS RLLHSDYMNM |
| | TPRRPGPTRK HYQPYAPPRD FAAYRSKKVA KKPTNKAPHP KQEPQEINFP |
| | DDLPGSNTAA PVQETLHGCQ PVTQEDGKES RISVQERQ |
| SEQ ID | |
| NO: 110 | |
| vector clone: | pIB1089 |
| Sequence: | MELAALCRWG LLLALLPPGA ASEQKLISEE DLQLCARGHC WGPGPTQCVN |
| | CSQFLRGQEC VEECRVLQGL PREYVNARHC LPCHPECQPQ NGSVTCFGPE |
| | ADQCVACAHY KDPPFCVARC PSGVKPDLSY MPIWKFPDEE GACQPCPINC |
| | THSCVDLDDK GCPAEQRASP LTSIISAVVG ILLVVVLGVV FGILIRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 111 | |
| vector clone: | pIB1090 |
| Sequence: | MELAALCRWG LLLALLPPGA ASEQKLISEE DLQLCARGHC WGPGPTQCVN |
| | CSQFLRGQEC VEECRVLQGL PREYVNARHC LPCHPECQPQ NGSVTCFGPE |
| | ADQCVACAHY KDPPFCVARC PSGVKPDLSY MPIWKFPDEE GACQPCPINC |
| | THSCVDLDDK GCPAEQRASP LTSIISAVEG ILLVVVLGVV FGILIRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 112 | |
| vector clone: | pIB1091 |
| Sequence: | MELAALCRWG LLLALLPPGA ASEQKLISEE DLQLCARGHC WGPGPTQCVN |
| | CSQFLRGQEC VEECRVLQGL PREYVNARHC LPCHPECQPQ NGSVTCFGPE |
| | ADQCVACAHY KDPPFCVARC PSGVKPDLSY MPIWKFPDEE GACQPCPINC |
| | THSCVDLDDK GCPAEQRASP LTSIISAVVD ILLVVVLGVV FGILIRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 113 | |
| vector clone: | pIB1092 |
| Sequence: | MELAALCRWG LLLALLPPGA ASEQKLISEE DLQLCARGHC WGPGPTQCVN |
| | CSQFLRGQEC VEECRVLQGL PREYVNARHC LPCHPECQPQ NGSVTCFGPE |
| | ADQCVACAHY KDPPFCVARC PSGVKPDLSY MPIWKFPDEE GACQPCPINC |
| | THSCVDLDDK GCPAEQRASP LTSIISAVVR ILLVVVLGVV FGILIRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 114 | |
| vector clone: | pIB1093 |
| Sequence: | MELAALCRWG LLLALLPPGA ASEQKLISEE DLSIISAVVG ILLVVVLGVV |
| | FGILIRSKRS RLLHSDYMNM TPRRPGPTRK HYQPYAPPRD FAAYRSKKVA |
| | KKPTNKAPHP KQEPQEINFP DDLPGSNTAA PVQETLHGCQ PVTQEDGKES |
| | RISVQERQ |
| SEQ ID | |
| NO: 115 | |
| vector clone: | pIB1094 |
| Sequence: | MELAALCRWG LLLALLPPGA ASEQKLISEE DLSIISAVEG ILLVVVLGVV |
| | FGILIRSKRS RLLHSDYMNM TPRRPGPTRK HYQPYAPPRD FAAYRSKKVA |
| | KKPTNKAPHP KQEPQEINFP DDLPGSNTAA PVQETLHGCQ PVTQEDGKES |
| | RISVQERQ |
| SEQ ID | |
| NO: 116 | |
| vector clone: | pIB1095 |
| Sequence: | MELAALCRWG LLLALLPPGA ASEQKLISEE DLSIISAVVD ILLVVVLGVV |
| | FGILIRSKRS RLLHSDYMNM TPRRPGPTRK HYQPYAPPRD FAAYRSKKVA |
| | KKPTNKAPHP KQEPQEINFP DDLPGSNTAA PVQETLHGCQ PVTQEDGKES |
| | RISVQERQ |
| SEQ ID | |
| NO: 117 | |
| vector clone: | pIB1096 |
| Sequence: | MELAALCRWG LLLALLPPGA ASEQKLISEE DLSIISAVVR ILLVVVLGVV |
| | FGILIRSKRS RLLHSDYMNM TPRRPGPTRK HYQPYAPPRD FAAYRSKKVA |
| | KKPTNKAPHP KQEPQEINFP DDLPGSNTAA PVQETLHGCQ PVTQEDGKES |
| | RISVQERQ |
| SEQ ID | |
| NO: 118 | |
| vector clone: | pIB1097 |
| Sequence: | MALPVTALLL PLALLLHAAR PEQKLISEED LQEQLVESGG RLVTPGTPLT |
| | LTCTASGFSL GSDFMSWVRQ APGKGLEWIG YIDPRSDIPY YASWAKGRFT |
| | ISKTSTTVDL KITSPTTEDT ATYFCARDLN AGYFNGIFYI WGPGTLVTVS |
| | SGGGGSGGGG SGGGGSELVM TQTPSSVSAA VGDTVTINCQ ASETVATLLA |
| | WYQQKPGQPP KLLIYGASNL ESGVPSRFRG SGSGTEFTLT ISGMKAEDAA |
| | TYYCQYGYIS TGSNTFGAGT NVEIKAAAGS GGSGILVKQS PMLVAYDNAV |
| | NLSCKYSYNL FSREFRASLH KGLDSAVEVC VVYGNYSQQL QVYSKTGFNC |
| | DGKLGNESVT FYLQNLYVNQ TDIYFCKIEV MYPPPYLDNE KSNGTIIHVK |
| | GKHLCPSPLF PGPSKPFWVL VVVGGVLACY SLLVTVAFII FWVRSKRSRL |
| | LHSDYMNMTP RRPGPTRKHY QPYAPPRDFA AYRSKKVAKK PTNKAPHPKQ |
| | EPQEINFPDD LPGSNTAAPV QETLHGCQPV TQEDGKESRI SVQERQ |
| SEQ ID | |
| NO: 119 | |
| vector clone: | pIB1098 |
| Sequence: | MALPVTALLL PLALLLHAAR PEQKLISEED LELVMTQTPS SVSAAVGDTV |
| | TINCQASETV ATLLAWYQQK PGQPPKLLIY GASNLESGVP SRFRGSGSGT |
| | EFTLTISGMK AEDAATYYCQ YGYISTGSNT FGAGTNVEIK GGGGSGGGGS |
| | GGGGSQEQLV ESGGRLVTPG TPLTLTCTAS GFSLGSDFMS WVRQAPGKGL |
| | EWIGYIDPRS DIPYYASWAK GRFTISKTST TVDLKITSPT TEDTATYFCA |
| | RDLNAGYFNG IFYIWGPGTL VTVSSAAAGS GGSGILVKQS PMLVAYDNAV |
| | NLSCKYSYNL FSREFRASLH KGLDSAVEVC VVYGNYSQQL QVYSKTGFNC |
| | DGKLGNESVT FYLQNLYVNQ TDIYFCKIEV MYPPPYLDNE KSNGTIIHVK |
| | GKHLCPSPLF PGPSKPFWVL VVVGGVLACY SLLVTVAFII FWVRSKRSRL |
| | LHSDYMNMTP RRPGPTRKHY QPYAPPRDFA AYRSKKVAKK PTNKAPHPKQ |
| | EPQEINFPDD LPGSNTAAPV QETLHGCQPV TQEDGKESRI SVQERQ |
| SEQ ID | |
| NO: 120 | |
| vector clone: | pIB1099 |
| Sequence: | MALPVTALLL PLALLLHAAR PEQKLISEED LQEQLVESGG RLVTPGTPLT |
| | LTCTASGFSL GSDEMSWVRQ APGKGLEWIG YIDPRSDIPY YASWAKGRFT |
| | ISKTSTTVDL KITSPTTEDT ATYFCARDLN AGYFNGIFYI WGPGTLVTVS |
| | SGGGGSGGGG SGGGGSELDM TQTPSSTSEP VGGTVTINCQ ASQTISSYLS |
| | WYQQKPGHPP KLLIYDASDL ASGVPSRFSG SRSGTQFTLT ISGVQCDDAA |
| | TYYCLGVYDY RSDDGAAFGG GTELEILAAA GSGGSGILVK QSPMLVAYDN |
| | AVNLSCKYSY NLFSREFRAS LHKGLDSAVE VCVVYGNYSQ QLQVYSKTGF |
| | NCDGKLGNES VTFYLQNLYV NQTDIYFCKI EVMYPPPYLD NEKSNGTIIH |
| | VKGKHLCPSP LFPGPSKPFW VLVVVGGVLA CYSLLVTVAF IIFWVRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 121 | |
| vector clone: | pIB1100 |
| Sequence: | MALPVTALLL PLALLLHAAR PEQKLISEED LELDMTQTPS STSEPVGGTV |
| | TINCQASQTI SSYLSWYQQK PGHPPKLLIY DASDLASGVP SRFSGSRSGT |
| | QFTLTISGVQ CDDAATYYCL GVYDYRSDDG AAFGGGTELE ILGGGGSGGG |
| | GSGGGGSQEQ LVESGGRLVT PGTPLTLTCT ASGFSLGSDF MSWVRQAPGK |
| | GLEWIGYIDP RSDIPYYASW AKGRFTISKT STTVDLKITS PTTEDTATYF |
| | CARDLNAGYF NGIFYIWGPG TLVTVSSAAA GSGGSGILVK QSPMLVAYDN |
| | AVNLSCKYSY NLFSREFRAS LHKGLDSAVE VCVVYGNYSQ QLQVYSKTGF |
| | NCDGKLGNES VTFYLQNLYV NQTDIYFCKI EVMYPPPYLD NEKSNGTIIH |
| | VKGKHLCPSP LFPGPSKPFW VLVVVGGVLA CYSLLVTVAF IIFWVRSKRS |
| | RLLHSDYMNM TPRRPGPTRK HYQPYAPPRD FAAYRSKKVA KKPTNKAPHP |
| | KQEPQEINFP DDLPGSNTAA PVQETLHGCQ PVTQEDGKES RISVQERQ |
| SEQ ID | |
| NO: 122 | |
| vector clone: | pIB1101 |
| Sequence: | MALPVTALLL PLALLLHAAR PEQKLISEED LQSLEESGGR LVTPGTPLTL |
| | TCTVSGFSLS TNDMNWVRQA PGKGLEWIGV IYSDDTPDYA TWAKGRFTIS |
| | RTSTTVDLKI TSPTTEDTAT YFCARGHYDS AVYAYALNIW GPGTLVTVSS |
| | GGGGSGGGGS GGGGSELVMT QTPSSVSAAV GGTVTITCQA SQSLSNLLAW |
| | YQQKPGQPPK LLIYGASNLE SGVPSRFRGS GSGTDFTLTI SGMKAEDAAT |
| | YYCQGGHYSG LTFGNGTNVE IKAAAGSGGS GILVKQSPML VAYDNAVNLS |
| | CKYSYNLESR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID | |
| NO: 123 | |
| vector clone: | pIB1102 |
| Sequence: | MALPVTALLL PLALLLHAAR PEQKLISEED LELVMTQTPS SVSAAVGGTV |
| | TITCQASQSL SNLLAWYQQK PGQPPKLLIY GASNLESGVP SRFRGSGSGT |
| | DFTLTISGMK AEDAATYYCQ GGHYSGLTFG NGTNVEIKGG GGSGGGGSGG |
| | GGSQSLEESG GRLVTPGTPL TLTCTVSGFS LSTNDMNWVR QAPGKGLEWI |
| | GVIYSDDTPD YATWAKGRFT ISRTSTTVDL KITSPTTEDT ATYFCARGHY |
| | DSAVYAYALN IWGPGTLVTV SSAAAGSGGS GILVKQSPML VAYDNAVNLS |
| | CKYSYNLFSR EFRASLHKGL DSAVEVCVVY GNYSQQLQVY SKTGFNCDGK |
| | LGNESVTFYL QNLYVNQTDI YFCKIEVMYP PPYLDNEKSN GTIIHVKGKH |
| | LCPSPLFPGP SKPFWVLVVV GGVLACYSLL VTVAFIIFWV RSKRSRLLHS |
| | DYMNMTPRRP GPTRKHYQPY APPRDFAAYR SKKVAKKPTN KAPHPKQEPQ |
| | EINFPDDLPG SNTAAPVQET LHGCQPVTQE DGKESRISVQ ERQ |
| SEQ ID | |
| NO: 124 | |
| vector clone: | pIB1103 |
| Sequence: | MYGKIIFVLL LSEIVSISAE QKLISEEDLS STTGVAMHTS TSSSVTKSYI |
| | SSQTNDTHKR DTYAATPRAH EVSEISVRTV YPPEEETGER VQLAHHFSEP |
| | EITLIIFGVM AGVIGTILLI SYGRSKRSRL LHSDYMNMTP RRPGPTRKHY |
| | QPYAPPRDFA AYRSKKVAKK PTNKAPHPKQ EPQEINFPDD LPGSNTAAPV |
| | QETLHGCQPV TQEDGKESRI SVQERQ |
| SEQ ID | |
| NO: 125 | |
| vector clone: | pIB1104 |
| Sequence: | MYGKIIFVLL LSEIVSISAE QKLISEEDLI TLIIFGVMAG VIGTILLISY |
| | GRSKRSRLLH SDYMNMTPRR PGPTRKHYQP YAPPRDFAAY RSKKVAKKPT |
| | NKAPHPKQEP QEINFPDDLP GSNTAAPVQE TLHGCQPVTQ EDGKESRISV |
| | QERQ |
| SEQ ID | |
| NO: 126 | |
| vector clone: | pIB1105 |
| Sequence: | MDHLGASLWP QVGSLCLLLA GAAWEQKLIS EEDLAPPPNL PDPKFESKAA |
| | LLAARGPEEL LCFTERLEDL VCFWEEAASA GVGPGNYSFS YQLEDEPWKL |
| | CRLHQAPTAR GAVRFWCSLP TADTSSFVPL ELRVTAASGA PRYHRVIHIN |
| | EVVLLDAPVG LVARLADESG HVVLRWLPPP ETPMTSHIRY EVDVSAGNGA |
| | GSVQRVEILE GRTECVLSNL RGRTRYTFAV RARMAEPSFG GFWSAWSEPV |
| | SLLTPSDLDP LILTLSLILV VILVLLTVLA LLSRSKRSRL LHSDYMNMTP |
| | RRPGPTRKHY QPYAPPRDFA AYRSKKVAKK PTNKAPHPKQ EPQEINFPDD |
| | LPGSNTAAPV QETLHGCQPV TQEDGKESRI SVQERQ |
| SEQ ID | |
| NO: 127 | |
| vector clone: | pIB1106 |
| Sequence: | MDHLGASLWP QVGSLCLLLA GAAWEQKLIS EEDLLILTLS LILVVILVLL |
| | TVLALLSRSK RSRLLHSDYM NMTPRRPGPT RKHYQPYAPP RDFAAYRSKK |
| | VAKKPTNKAP HPKQEPQEIN FPDDLPGSNT AAPVQETLHG CQPVTQEDGK |
| | ESRISVQERQ |
| SEQ ID | |
| NO: 128 | |
| vector clone: | pIB1107 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLSAVLGL LLLRWRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 129 | |
| vector clone: | pIB1108 |
| Sequence: | MPSWALFMVT SCLLLAPQNL AQVSSEQKLI SEEDLISLVT ALHLVLGLSA |
| | VLGLLLLRWR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 130 | |
| vector clone: | pIB1109 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLNAVLGL LLLRWRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 131 | |
| vector clone: | pIB1110 |
| Sequence: | MPSWALFMVT SCLLLAPQNL AQVSSEQKLI SEEDLISLVT ALHLVLGLNA |
| | VLGLLLLRWR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 132 | |
| vector clone: | pIB1111 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLSAVLGL LLLRKRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 133 | |
| vector clone: | pIB1112 |
| Sequence: | MPSWALFMVT SCLLLAPQNL AQVSSEQKLI SEEDLISLVT ALHLVLGLSA |
| | VLGLLLLRKR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 134 | |
| vector clone: | pIB1113 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLSAVLGL LLLRKRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 135 | |
| vector clone: | pIB1114 |
| Sequence: | MPSWALFMVT SCLLLAPQNL AQVSSEQKLI SEEDLISLVT ALLLVLGLSA |
| | VLGLLLLRWR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 136 | |
| vector clone: | pIB1115 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLNAVLGL LLLRKRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 137 | |
| vector clone: | pIB1116 |
| Sequence: | MPSWALFMVT SCLLLAPQNL AQVSSEQKLI SEEDLISLVT ALHLVLGLNA |
| | VLGLLLLRKR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 138 | |
| vector clone: | pIB1117 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLNAVLGL LLLRKRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 139 | |
| vector clone: | pIB1118 |
| Sequence: | MPSWALFMVT SCLLLAPQNL AQVSSEQKLI SEEDLISLVT ALYLVLGLNA |
| | VLGLLLLRWR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 140 | |
| vector clone: | pIB1119 |
| Sequence: | MPSWALFMVT SCLLLAPQNL AQVSSEQKLI SEEDLQDVSL LASDSEPLKC |
| | FSRTFEDLTC FWDEEEAAPS GTYQLLYAYP REKPRACPLS SQSMPHFGTR |
| | YVCQFPDQEE VRLFFPLHLW VKNVFLNQTR TQRVLFVDSV GLPAPPSIIK |
| | AMGGSQPGEL QISWEEPAPE ISDFLRYELR YGPRDPKNST GPTVIQLIAT |
| | ETCCPALQRP HSASALDQSP CAQPTMPWQD GPKQTSPSRE ASALTAEGGS |
| | CLISGLQPGN SYWLQLRSEP DGISLGGSWG SWSLPVTVDL PGDAVALGLQ |
| | CFTLDLKNVT CQWQQQDHAS SQGFFYHSRA RCCPRDRYPI WENCEEEEKT |
| | NPGLQTPQFS RCHFKSRNDS IIHILVEVTT APGTVHSYLG SPFWIHQAVR |
| | LPTPNLHWRE ISSGHLELEW QHPSSWAAQE TCYQLRYTGE GHQDWKVLEP |
| | PLGARGGTLE LRPRSRYRLQ LRARLNGPTY QGPWSSWSDP TRVETATETA |
| | WISLVTALHL VLGLSAVLGL LLLRKRSKRS RLLHSDYMNM TPRRPGPTRK |
| | HYQPYAPPRD FAAYRSKKVA KKPTNKAPHP KQEPQEINFP DDLPGSNTAA |
| | PVQETLHGCQ PVTQEDGKES RISVQERQ |
| SEQ ID | |
| NO: 141 | |
| vector clone: | pIB1120 |
| Sequence: | MPSWALFMVT SCLLLAPQNL AQVSSEQKLI SEEDLISLVT AWCLVLGLSA |
| | VLGLLLLRWR SKRSRLLHSD YMNMTPRRPG PTRKHYQPYA PPRDFAAYRS |
| | KKVAKKPTNK APHPKQEPQE INFPDDLPGS NTAAPVQETL HGCQPVTQED |
| | GKESRISVQE RQ |
| SEQ ID | |
| NO: 142 | |
| vector clone: | pIB1179 |
| Sequence: | MALPVTALLL PLALLLHAAR PEPKSCDKTH TCPPCPAPEL LGGPSVFLFP |
| | PKPKDTLMIS RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR |
| | EPQVYTLPPS RDELTKNQVS LTCLVKGFYP SDIAVEWESN GQPENNYKTT |
| | PPVLDSDGSF FLYSKLTVDK SRWQQGNVFS CSVMHEALHN HYTQKSLSLS |
| | PGKAAAFWVL VVVGGVLACY SLLVTVAFII FWVRSKRSRL LHSDYMNMTP |
| | RRPGPTRKHY QPYAPPRDFA AYRSKKVAKK PTNKAPHPKQ EPQEINFPDD |
| | LPGSNTAAPV QETLHGCQPV TQEDGKESRI SVQERQ |
| SEQ ID | |
| NO: 143 | |
| vector clone: | pIB1180 |
| Sequence: | MALPVTALLL PLALLLHAAR PAEPKSPDKT HTCPPCPAPP VAGPSVFLFP |
| | PKPKDTLMIA RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR |
| | EPQVYTLPPS RDELTKNQVS LTCLVKGFYP SDIAVEWESN GQPENNYKTT |
| | PPVLDSDGSF FLYSKLTVDK SRWQQGNVFS CSVMHEALHN HYTQKSLSLS |
| | PGKKDPKFWV LVVVGGVLAC YSLLVTVAFI IFWVRSKRSR LLHSDYMNMT |
| | PRRPGPTRKH YQPYAPPRDF AAYRSKKVAK KPTNKAPHPK QEPQEINFPD |
| | DLPGSNTAAP VQETLHGCQP VTQEDGKESR ISVQERQ |
| SEQ ID | |
| NO: 144 | |
| vector clone: | pIB1181 |
| Sequence: | MALPVTALLL PLALLLHAAR PERKCCVECP PCPAPPVAGP SVFLFPPKPK |
| | DTLMISRTPE VTCVVVDVSH EDPEVQFNWY VDGVEVHNAK TKPREEQFNS |
| | TFRVVSVLTV VHQDWINGKE YKCKVSNKGL PAPIEKTISK TKGQPREPQV |
| | YTLPPSREEM TKNQVSLTCL VKGFYPSDIS VEWESNGQPE NNYKTTPPML |
| | DSDGSFFLYS KLTVDKSRWQ QGNVFSCSVM HEALHNHYTQ KSLSLSPGKF |
| | WVLVVVGGVL ACYSLLVTVA FIIFWVRSKR SRLLHSDYMN MTPRRPGPTR |
| | KHYQPYAPPR DFAAYRSKKV AKKPTNKAPH PKQEPQEINF PDDLPGSNTA |
| | APVQETLHGC QPVTQEDGKE SRISVQERQ |
| SEQ ID | |
| NO: 145 | |
| vector clone: | pIB1182 |
| Sequence: | MALPVTALLL PLALLLHAAR PELKTPLGDT THTCPRCPEP KSCDTPPPCP |
| | RCPEPKSCDT PPPCPRCPEP KSCDTPPPCP RCPAPELLGG PSVFLFPPKP |
| | KDTLMISRTP EVTCVVVDVS HEDPEVQFKW YVDGVEVHNA KTKPREEQYN |
| | STFRVVSVLT VLHQDWLNGK EYKCKVSNKA LPAPIEKTIS KTKGQPREPQ |
| | VYTLPPSREE MTKNQVSLTC LVKGFYPSDI AVEWESSGQP ENNYNTTPPM |
| | LDSDGSFFLY SKLTVDKSRW QQGNIFSCSV MHEALHNRFT QKSLSLSPGK |
| | FWVLVVVGGV LACYSLLVTV AFIIFWVRSK RSRLLHSDYM NMTPRRPGPT |
| | RKHYQPYAPP RDFAAYRSKK VAKKPTNKAP HPKQEPQEIN FPDDLPGSNT |
| | AAPVQETLHG CQPVTQEDGK ESRISVQERQ |
| SEQ ID | |
| NO: 146 | |
| vector clone: | pIB1183 |
| Sequence: | MALPVTALLL PLALLLHAAR PESKYGPPCP SCPAPEFLGG PSVFLFPPKP |
| | KDTLMISRTP EVTCVVVDVS QEDPEVQFNW YVDGVEVHNA KTKPREEQFN |
| | STYRVVSVLT VLHQDWLNGK EYKCKVSNKG LPSSIEKTIS KAKGQPREPQ |
| | VYTLPPSQEE MTKNQVSLTC LVKGFYPSDI AVEWESNGQP ENNYKTTPPV |
| | LDSDGSFFLY SRLTVDKSRW QEGNVFSCSV MHEALHNHYT QKSLSLSLGK |
| | FWVLVVVGGV LACYSLLVTV AFIIFWVRSK RSRLLHSDYM NMTPRRPGPT |
| | RKHYQPYAPP RDFAAYRSKK VAKKPTNKAP HPKQEPQEIN FPDDLPGSNT |
| | AAPVQETLHG CQPVTQEDGK ESRISVQERQ |
| SEQ ID | |
| NO: 147 | |
| vector clone: | pIB1184 |
| Sequence: | MALPVTALLL PLALLLHAAR PESKYGPPCP PCPAPEFEGG PSVFLFPPKP |
| | KDTLMISRTP EVTCVVVDVS QEDPEVQFNW YVDGVEVHNA KTKPREEQFQ |
| | STYRVVSVLT VLHQDWLNGK EYKCKVSNKG LPSSIEKTIS KAKGQPREPQ |
| | VYTLPPSQEE MTKNQVSLTC LVKGFYPSDI AVEWESNGQP ENNYKTTPPV |
| | LDSDGSFFLY SRLTVDKSRW QEGNVFSCSV MHEALHNHYT QKSLSLSLGK |
| | FWVLVVVGGV LACYSLLVTV AFIIFWVRSK RSRLLHSDYM NMTPRRPGPT |
| | RKHYQPYAPP RDFAAYRSKK VAKKPTNKAP HPKQEPQEIN FPDDLPGSNT |
| | AAPVQETLHG CQPVTQEDGK ESRISVQERQ |
| SEQ ID | |
| NO: 148 | |
| vector clone: | pIB1185 |
| Sequence: | MALPVTALLL PLALLLHAAR PEPKSCDKTH TCPPCPAPEL LGGPSVFLFP |
| | PKPKDTLMIS RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR |
| | EPQVYTLPPS RDELTKNQVS LTCLVKGFYP SDIAVEWESN GQPENNYKTT |
| | PPVLDSDGSF FLYSKLTVDK SRWQQGNVFS CSVMHEALHN HYTQKSLSLS |
| | PGKAAAIEVM YPPPYLDNEK SNGTIIHVKG KHLCPSPLFP GPSKPFWVLV |
| | VVGGVLACYS LLVTVAFIIF WVRSKRSRLL HSDYMNMTPR RPGPTRKHYQ |
| | PYAPPRDFAA YRSKKVAKKP TNKAPHPKQE PQEINFPDDL PGSNTAAPVQ |
| | ETLHGCQPVT QEDGKESRIS VQERQ |
| SEQ ID | |
| NO: 149 | |
| vector clone: | pIB1186 |
| Sequence: | MALPVTALLL PLALLLHAAR PAEPKSPDKT HTCPPCPAPP VAGPSVFLFP |
| | PKPKDTLMIA RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR |
| | EPQVYTLPPS RDELTKNQVS LTCLVKGFYP SDIAVEWESN GQPENNYKTT |
| | PPVLDSDGSF FLYSKLTVDK SRWQQGNVFS CSVMHEALHN HYTQKSLSLS |
| | PGKKDPKIEV MYPPPYLDNE KSNGTIIHVK GKHLCPSPLF PGPSKPFWVL |
| | VVVGGVLACY SLLVTVAFII FWVRSKRSRL LHSDYMNMTP RRPGPTRKHY |
| | QPYAPPRDFA AYRSKKVAKK PTNKAPHPKQ EPQEINFPDD LPGSNTAAPV |
| | QETLHGCQPV TQEDGKESRI SVQERQ |
| SEQ ID | |
| NO: 150 | |
| vector clone: | pIB1187 |
| Sequence: | MALPVTALLL PLALLLHAAR PERKCCVECP PCPAPPVAGP SVFLFPPKPK |
| | DTLMISRTPE VTCVVVDVSH EDPEVQFNWY VDGVEVHNAK TKPREEQFNS |
| | TFRVVSVLTV VHQDWLNGKE YKCKVSNKGL PAPIEKTISK TKGQPREPQV |
| | YTLPPSREEM TKNQVSLTCL VKGFYPSDIS VEWESNGQPE NNYKTTPPML |
| | DSDGSFFLYS KLTVDKSRWQ QGNVFSCSVM HEALHNHYTQ KSLSLSPGKI |
| | EVMYPPPYLD NEKSNGTIIH VKGKHLCPSP LFPGPSKPFW VLVVVGGVLA |
| | CYSLLVTVAF IIFWVRSKRS RLLHSDYMNM TPRRPGPTRK HYQPYAPPRD |
| | FAAYRSKKVA KKPTNKAPHP KQEPQEINFP DDLPGSNTAA PVQETLHGCQ |
| | PVTQEDGKES RISVQERQ |
| SEQ ID | |
| NO: 151 | |
| vector clone: | pIB1188 |
| Sequence: | MALPVTALLL PLALLLHAAR PELKTPLGDT THTCPRCPEP KSCDTPPPCP |
| | RCPEPKSCDT PPPCPRCPEP KSCDTPPPCP RCPAPELLGG PSVFLFPPKP |
| | KDTLMISRTP EVTCVVVDVS HEDPEVQFKW YVDGVEVHNA KTKPREEQYN |
| | STFRVVSVLT VLHQDWLNGK EYKCKVSNKA LPAPIEKTIS KTKGQPREPQ |
| | VYTLPPSREE MTKNQVSLTC LVKGFYPSDI AVEWESSGQP ENNYNTTPPM |
| | LDSDGSFFLY SKLTVDKSRW QQGNIFSCSV MHEALHNRFT QKSLSLSPGK |
| | IEVMYPPPYL DNEKSNGTII HVKGKHLCPS PLFPGPSKPF WVLVVVGGVL |
| | ACYSLLVTVA FIIFWVRSKR SRLLHSDYMN MTPRRPGPTR KHYQPYAPPR |
| | DFAAYRSKKV AKKPTNKAPH PKQEPQEINF PDDLPGSNTA APVQETLHGC |
| | QPVTQEDGKE SRISVQERQ |
| SEQ ID | |
| NO: 152 | |
| vector clone: | pIB1189 |
| Sequence: | MALPVTALLL PLALLLHAAR PESKYGPPCP SCPAPEFLGG PSVFLFPPKP |
| | KDTLMISRTP EVTCVVVDVS QEDPEVQFNW YVDGVEVHNA KTKPREEQFN |
| | STYRVVSVLT VLHQDWLNGK EYKCKVSNKG LPSSIEKTIS KAKGQPREPQ |
| | VYTLPPSQEE MTKNQVSLTC LVKGFYPSDI AVEWESNGQP ENNYKTTPPV |
| | LDSDGSFFLY SRLTVDKSRW QEGNVFSCSV MHEALHNHYT QKSLSLSLGK |
| | IEVMYPPPYL DNEKSNGTII HVKGKHLCPS PLFPGPSKPF WVLVVVGGVL |
| | ACYSLLVTVA FIIFWVRSKR SRLLHSDYMN MTPRRPGPTR KHYQPYAPPR |
| | DFAAYRSKKV AKKPTNKAPH PKQEPQEINF PDDLPGSNTA APVQETLHGC |
| | QPVTQEDGKE SRISVQERQ |
| SEQ ID | |
| NO: 153 | |
| vector clone: | pIB1190 |
| Sequence: | MALPVTALLL PLALLLHAAR PESKYGPPCP PCPAPEFEGG PSVFLFPPKP |
| | KDTLMISRTP EVTCVVVDVS QEDPEVQFNW YVDGVEVHNA KTKPREEQFQ |
| | STYRVVSVLT VLHQDWLNGK EYKCKVSNKG LPSSIEKTIS KAKGQPREPQ |
| | VYTLPPSQEE MTKNQVSLTC LVKGFYPSDI AVEWESNGQP ENNYKTTPPV |
| | LDSDGSFFLY SRLTVDKSRW QEGNVFSCSV MHEALHNHYT QKSLSLSLGK |
| | IEVMYPPPYL DNEKSNGTII HVKGKHLCPS PLFPGPSKPF WVLVVVGGVL |
| | ACYSLLVTVA FIIFWVRSKR SRLLHSDYMN MTPRRPGPTR KHYQPYAPPR |
| | DFAAYRSKKV AKKPTNKAPH PKQEPQEINF PDDLPGSNTA APVQETLHGC |
| | QPVTQEDGKE SRISVQERQ |
| SEQ ID | |
| NO: 154 | |
| designation | CD40 |
| Sequence | KKVAKKPTNK APHPKQEPQE INFPDDLPGS NTAAPVQETL HGCQPVTQED |
| | GKESRISVQE RQ |
| SEQ ID | |
| NO: 155 | |
| designation | CD40_tandem |
| Sequence | KKVAKKPTNK APHPKQEPQE INFPDDLPGS NTAAPVQETL HGCQPVTQED |
| | GKESRISVQE RQKKVAKKPT NKAPHPKQEP QEINFPDDLP GSNTAAPVQE |
| | TLHGCQPVTQ EDGKESRISV QERQ |
| SEQ ID | |
| NO: 156 | |
| designation | CD40_P227A |
| Sequence | KKVAKKPTNK AAHPKQEPQE INFPDDLPGS NTAAPVQETL HGCQPVTQED |
| | GKESRISVQE RQ |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

Coculture assay set up. T cells from 2 healthy donors were either modified to express the constructs tested or left non-transduced (NTD) at MOI 10. One day prior to coculture set up, effector T cells were thawed and resuspended at 1×10⁶ cells/mL in T cell media (TCM) without IL2 and incubated overnight at 37° C. with 5% CO2. On the day of coculture, T cells (effectors) and Ba/F3-OKT3 targets were collected and counted using a ViCELL BLU as per manufacturer's instructions. T cells were then cocultured with Ba/F3 OKT3 targets at the 10:1, 1:1 and 1:10 E:T (effector: target) ratios overnight. For the inducible costimulatory protein constructs tested (i.e., pIB1097 to pIB1102), an additional set of wells were setup to which 10 μg/mL pembrolizumab was added in addition to Ba/F3 OKT3 targets. Each condition was performed in duplicates. Unstimulated T cells served as negative controls. Two sets of each E:T ratio as well as the T cell only control plates were set up. Brefeldin A was added at 1:1000 dilution to one set of plates to assess cytokine production by intracellular cytokine staining (ICS) using a flow cytometer after overnight co-culture. The second set was incubated for 5 days following which T cell counts and activation marker expression (i.e., 41BB and CD69) was assessed by flow cytometry.

The invention includes modifying components of the TCR complex and associated signaling adaptors (such as, for example, in a TCR incorporated antigen agnostic receptor “TIAAR”), identifying transmembrane domains (TMDs) and modifications that enable constitutive activation of receptors (“constitutive”) and utilizing antibodies to induce activation of the receptor (“inducible”).

scFV targeting co-stimulatory or inhibitor receptors and ligands. The scFV are derived from antibodies targeting co-stimulatory or inhibitory molecules expressed on immune cells.

| Signal peptide | Tag | ECD_TMD | ICD | Costim | GOI description |
|---|---|---|---|---|---|
| CD8a | Myc | hZ270_HL | NA | CD28-CD40 | anti-NKG2A blocking |
| CD8a | Myc | hZ270_LH | NA | CD28-CD40 | anti-NKG2A blocking |
| CD8a | Myc | Varlilumab_HL | NA | CD28-CD40 | anti-CD27 agonist |
| CD8a | Myc | Varlilumab_LH | NA | CD28-CD40 | anti-CD27 agonist |
| CD8a | Myc | Urelumab_HL | NA | CD28-CD40 | anti-CD137 agonist |
| CD8a | Myc | Urelumab_LH | NA | CD28-CD40 | anti-CD137 agonist |
| CD8a | Myc | TRX518_HL | NA | CD28-CD40 | anti-GITR agonist |
| CD8a | Myc | TRX518_LH | NA | CD28-CD40 | anti-GITR agonist |
| CD8a | Myc | Pembroluzimab_HL | NA | CD28-CD40 | anti-PD1 blocking |
| CD8a | Myc | Pembroluzimab_LH | NA | CD28-CD40 | anti-PD1 blocking |
| CD8a | Myc | Atezolizumab_HL | NA | CD28-CD40 | anti-PDL1 blocking |
| CD8a | Myc | Atezolizumab_LH | NA | CD28-CD40 | anti-PDL1 blocking |
| CD8a | Myc | RONK203_HL | NA | CD28-CD40 | anti-FasL blocking |
| CD8a | Myc | RONK203_LH | NA | CD28-CD40 | anti-FasL blocking |
| CD8a | Myc | Tavolimab_HL | NA | CD28-CD40 | anti-OX40 agonist |
| CD8a | Myc | Tavolimab_LH | NA | CD28-CD40 | anti-OX40 agonist |
| CD8a | Myc | Ipilimumab_HL | NA | CD28-CD40 | anti-CTLA4 blocking |
| CD8a | Myc | Ipilimumab_LH | NA | CD28-CD40 | anti-CTLA4 blocking |
| CD8a | Myc | KY1044_HL | NA | CD28-CD40 | anti-ICOS agonist |
| CD8a | Myc | KY1044_LH | NA | CD28-CD40 | anti-ICOS agonist |
| CD8a | Myc | APX005_HL | NA | CD28-CD40 | anti-CD40 agonist |
| CD8a | Myc | APX005_LH | NA | CD28-CD40 | anti-CD40 agonist |
| CD8a | Myc | Selicrelumab_HL | NA | CD28-CD40 | anti-CD40 agonist |
| CD8a | Myc | Selicrelumab_LH | NA | CD28-CD40 | anti-CD40 agonist |

TIAAR (TCR Incorporated) List of Constructs

| | | Signal | | ECD_ | | | |
|---|---|---|---|---|---|---|---|
| Code | Concept | peptide | Tag | TMD | ICD | Costim | GOI description |
| pIB1026 | TIAAR | CD3D | Myc | CD3D | N/A | CD28- | CD3D_CD3D_CD28CD |
| | | | | | | CD40 | 40 |
| pIB1027 | TIAAR | CD3E | FLAG | CD3E | N/A | CD28- | CD3E_CD3E_CD28CD40 |
| | | | | | | CD40 | |
| pIB1028 | TIAAR | CD3G | Myc | CD3G | N/A | CD28- | CD3G_CD3G_CD28CD |
| | | | | | | CD40 | 40 |
| pIB1029 | TIAAR | CD3Z | Myc IC | CD3Z | N/A | CD28- | CD3Z_CD3Z_CD28CD |
| | | | | | | CD40 | 40_Myc |
| pIB1030 | TIAAR | CD8A | Myc | hTRDC | N/A | CD28- | CD8A_hTRDC_CD28C |
| | | | | | | CD40 | D40 |
| pIB1031 | TIAAR | CD8A | FLAG | hTRGC1 | N/A | CD28- | CD8A_hTRGC1_CD28C |
| | | | | | | CD40 | D40 |
| pIB1032 | TIAAR | CD8A | Myc | mTRAC | N/A | CD28- | CD8A_mTRAC_CD28C |
| | | | | | | CD40 | D40 |
| pIB1033 | TIAAR | CD8A | FLAG | mTRBC1 | N/A | CD28- | CD8A_mTRBC1_CD28 |
| | | | | | | CD40 | CD40 |
| pIB1046 | TIAAR | CD8A × 2 | Myc | hTRDC_ | N/A | CD28- | CD8A_hTRDC_CD28C |
| | | | and | hTRGC1 | | CD40 | D40-T2A- |
| | | | FLAG | | | | CD8a_hTRGC1_CD28C |
| | | | | | | | D40 |
| pIB1047 | TIAAR | CD8A × 2 | Myc | mTRAC_ | N/A | CD28- | CD8A_mTRAC_CD28C |
| | | | and | mTRBC1 | | CD40 | D40-T2A- |
| | | | FLAG | | | | CD8A_mTRBC1_CD28 |
| | | | | | | | CD40 |
| pIB1048 | TIAAR | CD3D and | Myc | CD3D_ | N/A | CD28- | CD3D_CD3D_CD28CD |
| | | CD3E | and | CD3E | | CD40 | 40-T2A- |
| | | | FLAG | | | | CD3E_CD3E_CD28CD40 |
| pIB1049 | TIAAR | CD3G and | Myc | CD3G_ | N/A | CD28- | CD3G_CD3G_CD28CD |
| | | CD3E | and | CD3E | | CD40 | 40-T2A- |
| | | | FLAG | | | | CD3E_CD3E_CD28CD40 |
| pIB1050 | TIAAR | CD3D and | Myc | CD3D_CD3E | CD3D_CD3E | CD28- | CD3D_CD3D_CD3D |
| | | CD3E | and | | | CD40 | (ICD)_CD28CD40-T2A- |
| | | | FLAG | | | | CD3E_CD3E_CD3E |
| | | | | | | | (ICD)_CD28CD40 |
| pIB1051 | TIAAR | CD3D and | Myc | CD3D_CD3E | CD3D_CD3E | N/A | CD3D_CD3D_CD3D |
| | | CD3E | and | | | | ICD-T2A- |
| | | | FLAG | | | | CD3E_CD3E_CD3E |
| | | | | | | | ICD |
| pIB1052 | TIAAR | CD3D and | Myc | CD3D_ | N/A | N/A | CD3D_CD3D (control)- |
| | | CD3E | and | CD3E | | | T2A-CD3E_CD3E |
| | | | FLAG | | | | (control) |
| pIB1053 | TIAAR | CD3G and | Myc | CD3G_CD3E | CD3G_CD3E | CD28- | CD3G_CD3G_CD3G |
| | | CD3E | and | | | CD40 | (ICD)_CD28CD40-T2A- |
| | | | FLAG | | | | CD3E_CD3E_CD3E |
| | | | | | | | (ICD)_CD28CD40 |
| pIB1054 | TIAAR | CD3G and | Myc | CD3G_CD3E | CD3G_CD3E | N/A | CD3G_CD3G_CD3G |
| | | CD3E | and | | | | ICD-T2A- |
| | | | FLAG | | | | CD3E_CD3E_CD3E |
| | | | | | | | ICD |
| pIB1055 | TIAAR | CD3G and | Myc | CD3G_C | N/A | N/A | CD3G_CD3G (control)- |
| | | CD3E | and | D3E | | | T2A-CD3E_CD3E |
| | | | FLAG | | | | (control) |
| pIB1056 | TIAAR | CD3Z | Myc IC | CD3Z | CD3Z | CD28- | CD3z_CD3z_CD3z ICD_ |
| | | | | | | CD40 | CD28CD40_Myc |
| pIB1057 | TIAAR | CD3Z | Myc IC | CD3Z | CD3Z | N/A | CD3z_CD3z_CD3z |
| | | | | | | | ICD_Myc |
| pIB1058 | TIAAR | CD3Z | Myc IC | CD3Z | N/A | N/A | CD3z _CD3z (control) |
| pIB1059 | TIAAR | CD3Z | Myc IC | CD3Z | CD3Z | CD28- | CD3Z_CD3Z_CD3Z |
| | | | | | (×2) | CD40 | ICD (duplicating CD3z |
| | | | | | | | endodomain-6 ITAMs) |
| | | | | | | | CD28CD40_Myc |
| pIB1060 | TIAAR | CD3Z | Myc IC | CD3Z | CD3Z | N/A | CD3Z_CD3Z_CD3Z |
| | | | | | (×2) | | ICD (duplicating CD3z |
| | | | | | | | endodomain-6 |
| | | | | | | | ITAMs)_Myc |
| pIB1061 | TIAAR | CD3Z | Myc IC | CD3Z | CD3Z | CD28- | CD3Z_CD3Z_CD28CD40_ |
| | | | | | (×2) | CD40 | CD3Z (6 ITAMs)_Myc |
| | | | | | | (swaped) | |
| pIB1062 | TIAAR | CD3Z | Myc IC | CD3Z | CD3Z | CD28- | CD3Z_CD3Z_CD28CD40_ |
| | | | | | | CD40 | CD3Z ICD_Myc |
| | | | | | | (swaped) | |
| pIB1063 | TIAAR | CD80 | Myc | CD80 | CD80 | N/A | CD80 (control) |
| pIB1064 | TIAAR | no signal | Myc IC | N/A | Lck | N/A | Lck (control) |
| | | peptide | | | | | |
| pIB1065 | TIAAR | no signal | Myc IC | N/A | Lck | N/A | Lck |
| | | peptide | | | (Y505F) | | (Y505F) (control) |
| pIB1066 | TIAAR | CD80 | Myc | CD80 | Lck | N/A | CD80_Lck |
| pIB1067 | TIAAR | CD80 | Myc | CD80 | Lck | CD28- | CD80_Lck_CD28CD40 |
| | | | | | | CD40 | |
| pIB1068 | TIAAR | CD80 | Myc | CD80 | CD80_ | N/A | CD80_Lck (Y505F) |
| | | | | | Lck | | |
| | | | | | (Y505F) | | |
| pIB1069 | TIAAR | CD80 | Myc | CD80 | CD80_ | CD28- | CD80_Lck |
| | | | | | Lck | CD40 | (Y505F)_CD28CD40 |
| | | | | | (Y505F) | | |
| pIB1070 | TIAAR | CD8A | Myc | LAT | LAT | N/A | LAT (control) |
| pIB1071 | TIAAR | CD8A | Myc | LAT | LAT | CD28- | LAT_C28CD40 |
| | | | | | | CD40 | |
| pIB1072 | TIAAR | CD4 | Myc | CD4 | CD4 | CD28- | CD4 control |
| | | | | | | CD40 | |
| pIB1073 | TIAAR | CD4 | Myc | CD4 | CD4 | CD28- | CD4_CD28_CD40 |
| | | | | | | CD40 | |
| pIB1074 | TIAAR | CD8A and | Myc | CD8A | CD8A | N/A | CD8 control |
| | | CD8B | and | and | and | | |
| | | | FLAG | CD8B | CD8B | | |
| pIB1075 | TIAAR | CD8A and | Myc | CD8A | CD8A | CD28- | CD8_CD28_CD40 |
| | | CD8B | and | and | and | CD40 | |
| | | | FLAG | CD8B | CD8B | | |

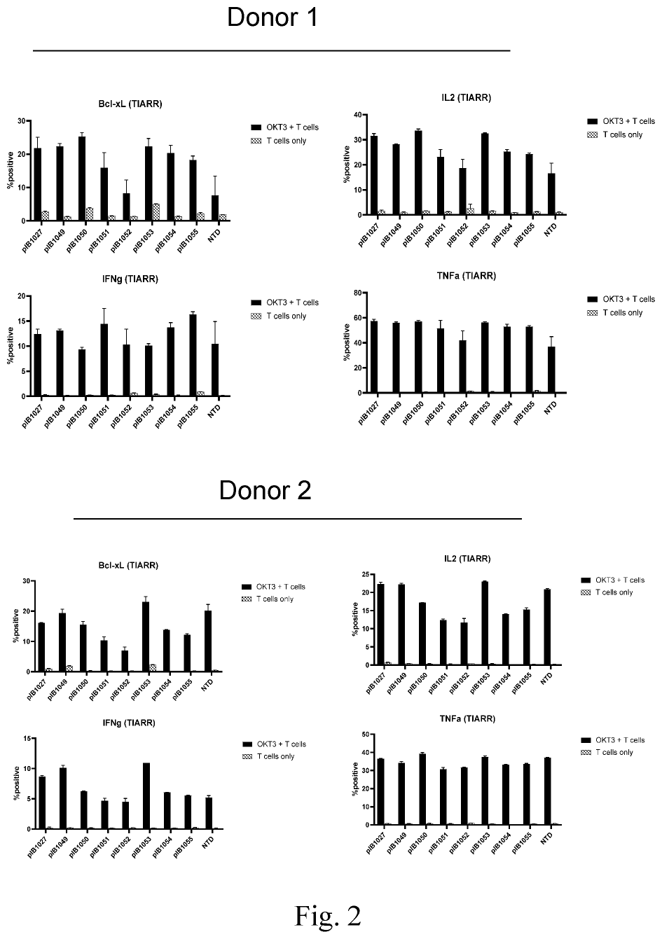

Cytokine production (Bcl-xL, IL2, IFNgamma and TNFalpha) from genetically modified and non-transduced T cells (NTD) after overnight stimulation with either Ba/F3 OKT3 targets or left unstimulated (i.e., T cells only) (FIG. 2). There was increased Bcl-xL, IL2, IFNgamma and TNFalpha production from genetically modified T cells as compared to NTD cells in Donor 1. There was an increase in IFNγ production and comparable or lower levels of Bcl-xL, IL2, IFNgamma and TNFalpha production in genetically modified as compared to NTD cells in Donor 2.

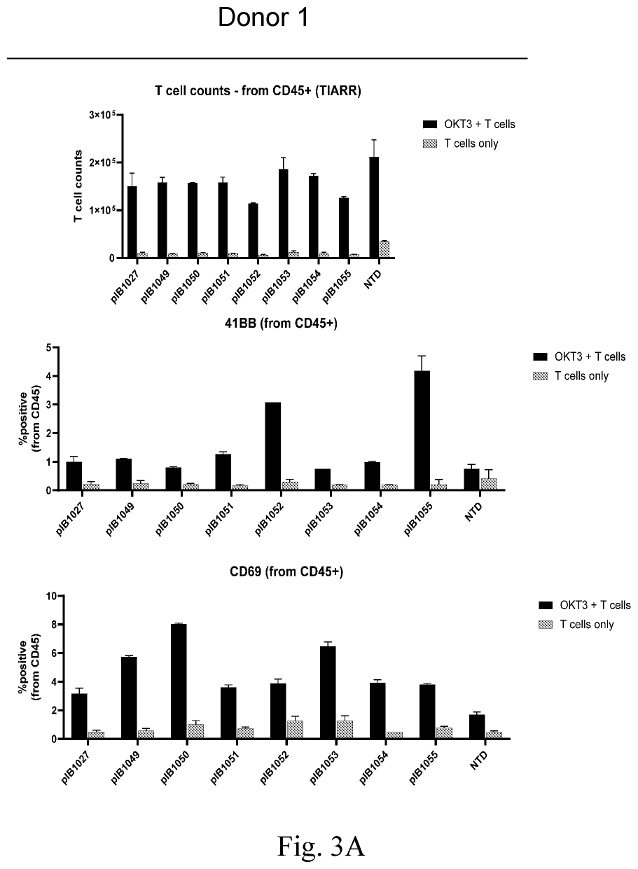

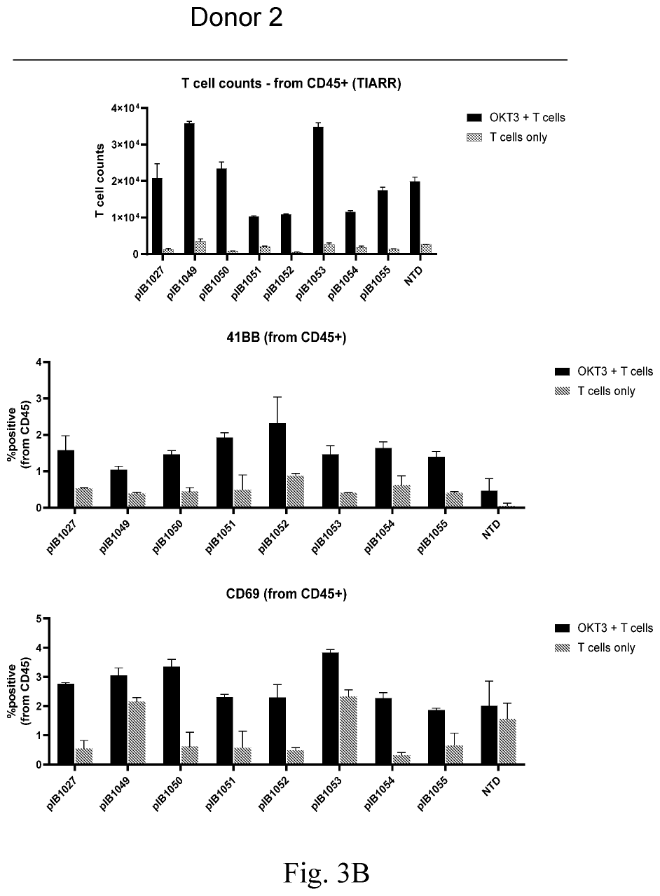

Proliferation (T cell counts from CD45+ (TIARR)) and activation marker expression (41BB from CD45+ and CD69 from CD45+) from genetically modified and non-transduced T cells (NTD) after 5-day co-culture with either Ba/F3 OKT3 targets or left unstimulated (i.e., T cells only) (FIG. 3). There was decreased T cell counts in genetically modified as compared to NTD cells in Donor 1. There was increased or similar 41BB and CD69 expression in genetically modified as compared to NTD cells in Donor 1. There was increased or similar 41BB and CD69 expression in genetically modified T cells as compared to NTD cells in Donor 1. There was increased T cell counts for 2 modifications tested, and a similar or decreased T cell counts for the remaining genetically modified T cells as compared to NTD cells in Donor 2. There was increased or comparable 41BB and CD169 expression in genetically modified as compared to NTD cells in Donor 2.

List of Constitutive Constructs

| | | Signal | | | | | |
|---|---|---|---|---|---|---|---|
| Code | Concept | peptide | Tag | ECD_TMD | ICD | Costim | GOI description |
| pIB1076 | C-SAAR | CD8A | Myc | LZ (cFos)_EGFR | N/A | CD28- | LZ (cFos)-EGFRTM/JMD- |
| | | | | | | CD40 | CD28-CD40 |
| pIB1077 | C-SAAR | CD8A | Myc | LZ (cFos)_CD28 | N/A | CD28- | LZ (cFos)-CD28TM- |
| | | | | | | CD40 | CD28-CD40 |
| pIB1078 | C-SAAR | CD8A | Myc | LZ (cJun)_EGFR | N/A | CD28- | LZ (cJun)-EGFRTM/JMD- |
| | | | | | | CD40 | CD28-CD40 |
| pIB1079 | C-SAAR | CD8A | Myc | LZ (cJun)_CD28 | N/A | CD28- | LZ (cJun)-CD28TM- |
| | | | | | | CD40 | CD28-CD40 |
| pIB1080 | C-SAAR | CD8A | Myc | LZ (c/EBP)_EGFR | N/A | CD28- | LZ (c/EBP)- |
| | | | | | | CD40 | EGFRTM/JMD-CD28- |
| | | | | | | | CD40 |
| pIB1081 | C-SAAR | CD8A | Myc | LZ (c/EBP)_CD28 | N/A | CD28- | LZ (c/EBP)-CD28TM- |
| | | | | | | CD40 | CD28-CD40 |
| pIB1103 | C-SAAR | GpA | Myc | GpA ECD_TMD | N/A | CD28- | GpA ECD-TMD-CD28- |
| | | | | | | CD40 | CD40 |
| pIB1104 | C-SAAR | GpA | Myc | GpA TMD | N/A | CD28- | GpA TMD-CD28-CD40 |
| | | | | | | CD40 | |
| pIB1105 | C-SAAR | EPOR | Myc | EPOR ECD_TMD | N/A | CD28- | EpoR ECD-TMD-CD28- |
| | | | | | | CD40 | CD40 |
| pIB1106 | C-SAAR | EPOR | Myc | EPOR TMD | N/A | CD28- | EpoR TMD-CD28-CD40 |
| | | | | | | CD40 | |
| pIB1107 | C-SAAR | TPOR | Myc | TPOR ECD_TMD | N/A | CD28- | TPO ECD-TPO (WT) TMD- |
| | | | | | | CD40 | CD28-CD40 |
| pIB1108 | C-SAAR | TPOR | Myc | TPOR TMD | N/A | CD28- | TPO (WT) TMD-CD28- |
| | | | | | | CD40 | CD40 |
| pIB1109 | C-SAAR | TPOR | Myc | TPOR ECD_TMD | N/A | CD28- | TPO ECD-TPO (S505N) |
| | | | | (S505N) | | CD40 | TMD-CD28-CD40 |
| pIB1110 | C-SAAR | TPOR | Myc | TPOR TMD | N/A | CD28-CD40 | TPO (S505N) TMD-CD28-CD40 |
| | | | | (S505N) | | | |
| pIB1111 | C-SAAR | TPOR | Myc | TPOR ECD_TMD | N/A | CD28- | TPO ECD-TPO (W515K) |
| | | | | (W515K) | | CD40 | TMD-CD28-CD40 |
| pIB1112 | C-SAAR | TPOR | Myc | TPOR TMD | N/A | CD28- | TPO (W515K) TMD- |
| | | | | (W515K) | | CD40 | CD28-CD40 |
| pIB1113 | C-SAAR | TPOR | Myc | TPOR ECD_TMD | N/A | CD28- | TPO ECD-TPO (H499L) |
| | | | | (H499L) | | CD40 | TMD-CD28-CD40 |
| pIB1114 | C-SAAR | TPOR | Myc | TPOR TMD | N/A | CD28-CD40 | TPO (H499L) TMD-CD28-CD40 |
| | | | | (H499L) | | | |
| pIB1115 | C-SAAR | TPOR | Myc | TPOR ECD_TMD | N/A | CD28- | TPO ECD-TPO (S505N- |
| | | | | (S505N-W515K) | | CD40 | W515K) TMD-CD28-CD40 |
| pIB1116 | C-SAAR | TPOR | Myc | TPOR TMD | N/A | CD28- | TPO (S505N-W515K) TMD- |
| | | | | (S505N-W515K) | | CD40 | CD28-CD40 |
| pIB1117 | C-SAAR | TPOR | Myc | TPOR ECD_TMD | N/A | CD28- | TPO ECD-TPO (H499Y- |
| | | | | (H499Y-S505N) | | CD40 | S505N) TMD-CD28-CD40 |
| pIB1118 | C-SAAR | TPOR | Myc | TPOR TMD | N/A | CD28- | TPO (H499Y-S505N) TMD- |
| | | | | (H499Y-S505N) | | CD40 | CD28-CD40 |
| pIB1119 | C-SAAR | TPOR | Myc | TPOR ECD_TMD | N/A | CD28- | TPO ECD-TPO (L498W- |
| | | | | (L498W-H499C) | | CD40 | H499C) TMD-CD28-CD40 |
| pIB1120 | C-SAAR | TPOR | Myc | TPOR TMD | N/A | CD28-CD40 | TPO (L498W-H499C) TMD- |
| | | | | (L498W-H499C) | | | CD28-CD40 |
| pIB1025 | C-SAAR | CD8a | Myc | CD28 | N/A | CD28- | CD28 TM_CD28_CD40 |
| | | | | | | CD40 | |
| pIB1179 | C-SAAR | CD8a | N/A | IgG1 + CD28TM | N/A | CD28- | IgG1(CH2CH3)- |
| | | | | | | CD40 | CD28(TM)-CD28(CoStim)- |
| | | | | | | | CD40(CoStim) |
| pIB1180 | C-SAAR | CD8a | N/A | IgG1mut + CD28TM | N/A | CD28- | IgG1(CH2CH3,mutant)- |
| | | | | | | CD40 | CD28(TM)-CD28(CoStim)- |
| | | | | | | | CD40(CoStim) |
| pIB1181 | C-SAAR | CD8a | N/A | IgG2 + CD28TM | N/A | CD28- | IgG2(CH2CH3)- |
| | | | | | | CD40 | CD28(TM)-CD28(CoStim)- |
| | | | | | | | CD40(CoStim) |
| pIB1182 | C-SAAR | CD8a | N/A | IgG3 + CD28TM | N/A | CD28- | IgG3(CH2CH3)- |
| | | | | | | CD40 | CD28(TM)-CD28(CoStim)- |
| | | | | | | | CD40(CoStim) |
| pIB1183 | C-SAAR | CD8a | N/A | IgG4 + CD28TM | N/A | CD28- | IgG4(CH2CH3)- |
| | | | | | | CD40 | CD28(TM)-CD28(CoStim)- |
| | | | | | | | CD40(CoStim) |
| pIB1184 | C-SAAR | CD8a | N/A | IgG4mut + CD28TM | N/A | CD28- | IgG4(CH2CH3,mutant)- |
| | | | | | | CD40 | CD28(TM)-CD28(CoStim)- |
| | | | | | | | CD40(CoStim) |
| pIB1185 | C-SAAR | CD8a | N/A | IgG1 + CD28 | N/A | CD28- | IgG1(CH2CH3)- |
| | | | | stalk/TM | | CD40 | CD28(Stalk + TM)- |
| | | | | | | | CD28(CoStim)- |
| | | | | | | | CD40(CoStim) |
| pIB1186 | C-SAAR | CD8a | N/A | IgG1mut + CD28 | N/A | CD28- | IgG1(CH2CH3,mutant)- |
| | | | | stalk/TM | | CD40 | CD28(Stalk + TM)- |
| | | | | | | | CD28(CoStim)- |
| | | | | | | | CD40(CoStim) |
| pIB1187 | C-SAAR | CD8a | N/A | IgG2 + CD28 | N/A | CD28- | IgG2(CH2CH3)- |
| | | | | stalk/TM | | CD40 | CD28(Stalk + TM)- |
| | | | | | | | CD28(CoStim)- |
| | | | | | | | CD40(CoStim) |
| pIB1188 | C-SAAR | CD8a | N/A | IgG3 + CD28 | N/A | CD28- | IgG3(CH2CH3)- |
| | | | | stalk/TM | | CD40 | CD28(Stalk + TM)- |
| | | | | | | | CD28(CoStim)- |
| | | | | | | | CD40(CoStim) |
| pIB1189 | C-SAAR | CD8a | N/A | IgG4 + CD28 | N/A | CD28- | IgG4(CH2CH3)- |
| | | | | stalk/TM | | CD40 | CD28(Stalk + TM)- |
| | | | | | | | CD28(CoStim)- |
| | | | | | | | CD40(CoStim) |
| pIB1190 | C-SAAR | CD8a | N/A | IgG4mut + CD28 | N/A | CD28- | IgG4(CH2CH3,mutant)- |
| | | | | stalk/TM | | CD40 | CD28(Stalk + TM)- |
| | | | | | | | CD28(CoStim)- |
| | | | | | | | CD40(CoStim) |

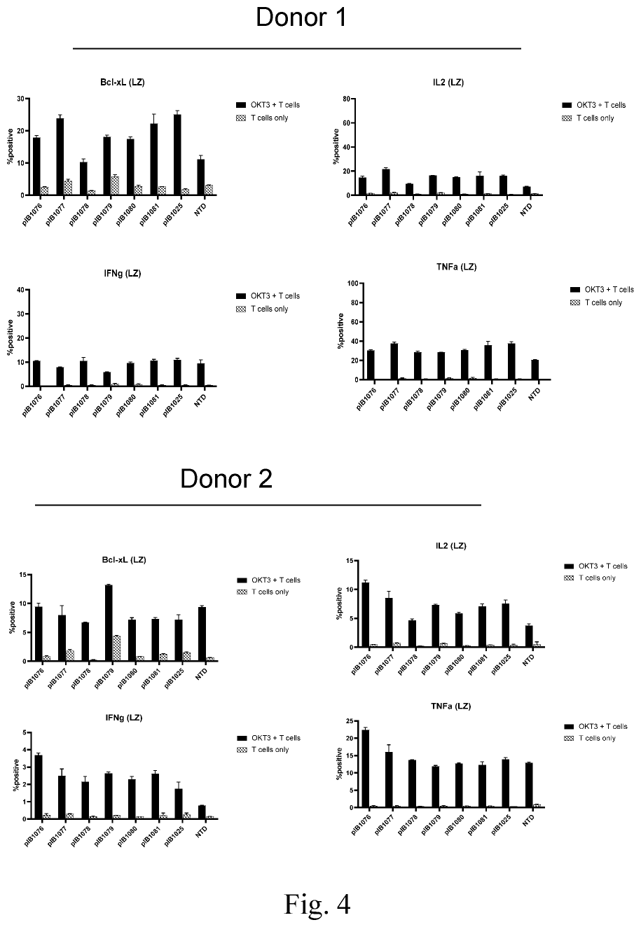

Cytokine production (Bcl-xL, IL2, IFNgamma and TNFalpha) from genetically modified and non-transduced T cells (NTD) after overnight stimulation with either Ba/F3 OKT3 targets or left unstimulated (i.e., T cells only) (FIG. 4). There was increased or comparable Bcl-xL, IL2, and TNFalpha production and comparable or lower levels of IFNgamma from genetically modified T cells as compared to NTD cells in Donor 1. There was an increase in IFNgamma and IL2 production and comparable or lower levels of Bcl-xL and TNFalpha production in genetically modified as compared to NTD cells in Donor 2.

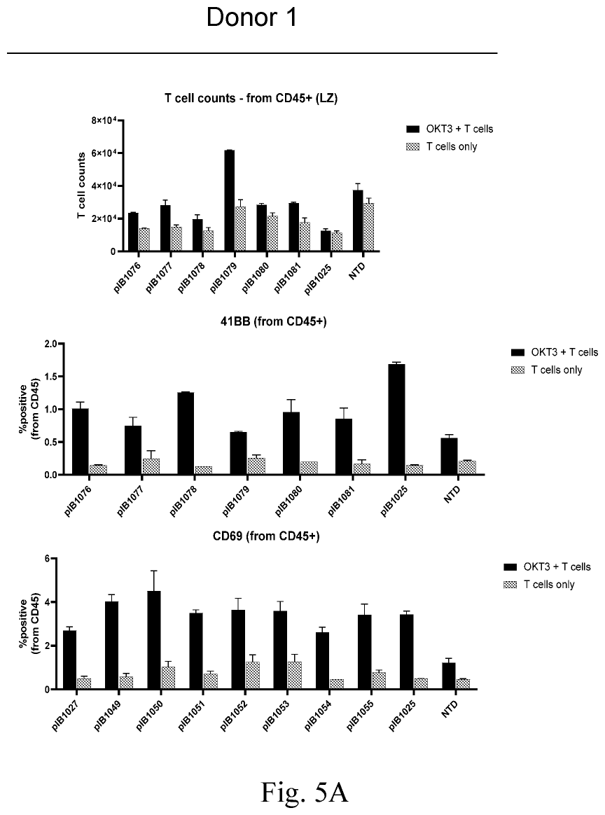

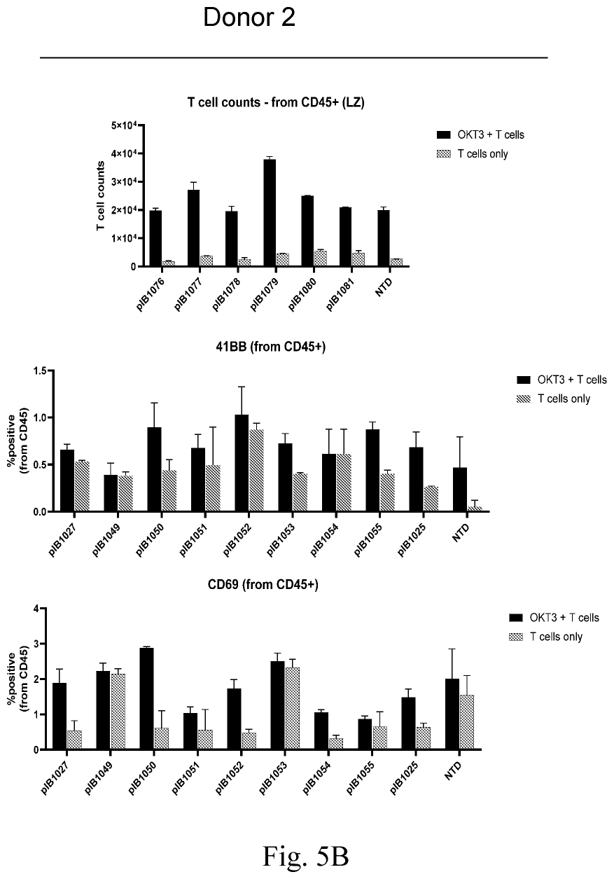

Proliferation (T cell counts from CD45+ (LZ)) and activation marker expression (41BB from CD45+ and CD69 from CD45+) from genetically modified and non-transduced T cells (NTD) after 5-day co-culture with either Ba/F3 OKT3 targets or left unstimulated (i.e., T cells only) (FIG. 5). There was decreased T cell counts in genetically modified as compared to NTD cells in Donor 1. There was increased 41BB and CD69 expression in genetically modified T cells as compared to NTD cells in Donor 1. There was increased T cell counts as compared to NTD cells in Donor 2, an increase in 41BB expression in genetically modified T cells as compared to NTD cells, and similar or decreased expression of CD169 in genetically modified T cells as compared to NTD cells in Donor 2.

List of Inducible Constructs

| | | Signal | | | | | |
|---|---|---|---|---|---|---|---|
| Code | Concept | peptide | Tag | ECD_TMD | ICD | Costim | GOI description |
| pIB1082 | Inducible | EGFR | Myc | EGFR | N/A | CD28- | WT EGFR ECD- |
| | | | | | | CD40 | EGFRTM/JMD-CD28-CD40 |
| pIB1083 | Inducible | EGFR | Myc | EGFR | N/A | CD28- | domain IV-EGFRTM/JMD- |
| | | | | (domain | | CD40 | CD28-CD40 |
| | | | | IV) | | | |
| pIB1084 | Inducible | EGFR | Myc | EGFR | N/A | CD28- | EGFRTM/JMD-CD28-CD40 |
| | | | | (623-668) | | CD40 | (control) |
| pIB1085 | Inducible | Her2 | Myc | Her2 | N/A | CD28- | Her 2 (Domain 1 to IV)- |
| | | | | | | CD40 | TMD/JMD-CD28-CD40 |
| pIB1086 | Inducible | Her2 | Myc | Her2 | N/A | CD28- | Her 2 (Domain 1 to IV)-TMD |
| | | | | (V659E) | | CD40 | (V659E)/JMD-CD28-CD40 |
| pIB1087 | Inducible | Her2 | Myc | Her2 | N/A | CD28- | Her 2 (Domain 1 to IV)-TMD |
| | | | | (V660D) | | CD40 | (G660D)/JMD-CD28-CD40 |
| pIB1088 | Inducible | Her2 | Myc | Her2 | N/A | CD28- | Her 2 (Domain 1 to IV)-TMD |
| | | | | (V660R) | | CD40 | (G660R)/JMD-CD28-CD40 |
| pIB1089 | Inducible | Her2 | Myc | Her2 | N/A | CD28- | Her 2 (Domain IV)- |
| | | | | domain | | CD40 | TMD/JMD-CD28-CD40 |
| | | | | IV TMD | | | |
| pIB1090 | Inducible | Her2 | Myc | Her2 | N/A | CD28- | Her 2 (Domain IV)-TMD |
| | | | | domain | | CD40 | (V659E)/JMD-CD28-CD40 |
| | | | | IV_TMD | | | |
| | | | | (V659E) | | | |
| pIB1091 | Inducible | Her2 | Myc | Her2 | N/A | CD28- | Her 2 (Domain IV)-TMD |
| | | | | domain | | CD40 | (G660D)/JMD-CD28-CD40 |
| | | | | IV_TMD | | | |
| | | | | (G660D) | | | |
| pIB1092 | Inducible | Her2 | Myc | Her2 | N/A | CD28- | Her 2 (Domain IV)-TMD |
| | | | | domain | | CD40 | (G660R)/JMD-CD28-CD40 |
| | | | | IV_TMD | | | |
| | | | | (G660R) | | | |
| pIB1093 | Inducible | Her2 | Myc | Her2 TMD | N/A | CD28- | Her 2 TMD/JMD-CD28- |
| | | | | | | CD40 | CD40 |
| pIB1094 | Inducible | Her2 | Myc | Her2 TMD | N/A | CD28- | Her 2 TMD (V659E)/JMD- |
| | | | | (V659E) | | CD40 | CD28-CD40 |
| pIB1095 | Inducible | Her2 | Myc | Her2 TMD | N/A | CD28- | Her 2 TMD (G660D)/JMD- |
| | | | | (G660D) | | CD40 | CD28-CD40 |
| pIB1096 | Inducible | Her2 | Myc | Her2 TMD | N/A | CD28- | Her 2 TMD (G660R)/JMD- |
| | | | | (G660R) | | CD40 | CD28-CD40 |
| pIB1097 | Inducible | CD8A | Myc | A30514 | N/A | CD28- | Anti-ID1 VH-VL (A30514- |
| | | | | VH_VL | | CD40 | pembro)-CD28TMD CD28- |
| | | | | | | | CD40 |
| pIB1098 | Inducible | CD8A | Myc | A30514 | N/A | CD28- | Anti-ID1 VL-VH (A30514- |
| | | | | VL_VH | | CD40 | pembro)-CD28TMD CD28- |
| | | | | | | | CD40 |
| pIB1099 | Inducible | CD8A | Myc | A30523 | N/A | CD28- | Anti-ID2 Vh-VL (A30523- |
| | | | | VH_VL | | CD40 | pembro)-CD28TMD CD28- |
| | | | | | | | CD40 |
| pIB1100 | Inducible | CD8A | Myc | A30523 | N/A | CD28- | Anti-ID2 VL-Vh (A30523- |
| | | | | VL_VH | | CD40 | pembro)-CD28TMD CD28- |
| | | | | | | | CD40 |
| pIB1101 | Inducible | CD8A | Myc | A30633 | N/A | CD28- | Anti-ID3 Vh-VL (A30633- |
| | | | | VH_VL | | CD40 | pembro)-CD28TMD CD28- |
| | | | | | | | CD40 |
| pIB1102 | Inducible | CD8A | Myc | A30633 | N/A | CD28- | Anti-ID3 VL-VH (A30633- |
| | | | | VL_VH | | CD40 | pembro)-CD28TMD CD28- |
| | | | | | | | CD40 |

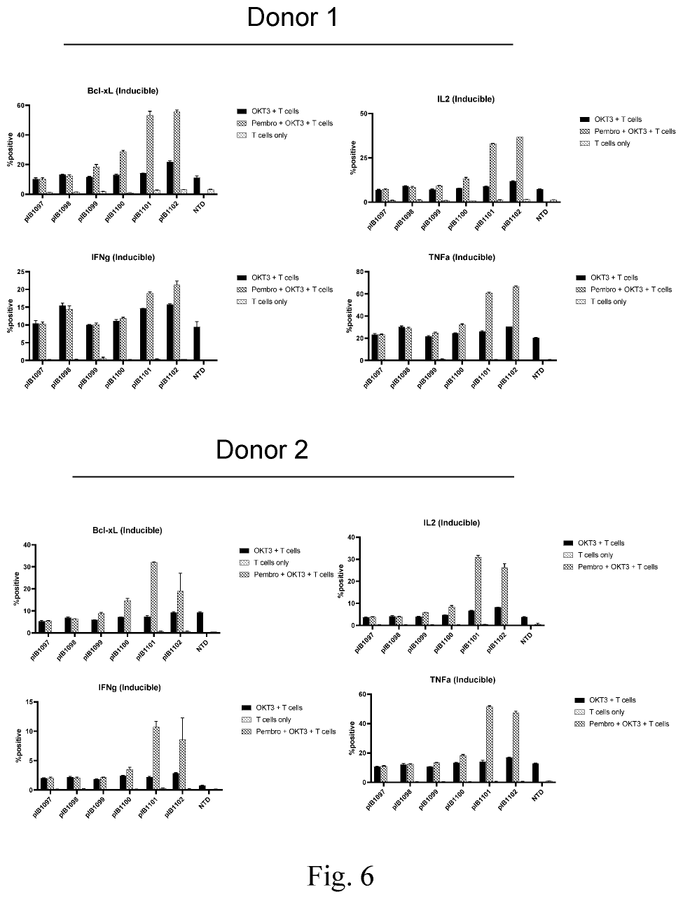

Cytokine production (Bcl-xL, IL2, IFNg and TNFa) from genetically modified and non-transduced T cells (NTD) after overnight stimulation with either Ba/F3 OKT3 targets or Ba/F3 OKT3 targets with 10 ug/mL pembrolizumab or left unstimulated (i.e., T cells only) (FIG. 6). There was increased or comparable Bcl-xL, IL2, IFNgamma and TNFalpha production from genetically modified T cells in the presence of Ba/F3 OKT3 and pembrolizumab as compared to conditions with Ba/F3 OKT3 stimulation alone and NTD cells in both Donor 1 and Donor 2.

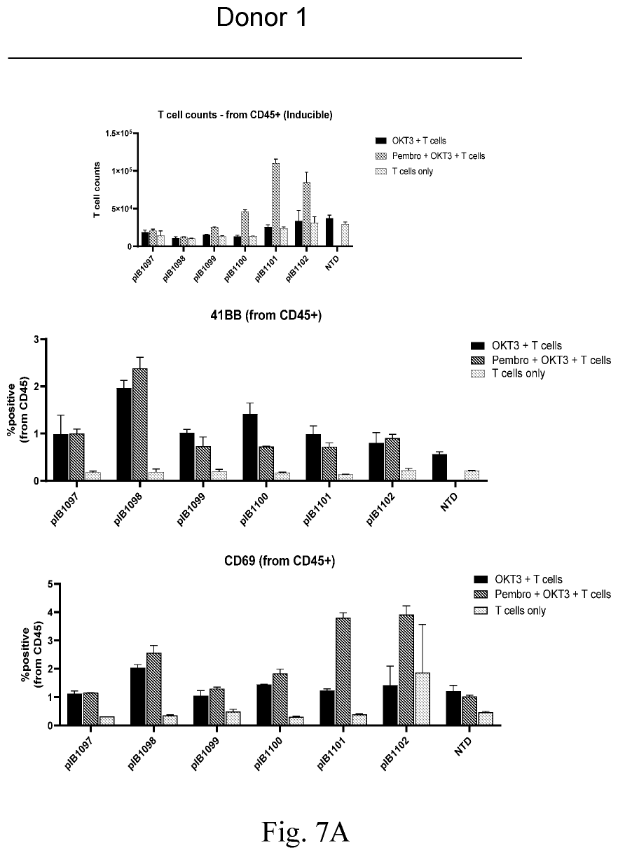

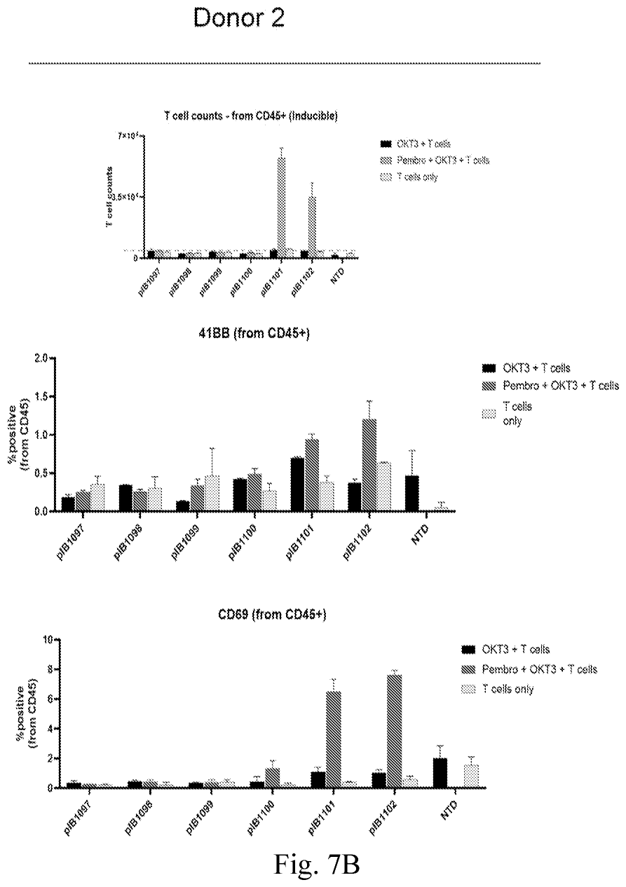

Proliferation (T cell counts from CD45+ (Inducible)) and activation marker expression (41BB from CD45+ and CD69 from CD45+) from genetically modified and non-transduced T cells (NTD) after 5-day co-culture with either Ba/F3 OKT3 targets or Ba/F3 OKT3 targets with 10 ug/mL pembrolizumab or left unstimulated (i.e., T cells only) (FIG. 7). There was increased, comparable and decreased T cell counts in genetically modified T cells as compared to NTD cells in Donor 1. There was increased or similar 41BB and CD169 expression in genetically modified T cells as compared to NTD cells in Donor 1. There was increased or comparable T cell counts in genetically modified T cells as compared to NTD cells in Donor 2. There was increased, similar and decreased 41BB and CD169 expression in genetically modified cells as compared to NTD cells in Donor 2. Increased T cell counts and activation marker expression in both donors were observed when the genetically modified T cells were stimulated with Ba/F3 OKT3 in the presence of pembrolizumab compared to conditions with Ba/F3 OKT3 stimulation alone as well as NTD cells.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An engineered protein comprising a clustering domain and a signaling domain comprising a CD40 signaling domain or signaling fragment thereof, wherein the clustering domain is capable of oligomerization whereby the signaling domain is activated.

2. The engineered protein of claim 1, wherein the clustering domain is capable of oligomerization by self-assembly.

3. The engineered protein of claim 2, where self-assembly comprises formation of a homodimer.

4. The engineered protein of claim 2, wherein self-assembly comprises complex formation with a receptor.

5. The engineered protein of claim 2, wherein oligomerization is mediated by ligand binding.

6. The engineered protein of claims 1 to 4, which comprises a constitutively stimulating antigen agnostic receptor (C-SAAR)

7. The engineered protein of claims 1 to 4, which comprises a C-SAAR of Table 3 or 4.

8. The engineered protein of claims 1 to 4, which comprises LZ(cFos)-EGFRTM/JMD-CD28-CD40, LZ(cFos)-CD28TM-CD28-CD40, LZ(cJun)-EGFRTM/JMD-CD28-CD40, LZ(cJun)-CD28TM-CD28-CD40, LZ(c/EBP)-EGFRTM/JMD-CD28-CD40, or LZ(c/EBP)-CD28TM-CD28-CD40.

9. The engineered protein of claims 1 to 5, which comprises an inducible costimulatory receptor.

10. The engineered protein of claims 1 to 5, which comprises an inducible costimulatory receptor of Table 5 or 6.

11. The engineered protein of claims 1 to 5, which comprises Anti-ID1-VH-VL(A30514-pembro)-CD28TMD-CD28-CD40, or Anti-ID1-VL-VH(A30514-pembro)-CD28TMD-CD28-CD40, or Anti-ID2-VH-VL(A30523-pembro)-CD28TMD-CD28-CD40, or Anti-ID2-VL-Vh(A30523-pembro)-CD28TMD-CD28-CD40, or Anti-ID3-Vh-VL (A30633-pembro)-CD28TMD-CD28-CD40, or Anti-ID3-VL-VH(A30633-pembro)-CD28TMD-CD28-CD40.

12. The engineered protein of claim 1, wherein the engineered protein comprises a transmembrane domain and the clustering domain oligomerizes when the engineered protein is bound by a ligand.

13. The engineered protein of claim 12, wherein the ligand comprises an extracellular ligand.

14. The engineered protein of claim 12, wherein the ligand comprises an intracellular ligand.

15. The engineered protein of claim 1, wherein the engineered protein comprises a transmembrane domain and an extracellular ligand binding domain and the signaling domain is activated when two or more copies of the engineered protein are maintained in proximity to one another by ligand binding.

16. The engineered protein of claim 15, wherein the ligand comprises an antibody.

17. The engineered protein of claim 1-14, wherein the engineered protein comprises a clustering domain and a signaling domain comprising a CD40 intracellular domain, wherein the chimeric protein costimulates a T cell when an endogenous T cell receptor (TCR) engages its cognate antigen.

18. The engineered protein of claim 17, wherein the clustering domain comprises a T-cell receptor (TCR) clustering domain.

19. The engineered protein of claim 17, wherein the clustering domain is activated by binding of an extracellular ligand.

20. The engineered protein of claim 18, wherein the TCR clustering domain comprises a protein component of a TCR complex or a portion thereof.

21. The engineered protein of claim 18, which comprises a T cell receptor integrated antigen agnostic receptor (TIAAR).

22. The engineered protein of claim 18, which comprises a TIAAR from Table 1 or 2.

23. The engineered protein of claim 18, which comprises CD3G_CD3G_CD28CD40 and T2A-CD3E_CD3E_CD28CD40, or CD3D_CD3D_CD3D(ICD)_CD28CD40 and CD3E_CD3E_CD3E(ICD)_CD28CD40, or CD3D_CD3D_CD3D(ICD) and CD3E_CD3E_CD3E(ICD), or CD3G_CD3G_CD3 G(ICD)_CD28CD40 and CD3E_CD3E_CD3E(ICD)_CD28CD40, or CD3G_CD3G_CD3G(ICD) and CD3E_CD3E_CD3E(ICD).

24. The protein of claim 20, wherein the protein of the TCR complex comprises CD3D, CD3E, CD3G, CD3Z, a constant chain of TCR alpha, a constant chain of TCR beta, a constant chain of TCR gamma, or a constant chain of TCR delta.

25. The protein of claim 20 or 24, wherein the portion thereof comprises a transmembrane portion, an extracellular portion, an intracellular portion or a combination thereof.

26. The protein of claim 24 or 25, wherein a constant chain of TCR alpha is co-expressed with the constant chain of TCR beta.

27. The protein of claim 24 or 25, wherein the constant chain of TCR gamma is co-expressed with the constant chain of TCR delta.

28. The protein of any one of claims 1-27, wherein the signaling domain further comprises a costimulatory domain.

29. The protein of claim 28, wherein the costimulatory domain is CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6 or a portion thereof.

30. The protein of claim 29, wherein the costimulatory domain is CD28.

31. The protein of any one of claims 1 to 30, wherein the protein further comprises a signal peptide.

32. A nucleic acid which encodes the protein of any one of claims 1 to 31.

33. A vector which comprises the nucleic acid of claim 32.

34. A cell which expresses the protein of any one of claims 1 to 31.

35. A cell which expresses two or more of the proteins of claims 1 to 31.

36. The cell of claim 34 or 35, wherein the cell is an alpha-beta T cell.

37. The cell of claim 36, wherein the alpha-beta T cell is a tumor infiltrating lymphocyte (TIL).

38. The cell of claim 36 or 37, wherein the TCR clustering domain comprises a constant chain of TCR gamma is co-expressed with a constant chain of TCR delta.

39. The cell of claim 34 or 35, wherein the cell is a gamma-delta T cell.

40. The cell of claim 39, wherein the TCR clustering domain comprises a constant chain of TCR alpha is co-expressed with a constant chain of TCR beta.

41. A method of making the cell of any one of claims 34 to 40, which comprises the step of transducing or transfecting a cell with a vector of claim 33.

42. A method for preparing a population of cells that express a protein of any one of claims 1 to 31 comprising detecting expression of the protein on the surface of cells transfected or transduced with a vector according to claim 33 and selecting cells which are identified as expressing the protein.

43. A cell population produced by the method of claim 42.

44. A cell population which is enriched for cell expression a protein of any one of claims 1 to 31.

45. A method for treating a disease in a subject in need thereof, which comprises the step of administering the cell of any one of claims 34 to 40 or the cell population of claim 43 or 23 to the subject.


SEQUENCE LISTING (158 sequences)

SEQ ID NO: 1 - protein, 21 aa, Artificial Sequence
  synthetic construct
  MISC_FEATURE (1)..(21): signal peptide
MEHSTFLSGLVLATLLSQVSP

SEQ ID NO: 2 - protein, 105 aa, Artificial Sequence
  synthetic construct
  MISC_FEATURE (1)..(105): CD3D ECD-TMD
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFA

SEQ ID NO: 3 - protein, 103 aa, Artificial Sequence
  synthetic construct
  MISC_FEATURE (1)..(103): CD28-CD40 costimulatory domain
RSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 4 - protein, 229 aa, Artificial Sequence
  synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 5 - protein, 22 aa, Artificial Sequence
  synthetic construct
  MISC_FEATURE (1)..(22): signal peptide
MQSGTHWRVLGLCLLSVGVWGQ

SEQ ID NO: 6 - protein, 130 aa, Artificial Sequence
  synthetic construct
  MISC_FEATURE (1)..(130): CD3E ECD-TMD
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWS

SEQ ID NO: 7 - protein, 103 aa, Artificial Sequence; identical to SEQ ID NO: 3
  synthetic construct
  MISC_FEATURE (1)..(103): CD28-CD40 costimulatory domain

SEQ ID NO: 8 - protein, 255 aa, Artificial Sequence
  synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 9 - protein, 22 aa, Artificial Sequence
  synthetic construct
  MISC_FEATURE (1)..(22): signal peptide
MEQGKGLAVLILAIILLQGTLA

SEQ ID NO: 10 - protein, 115 aa, Artificial Sequence
  synthetic construct
  MISC_FEATURE (1)..(115): CD3G ECD-TMD
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIA

SEQ ID NO: 11 - protein, 103 aa, Artificial Sequence; identical to SEQ ID NO: 3
  synthetic construct
  MISC_FEATURE (1)..(103): CD28-CD40 costimulatory domain

SEQ ID NO: 12 - protein, 240 aa, Artificial Sequence
  Synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 13 - protein, 21 aa, Artificial Sequence
  Synthetic construct
  MISC_FEATURE (1)..(21): signal peptide
MKWKALFTAAILQAQLPITEA

SEQ ID NO: 14 - protein, 30 aa, Artificial Sequence
  Synthetic construct
  MISC_FEATURE (1)..(30): CD3Z ECD-TMD
QSFGLLDPKLCYLLDGILFIYGVILTALFL

SEQ ID NO: 15 - protein, 103 aa, Artificial Sequence; identical to SEQ ID NO: 3
  Synthetic construct
  MISC_FEATURE (1)..(103): CD28-CD40 costimulatory domain

SEQ ID NO: 16 - protein, 154 aa, Artificial Sequence
  synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 17 - protein, 21 aa, Artificial Sequence
  synthetic construct
  MISC_FEATURE (1)..(21): signal peptide
MALPVTALLLPLALLLHAARP

SEQ ID NO: 18 - protein, 153 aa, Artificial Sequence
  synthetic construct
  misc (1)..(153): TRDC ECD-TMD
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 19 - protein, 103 aa, Artificial Sequence; identical to SEQ ID NO: 3
  synthetic construct
  MISC_FEATURE (1)..(103): CD28-CD40 costimulatory domain

SEQ ID NO: 20 - protein, 277 aa, Artificial Sequence
  synthetic construct
MALPVTALLLPLALLLHAARPSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 21 - protein, 21 aa, Artificial Sequence
  synthetic construct
  MISC_FEATURE (1)..(21): signal peptide
MALPVTALLLPLALLLHAARP

SEQ ID NO: 22 - protein, 161 aa, Artificial Sequence
  synthetic construct
  MISC_FEATURE (1)..(161): TRGC1 ECD-TMD
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLL

SEQ ID NO: 23 - protein, 103 aa, Artificial Sequence; identical to SEQ ID NO: 3
  synthetic construct
  MISC_FEATURE (1)..(103): CD28-CD40 costimulatory domain

SEQ ID NO: 24 - protein, 285 aa, Artificial Sequence
  synthetic construct
MALPVTALLLPLALLLHAARPDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 25 - protein, 21 aa, Artificial Sequence
  synthetic construct
  MISC_FEATURE (1)..(21): signal peptide
MALPVTALLLPLALLLHAARP

SEQ ID NO: 26 - protein, 136 aa, Artificial Sequence
  synthetic construct
  MISC_FEATURE (1)..(136): TRAC ECD-TMD
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 27 - protein, 103 aa, Artificial Sequence; identical to SEQ ID NO: 3
  synthetic construct
  MISC_FEATURE (1)..(103): CD28-CD40 costimulatory domain

SEQ ID NO: 28 - protein, 260 aa, Artificial Sequence
  synthetic construct
MALPVTALLLPLALLLHAARPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 29 - protein, 21 aa, Artificial Sequence
  synthetic construct
  MISC_FEATURE (1)..(21): signal peptide
MALPVTALLLPLALLLHAARP

SEQ ID NO: 30 - protein, 169 aa, Artificial Sequence
  synthetic construct
  MISC_FEATURE (1)..(169): TRBC1 ECD-TMD
VLTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 103 aa, Artificial Sequence; identical to SEQ ID NO: 3
  synthetic construct
  MISC_FEATURE (1)..(103): CD28-CD40 costimulatory domain

SEQ ID NO: 32 - protein, 293 aa, Artificial Sequence
  synthetic construct
MALPVTALLLPLALLLHAARPVLTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 33 - protein, 549 aa, Artificial Sequence
  synthetic construct
MALPVTALLLPLALLLHAARPEQKLISEEDLQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 34 - protein, 549 aa, Artificial Sequence
  synthetic construct
MALPVTALLLPLALLLHAARPEQKLISEEDLDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 35 - protein, 543 aa, Artificial Sequence
  synthetic construct
MALPVTALLLPLALLLHAARPEQKLISEEDLQVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSGGGGSGGGGSGGGGSDIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 36 - protein, 543 aa, Artificial Sequence
  synthetic construct
MALPVTALLLPLALLLHAARPEQKLISEEDLDIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKGGGGSGGGGSGGGGSQVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 37 - protein, 547 aa, Artificial Sequence
  synthetic construct
MALPVTALLLPLALLLHAARPEQKLISEEDLQVQLQQWGAGLLKPSETLSLTCAVYGGSFSGYYWSWIRQSPEGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 38 - protein, 547 aa, Artificial Sequence
  synthetic construct
MALPVTALLLPLALLLHAARPEQKLISEEDLEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRGGGGSGGGGSGGGGSQVQLQQWGAGLLKPSETLSLTCAVYGGSFSGYYWSWIRQSPEGLEWIGEINHGGYVTYNPSLESRVTISVDTSKNQFSLKLSSVTAADTAVYYCARDYGPGNYDWYFDLWGRGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 39 - protein, 543 aa, Artificial Sequence
  synthetic construct
MALPVTALLLPLALLLHAARPEQKLISEEDLQVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSGGGGSGGGGSGGGGSEIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 40 - protein, 543 aa, Artificial Sequence
  synthetic construct
MALPVTALLLPLALLLHAARPEQKLISEEDLEIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIKGGGGSGGGGSGGGGSQVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 41 - protein, 549 aa, Artificial Sequence
  synthetic construct
MALPVTALLLPLALLLHAARPEQKLISEEDLQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 42 - protein, 549 aa, Artificial Sequence
  synthetic construct
MALPVTALLLPLALLLHAARPEQKLISEEDLEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 43 - protein, 543 aa, Artificial Sequence
  synthetic construct
MALPVTALLLPLALLLHAARPEQKLISEEDLEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 44 - protein, 543 aa, Artificial Sequence
  synthetic construct
MALPVTALLLPLALLLHAARPEQKLISEEDLDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 45 - protein, 549 aa, Artificial Sequence
  synthetic construct
MALPVTALLLPLALLLHAARPEQKLISEEDLQVQLVQSGAEVKKPGASVKVSCKASGYTFTNYWIGWVKQAPGQGLEWIGYLYPGGLYTNYNEKFKGKATMTADTSTNTAYMELSSLRSEDTAVYYCARYRDYDYAMDYWGQGTLVTVSSGGGGSGGGGSGGGGSDVVMTQTPLSLPVTLGQPASISCKSTKSLLNSDGFTYLGWCLQKPGQSPQLLIYLVSNRFSGVPDRFSGSGSGTDFTLKISRVEAEDVGVYYCFQSNYLPLTFGQGTKLEIKRAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 46 - protein, 549 aa, Artificial Sequence
  synthetic construct
MALPVTALLLPLALLLHAARPEQKLISEEDLDVVMTQTPLSLPVTLGQPASISCKSTKSLLNSDGFTYLGWCLQKPGQSPQLLIYLVSNRFSGVPDRFSGSGSGTDFTLKISRVEAEDVGVYYCFQSNYLPLTFGQGTKLEIKRGGGGSGGGGSGGGGSQVQLVQSGAEVKKPGASVKVSCKASGYTFTNYWIGWVKQAPGQGLEWIGYLYPGGLYTNYNEKFKGKATMTADTSTNTAYMELSSLRSEDTAVYYCARYRDYDYAMDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 47 - protein, 545 aa, Artificial Sequence
  synthetic construct
MALPVTALLLPLALLLHAARPEQKLISEEDLQVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSGGGGSGGGGSGGGGSDIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 48 - protein, 545 aa, Artificial Sequence
  synthetic construct
MALPVTALLLPLALLLHAARPEQKLISEEDLDIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKGGGGSGGGGSGGGGSQVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 49 - protein, 543 aa, Artificial Sequence
  synthetic construct
MALPVTALLLPLALLLHAARPEQKLISEEDLQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 50 - protein, 543 aa, Artificial Sequence
  synthetic construct
MALPVTALLLPLALLLHAARPEQKLISEEDLEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 51 - protein, 549 aa, Artificial Sequence
  synthetic construct
MALPVTALLLPLALLLHAARPEQKLISEEDLEVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSGGGGSGGGGSGGGGSEIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 52 - protein, 549 aa, Artificial Sequence
  synthetic construct
MALPVTALLLPLALLLHAARPEQKLISEEDLEIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKGGGGSGGGGSGGGGSEVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 53 - protein, 545 aa, Artificial Sequence
  synthetic construct
MALPVTALLLPLALLLHAARPEQKLISEEDLQVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSGGGGSGGGGSGGGGSDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 54 - protein, 545 aa, Artificial Sequence
  synthetic construct
MALPVTALLLPLALLLHAARPEQKLISEEDLDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKGGGGSGGGGSGGGGSQVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 55 - protein, 550 aa, Artificial Sequence
  synthetic construct
MALPVTALLLPLALLLHAARPEQKLISEEDLQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 56 - protein, 550 aa, Artificial Sequence
  synthetic construct
MALPVTALLLPLALLLHAARPEQKLISEEDLDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 57 - protein, 293 aa, Artificial Sequence
  synthetic construct
MALPVTALLLPLALLLHAARPEQKLISEEDLILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 58 - protein, 239 aa, Artificial Sequence
  synthetic construct
MEHSTFLSGLVLATLLSQVSPEQKLISEEDLFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 59 - protein, 263 aa, Artificial Sequence
  synthetic construct
MQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 60 - protein, 250 aa, Artificial Sequence
  synthetic construct
MEQGKGLAVLILAIILLQGTLAEQKLISEEDLQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 61 - protein, 164 aa, Artificial Sequence
  synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQEQKLISEEDL

SEQ ID NO: 62 - protein, 287 aa, Artificial Sequence
  synthetic construct
MALPVTALLLPLALLLHAARPEQKLISEEDLSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 63 - protein, 293 aa, Artificial Sequence
  synthetic construct
MALPVTALLLPLALLLHAARPDYKDDDDKDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 64 - protein, 270 aa, Artificial Sequence
  synthetic construct
MALPVTALLLPLALLLHAARPEQKLISEEDLIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 65 - protein, 301 aa, Artificial Sequence
  synthetic construct
MALPVTALLLPLALLLHAARPDYKDDDDKVLTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 66 - protein, 605 aa, Artificial Sequence
  synthetic construct
MALPVTALLLPLALLLHAARPEQKLISEEDLSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMALPVTALLLPLALLLHAARPDYKDDDDKDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 67 - protein, 596 aa, Artificial Sequence
  synthetic construct
MALPVTALLLPLALLLHAARPEQKLISEEDLIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMALPVTALLLPLALLLHAARPDYKDDDDKVLTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 68 - protein, 527 aa, Artificial Sequence
  synthetic construct
MEHSTFLSGLVLATLLSQVSPEQKLISEEDLFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 69 - protein, 520 aa, Artificial Sequence
  synthetic construct
MEQGKGLAVLILAIILLQGTLAEQKLISEEDLQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIARSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 70 - protein, 627 aa, Artificial Sequence
  synthetic construct
MEHSTFLSGLVLATLLSQVSPEQKLISEEDLFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 71 - protein, 421 aa, Artificial Sequence
  synthetic construct
MEHSTFLSGLVLATLLSQVSPEQKLISEEDLFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKRAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 72 - protein, 321 aa, Artificial Sequence
  synthetic construct
MEHSTFLSGLVLATLLSQVSPEQKLISEEDLFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFARAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWS

SEQ ID NO: 73 - protein, 638 aa, Artificial Sequence
  synthetic construct
MEQGKGLAVLILAIILLQGTLAEQKLISEEDLQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 74 - protein, 432 aa, Artificial Sequence
  synthetic construct
MEQGKGLAVLILAIILLQGTLAEQKLISEEDLQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNRAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 75 - protein, 332 aa, Artificial Sequence
  synthetic construct
MEQGKGLAVLILAIILLQGTLAEQKLISEEDLQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIARAKRGSGEGRGSLLTCGDVEENPGPMQSGTHWRVLGLCLLSVGVWGQDYKDDDDKDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWS

SEQ ID NO: 76 - protein, 277 aa, Artificial Sequence
  synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQEQKLISEEDL

SEQ ID NO: 77 - skipped

SEQ ID NO: 78 - protein, 174 aa, Artificial Sequence
  synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREQKLISEEDL

SEQ ID NO: 79 - protein, 61 aa, Artificial Sequence
  synthetic construct
MKWKALFTAAILQAQLPITEAQSFGLLDPKLCYLLDGILFIYGVILTALFLEQKLISEEDL

SEQ ID NO: 80 - protein, 390 aa, Artificial Sequence
  synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQEQKLISEEDL

SEQ ID NO: 81 - protein, 287 aa, Artificial Sequence
  synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREQKLISEEDL

SEQ ID NO: 82 - protein, 390 aa, Artificial Sequence
  synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREQKLISEEDL

SEQ ID NO: 83 - protein, 277 aa, Artificial Sequence
  synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREQKLISEEDL

SEQ ID NO: 84 - protein, 273 aa, Artificial Sequence
  synthetic construct
MGHTRRQGTSPSKCPYLNFFQLLVLAGLSHFCSGEQKLISEEDLVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCL

SEQ ID NO: 85 - protein, 519 aa, Artificial Sequence
  synthetic construct
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPEQKLISEEDL

SEQ ID NO: 86 - protein, 519 aa, Artificial Sequence
  synthetic construct
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQFQPQPEQKLISEEDL

SEQ ID NO: 87 - protein, 781 aa, Artificial Sequence
  synthetic construct
MGHTRRQGTSPSKCPYLNFFQLLVLAGLSHFCSGEQKLISEEDLVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 88 - protein, 884 aa, Artificial Sequence
  synthetic construct
MGHTRRQGTSPSKCPYLNFFQLLVLAGLSHFCSGEQKLISEEDLVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 89 - protein, 781 aa, Artificial Sequence
  synthetic construct
MGHTRRQGTSPSKCPYLNFFQLLVLAGLSHFCSGEQKLISEEDLVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQFQPQP

SEQ ID NO: 90 - protein, 884 aa, Artificial Sequence
  synthetic construct
MGHTRRQGTSPSKCPYLNFFQLLVLAGLSHFCSGEQKLISEEDLVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQFQPQPRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 91 - protein, 293 aa, Artificial Sequence
  synthetic construct
MALPVTALLLPLALLLHAARPEQKLISEEDLMEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 92 - protein, 396 aa, Artificial Sequence
  synthetic construct
MALPVTALLLPLALLLHAARPEQKLISEEDLMEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 93 - protein, 468 aa, Artificial Sequence
  synthetic construct
MNRGVPFRHLLLVLQLALLPAATQGEQKLISEEDLKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 94 - protein, 571 aa, Artificial Sequence
  synthetic construct
MNRGVPFRHLLLVLQLALLPAATQGEQKLISEEDLKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 95 - protein, 488 aa, Artificial Sequence
  synthetic construct
MALPVTALLLPLALLLHAARPEQKLISEEDLSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRGSGEGRGSLLTCGDVEENPGPMRPRLWLLLAAQLTVLHGNSVDYKDDDDKLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 96 - protein, 694 aa, Artificial Sequence
  synthetic construct
MALPVTALLLPLALLLHAARPEQKLISEEDLSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQRAKRGSGEGRGSLLTCGDVEENPGPMRPRLWLLLAAQLTVLHGNSVDYKDDDDKLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 97 - protein, 221 aa, Artificial Sequence
  synthetic construct
MALPVTALLLPLALLLHAARPEQKLISEEDLELTDTLQAETDQLEDEKSALQTEIANLLKEKEKLEFILAAHNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 98 - protein, 202 aa, Artificial Sequence
  synthetic construct
MALPVTALLLPLALLLHAARPEQKLISEEDLELTDTLQAETDQLEDEKSALQTEIANLLKEKEKLEFILAAHFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 99 - protein, 209 aa, Artificial Sequence
  synthetic construct
MALPVTALLLPLALLLHAARPEQKLISEEDLLEEKVKTLKAQNSELASTANMLREQVAQLNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 100 - protein, 190 aa, Artificial Sequence
  synthetic construct
MALPVTALLLPLALLLHAARPEQKLISEEDLLEEKVKTLKAQNSELASTANMLREQVAQLFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 101 - protein, 209 aa, Artificial Sequence
  synthetic construct
MALPVTALLLPLALLLHAARPEQKLISEEDLETQHKVLELTAENERLQKKVEQLSRELSTNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 102 - protein, 190 aa, Artificial Sequence
  synthetic construct
MALPVTALLLPLALLLHAARPEQKLISEEDLETQHKVLELTAENERLQKKVEQLSRELSTFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 103 - protein, 781 aa, Artificial Sequence
  synthetic construct
MRPSGTAGAALLALLAALCPASRAEQKLISEEDLLEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 104 - protein, 324 aa, Artificial Sequence
  synthetic construct
MRPSGTAGAALLALLAALCPASRAEQKLISEEDLGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 105 - protein, 183 aa, Artificial Sequence
  synthetic construct
MRPSGTAGAALLALLAALCPASRAEQKLISEEDLNCTYGCTGPGLEGCPTNGPKIPSIATGMVGALLLLLVVALGIGLFMRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 106 - protein, 788 aa, Artificial Sequence
  synthetic construct
MELAALCRWGLLLALLPPGAASEQKLISEEDLTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 107 - protein, 788 aa, Artificial Sequence
  synthetic construct
MELAALCRWGLLLALLPPGAASEQKLISEEDLTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVEGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 108 - protein, 788 aa, Artificial Sequence
  synthetic construct
MELAALCRWGLLLALLPPGAASEQKLISEEDLTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVDILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 109 - protein, 788 aa, Artificial Sequence
  synthetic construct
MELAALCRWGLLLALLPPGAASEQKLISEEDLTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVRILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 110 - protein, 298 aa, Artificial Sequence
  synthetic construct
MELAALCRWGLLLALLPPGAASEQKLISEEDLQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 111 - protein, 298 aa, Artificial Sequence
  synthetic construct
MELAALCRWGLLLALLPPGAASEQKLISEEDLQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVEGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 112 - protein, 298 aa, Artificial Sequence
  synthetic construct
MELAALCRWGLLLALLPPGAASEQKLISEEDLQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVDILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 113 - protein, 298 aa, Artificial Sequence
  synthetic construct
MELAALCRWGLLLALLPPGAASEQKLISEEDLQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVRILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 114 - protein, 158 aa, Artificial Sequence
  synthetic construct
MELAALCRWGLLLALLPPGAASEQKLISEEDLSIISAVVGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 115 - protein, 158 aa, Artificial Sequence
  synthetic construct
MELAALCRWGLLLALLPPGAASEQKLISEEDLSIISAVEGILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 116 - protein, 158 aa, Artificial Sequence
  synthetic construct
MELAALCRWGLLLALLPPGAASEQKLISEEDLSIISAVVDILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 117 - protein, 158 aa, Artificial Sequence
  synthetic construct
MELAALCRWGLLLALLPPGAASEQKLISEEDLSIISAVVRILLVVVLGVVFGILIRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 118 - protein, 546 aa, Artificial Sequence
  synthetic construct
MALPVTALLLPLALLLHAARPEQKLISEEDLQEQLVESGGRLVTPGTPLTLTCTASGFSLGSDFMSWVRQAPGKGLEWIGYIDPRSDIPYYASWAKGRFTISKTSTTVDLKITSPTTEDTATYFCARDLNAGYFNGIFYIWGPGTLVTVSSGGGGSGGGGSGGGGSELVMTQTPSSVSAAVGDTVTINCQASETVATLLAWYQQKPGQPPKLLIYGASNLESGVPSRFRGSGSGTEFTLTISGMKAEDAATYYCQYGYISTGSNTFGAGTNVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 119 - protein, 546 aa, Artificial Sequence
  synthetic construct
MALPVTALLLPLALLLHAARPEQKLISEEDLELVMTQTPSSVSAAVGDTVTINCQASETVATLLAWYQQKPGQPPKLLIYGASNLESGVPSRFRGSGSGTEFTLTISGMKAEDAATYYCQYGYISTGSNTFGAGTNVEIKGGGGSGGGGSGGGGSQEQLVESGGRLVTPGTPLTLTCTASGFSLGSDFMSWVRQAPGKGLEWIGYIDPRSDIPYYASWAKGRFTISKTSTTVDLKITSPTTEDTATYFCARDLNAGYFNGIFYIWGPGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 120 - protein, 548 aa, Artificial Sequence
  synthetic construct
MALPVTALLLPLALLLHAARPEQKLISEEDLQEQLVESGGRLVTPGTPLTLTCTASGFSLGSDFMSWVRQAPGKGLEWIGYIDPRSDIPYYASWAKGRFTISKTSTTVDLKITSPTTEDTATYFCARDLNAGYFNGIFYIWGPGTLVTVSSGGGGSGGGGSGGGGSELDMTQTPSSTSEPVGGTVTINCQASQTISSYLSWYQQKPGHPPKLLIYDASDLASGVPSRFSGSRSGTQFTLTISGVQCDDAATYYCLGVYDYRSDDGAAFGGGTELEILAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 121 - protein, 548 aa, Artificial Sequence
  synthetic construct
MALPVTALLLPLALLLHAARPEQKLISEEDLELDMTQTPSSTSEPVGGTVTINCQASQTISSYLSWYQQKPGHPPKLLIYDASDLASGVPSRFSGSRSGTQFTLTISGVQCDDAATYYCLGVYDYRSDDGAAFGGGTELEILGGGGSGGGGSGGGGSQEQLVESGGRLVTPGTPLTLTCTASGFSLGSDFMSWVRQAPGKGLEWIGYIDPRSDIPYYASWAKGRFTISKTSTTVDLKITSPTTEDTATYFCARDLNAGYFNGIFYIWGPGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 122 - protein, 543 aa, Artificial Sequence
  synthetic construct
MALPVTALLLPLALLLHAARPEQKLISEEDLQSLEESGGRLVTPGTPLTLTCTVSGFSLSTNDMNWVRQAPGKGLEWIGVIYSDDTPDYATWAKGRFTISRTSTTVDLKITSPTTEDTATYFCARGHYDSAVYAYALNIWGPGTLVTVSSGGGGSGGGGSGGGGSELVMTQTPSSVSAAVGGTVTITCQASQSLSNLLAWYQQKPGQPPKLLIYGASNLESGVPSRFRGSGSGTDFTLTISGMKAEDAATYYCQGGHYSGLTFGNGTNVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 123 - protein, 543 aa, Artificial Sequence
  synthetic construct
MALPVTALLLPLALLLHAARPEQKLISEEDLELVMTQTPSSVSAAVGGTVTITCQASQSLSNLLAWYQQKPGQPPKLLIYGASNLESGVPSRFRGSGSGTDFTLTISGMKAEDAATYYCQGGHYSGLTFGNGTNVEIKGGGGSGGGGSGGGGSQSLEESGGRLVTPGTPLTLTCTVSGFSLSTNDMNWVRQAPGKGLEWIGVIYSDDTPDYATWAKGRFTISRTSTTVDLKITSPTTEDTATYFCARGHYDSAVYAYALNIWGPGTLVTVSSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 124 - protein, 226 aa, Artificial Sequence
  synthetic construct
MYGKIIFVLLLSEIVSISAEQKLISEEDLSSTTGVAMHTSTSSSVTKSYISSQTNDTHKRDTYAATPRAHEVSEISVRTVYPPEEETGERVQLAHHFSEPEITLIIFGVMAGVIGTILLISYGRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 125 - protein, 154 aa, Artificial Sequence
  synthetic construct
MYGKIIFVLLLSEIVSISAEQKLISEEDLITLIIFGVMAGVIGTILLISYGRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 126 - protein, 386 aa, Artificial Sequence
  synthetic construct
MDHLGASLWPQVGSLCLLLAGAAWEQKLISEEDLAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 127 - protein, 160 aa, Artificial Sequence
  synthetic construct
MDHLGASLWPQVGSLCLLLAGAAWEQKLISEEDLLILTLSLILVVILVLLTVLALLSRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 128 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 129 - protein, 162 aa, Artificial Sequence
  synthetic construct
MPSWALFMVTSCLLLAPQNLAQVSSEQKLISEEDLISLVTALHLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 130 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLNAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 131 - protein, 162 aa, Artificial Sequence
  synthetic construct
MPSWALFMVTSCLLLAPQNLAQVSSEQKLISEEDLISLVTALHLVLGLNAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 132 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 133 - protein, 162 aa, Artificial Sequence
  synthetic construct
MPSWALFMVTSCLLLAPQNLAQVSSEQKLISEEDLISLVTALHLVLGLSAVLGLLLLRKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 134 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALLLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 135 - protein, 162 aa, Artificial Sequence
  synthetic construct
MPSWALFMVTSCLLLAPQNLAQVSSEQKLISEEDLISLVTALLLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 136 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLNAVLGLLLLRKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 137 - protein, 162 aa, Artificial Sequence
  synthetic construct
MPSWALFMVTSCLLLAPQNLAQVSSEQKLISEEDLISLVTALHLVLGLNAVLGLLLLRKRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 138 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALYLVLGLNAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 139 - protein, 162 aa, Artificial Sequence
  synthetic construct
MPSWALFMVTSCLLLAPQNLAQVSSEQKLISEEDLISLVTALYLVLGLNAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 140 - protein, 628 aa, Artificial Sequence
  synthetic construct
MPSWALFMVTSCLLLAPQNLAQVSSEQKLISEEDLQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTAWCLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 141 - protein, 162 aa, Artificial Sequence
  synthetic construct
MPSWALFMVTSCLLLAPQNLAQVSSEQKLISEEDLISLVTAWCLVLGLSAVLGLLLLRWRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 142 - protein, 386 aa, Artificial Sequence
  synthetic construct
MALPVTALLLPLALLLHAARPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAAFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 143 - protein, 387 aa, Artificial Sequence
  synthetic construct
MALPVTALLLPLALLLHAARPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 144 - protein, 379 aa, Artificial Sequence
  synthetic construct
MALPVTALLLPLALLLHAARPERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 145 - protein, 430 aa, Artificial Sequence
  synthetic construct
MALPVTALLLPLALLLHAARPELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 146 - protein, 380 aa, Artificial Sequence
  synthetic construct
MALPVTALLLPLALLLHAARPESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 147 - protein, 380 aa, Artificial Sequence
  synthetic construct
MALPVTALLLPLALLLHAARPESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 148 - protein, 425 aa, Artificial Sequence
  synthetic construct
MALPVTALLLPLALLLHAARPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 149 - protein, 426 aa, Artificial Sequence
  synthetic construct
MALPVTALLLPLALLLHAARPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 150 - protein, 418 aa, Artificial Sequence
  synthetic construct
MALPVTALLLPLALLLHAARPERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 151 - protein, 469 aa, Artificial Sequence
  synthetic construct
MALPVTALLLPLALLLHAARPELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 152 - protein, 419 aa, Artificial Sequence
  synthetic construct
MALPVTALLLPLALLLHAARPESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 153 - protein, 419 aa, Artificial Sequence
  synthetic construct
MALPVTALLLPLALLLHAARPESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 154 - protein, 62 aa, Artificial Sequence
  synthetic construct
  MISC_FEATURE (1)..(61): CD40
KKVAKKPTNKAPHPKQEPQEINFPDDLPGSNTAAPVQETLHGCQPVTQEDGKESRISVQERQ

SEQ ID NO: 155 - protein, 124 aa, Artificial Sequence
  synthetic construct
  MISC_FEATURE (1)..(124): CD40_tandem
KKVAKKPTNKAPHPKQEPQEINFPDDLPGSNTAAPVQETLHGCQPVTQEDGKESRISVQERQKKVAKKPTNKAPHPKQEPQEINFPDDLPGSNTAAPVQETLHGCQPVTQEDGKESRISVQERQ

SEQ ID NO: 156 - protein, 62 aa, Artificial Sequence
  synthetic construct
  MISC_FEATURE (1)..(62): CD40_P227A
KKVAKKPTNKAAHPKQEPQEINFPDDLPGSNTAAPVQETLHGCQPVTQEDGKESRISVQERQ

SEQ ID NO: 157 - protein, 9 aa, Artificial Sequence
  synthetic construct
AAAGSGGSG

SEQ ID NO: 158 - protein, 15 aa, Artificial Sequence
  synthetic construct
GGGGSGGGGSGGGGS